(19) United States
(12) Patent Application Publication
(10) Pub. No.: US 2006/0057595 A1
(43) Pub. Date: Mar. 16, 2006

(54) Compositions, methods, and kits for identifying and quantitating small RNA molecules

(75) Inventors: Kai Qin Lao, Pleasanton, CA (US); Neil Straus, Emeryville, CA (US)

Correspondence Address: MILA KASAN, PATENT DEPT.;APPLIED BIOSYSTEMS, 850 LINCOLN CENTRE DRIVE, FOSTER CITY, CA, 94404, US

(73) Assignee: Applera Corporation, Foster City, CA (US)

(21) Appl. No.: 10/944,153

(22) Filed: Sep. 16, 2004

(51) Int. Cl. C12Q 1/68; C07H 21/02; C12P 19/34

(52) U.S. Cl. 435/6; 435/91.2; 536/23.1

(57) ABSTRACT

Compositions, methods, and kits for identifying and for quantitating polynucleotide targets are disclosed. These compositions, methods, and kits are particularly useful when the polynucleotide target is a small RNA molecule, including without limitation microRNA (miRNA), small interfering RNA (siRNA), and certain other classes of non-coding RNA molecules. The forward and reverse primers of the disclosed first primer sets comprise unusually short target-binding portions that are 6-10 nucleotides long. Certain of the disclosed methods employ one or more multiplex reaction steps to identify, quantitate, or identify and quantitate, a multiplicity of target polynucleotides.

### FIELD

The present teachings generally relate to methods, reagents, and kits for discovering, detecting, or quantifying small RNA molecules. More specifically, the disclosed compositions, methods, and kits are useful in identifying, detecting, and quantitating polynucleotides, including without limitation, miRNA precursors, polynucleotides comprising a deoxyribonucleotide, and small RNA molecules, for example but not limited to, microRNA (miRNA), small interfering RNA (siRNA), and other noncoding RNA (ncRNA) molecules.

### BACKGROUND

The identification and quantitation of specific nucleic acid sequences has been an area of great interest in molecular biology over the past two to three decades. Genotyping and gene expression profiling are but two areas that are currently being intensively studied. The ability to identify and to quantitate certain nucleic acids and their products has allowed the advancement of a broad range of disciplines, such as individualized medicine, including analyses of single nucleotide polymorphisms (SNPs) and evaluation of drug resistance, furthered our understanding of biochemical and molecular biological processes, and advanced cancer diagnosis and treatment, among others.

Recently much interest has focused on the newly discovered properties of certain non-coding small RNA molecules, particularly small interfering RNA (siRNA) and micro RNA (miRNA) and its precursors and their effect on intracellular processes. It is currently believed that siRNA is involved in gene silencing, while miRNA is believed to be responsible for some forms of translational repression and in certain instances, gene silencing. While the interest in these small RNA molecules has risen dramatically, scientists are faced with the difficult task of identifying and quantitating these small molecules.

The siRNA molecules are typically 19-23 nucleotides in length after Dicer cleavage. The miRNA are endogenous expressed single-stranded ribonucleotides that range in size from about 17 to about 29 nucleotides in length. miRNA are derived from specific genes that may or may not have their own regulatory sequences. miRNA species have been identified using molecular cloning techniques, computational algorithms such as MiRscan and miRseeker, and trial and error approaches. Several hundred miRNA species have been identified in C. elegans, Drosophila, plants, and mammals, including humans, and the number is increasing as additional miRNA species are discovered.

According to the currently accepted miRNA biogenesis model, the miRNA genes are transcribed to generate primary transcripts (pri-miRNA) that sometimes exceed 1 kilobase. The pri-miRNA is cleaved in the nucleus by the endonuclease Drosha to form precursor miRNAs (pre-miRNAs) that are typically about 70-80 nucleotides long. The pre-miRNAs are actively transported from the nucleus to the cytoplasm where they are further processed into miRNA by the endonuclease Dicer, which also participates in siRNA processing.

While much has been learned about various small RNA molecules in the past decade, much remains to be elucidated. Their small size can present problems, particularly with respect to identifying and validating candidate small RNA molecules, and detecting and quantifying known species of small RNA molecules. Conventional techniques do not adequately address these needs.

### SUMMARY

The present teachings are directed to methods, reagents, and kits for identifying, detecting, and quantitating polynucleotides, for example but not limited to, polynucleotides composed of deoxyribonucleotides and polynucleotides composed of ribonucleotides, including without limitation, small RNA molecules, such as untranslated functional RNA, non-coding RNA (ncRNA), small non-messenger RNA (snmRNA), siRNA, tRNA, tiny non-coding RNA (tncRNA), small modulatory RNA (smRNA), snoRNA, stRNA, snRNA, miRNA including without limitation miRNA precursors such as primary miRNA (pri-miRNA) and precursor miRNA (pre-miRNA), and so forth (see, e.g., Eddy, Nature Reviews Genetics 2:919-29, 2001; Storz, Science 296:1260-63, 2002; Buckingham, Horizon Symposia: Understanding the RNAissance:1-3, 2003).

First primer sets are disclosed that include a forward primer and a corresponding reverse primer, each with an unconventionally short target-binding portion. The target-binding portion of the forward primers comprise no more than ten nucleotides that have the same sequence as a first region of the corresponding polynucleotide target. The target-binding portion of the reverse primers comprise no more than ten nucleotides that are complementary to a second region of the corresponding polynucleotide target. In some embodiments, the target-binding portion of the forward and reverse primers of such first primer sets contain only six, seven, eight, or nine nucleotides that have the same sequence as, or are complementary to, the first and second regions of the corresponding polynucleotide target, respectively. In some embodiments, the corresponding region of the target comprises the terminal nucleotide on the 5′-end or the 3′-end of the target polynucleotide, while in other embodiments, the corresponding region of the target polynucleotide does not include the terminal 5′ nucleotide or the terminal 3′ nucleotide of the target polynucleotide. The forward and reverse primers of a first primer set typically further comprise a second portion that is upstream of the target-binding portion of the primer and which can, but need not, be a primer-binding portion. Primers comprising nucleotide analogs, within or outside of the target-binding portion, are also within the scope of the current teachings provided that such analogs do not interfere with the disclosed amplification steps.

In certain embodiments of the disclosed methods, a single reaction composition is formed comprising a polynucleotide target, a first primer set, and a first extending enzyme. In certain embodiments, the single reaction composition further comprises a second primer set, a second extending enzyme, a third extending enzyme, or combinations thereof. In essence, two primer sets per polynucleotide target are used in two, three, or four amplification steps that occur in the same reaction composition and typically, the same reaction vessel. The amplification steps typically include: (i) generating a first product by extending the reverse primer of the first primer set, (ii) generating a first amplicon using the first product as the template and the corresponding forward primer of the first primer set, (iii) optionally, generating additional first amplicons using additional forward and reverse primers of the corresponding first primer set, and (iv) optionally, generating second amplicons using the first amplicons (and where appropriate, also the additional first amplicons) as templates and the corresponding first and second primers of the second primer set, which can, but need not include universal primers, primers comprising unique hybridization tags, or both. The reaction can, but need not, comprise real-time detection. In certain embodiments, an amplification step comprises multiplexing.

In certain embodiments, a polynucleotide target is combined with a first primer set, comprising a forward primer and a reverse primer, a second primer set, and an extending enzyme to form a single reaction composition. The single reaction composition is reacted under appropriate conditions and a first product, a first amplicon, an additional first amplicon, a second amplicon are generated. In certain embodiments, a first amplicon, an additional first amplicon, a second amplicon, or combinations thereof, are detected and the polynucleotide is identified and/or quantitated. In certain embodiments, the detecting comprises an integral reporter group, a reporter probe, an intercalating agent, or combinations thereof. In certain embodiments, the amplifying, the detecting, and the quantitating comprise Q-PCR or another real time technique. Certain embodiments comprise an end-point detection technique.

In certain embodiments, the disclosed methods comprise forming at least two different reaction compositions, for example but not limited to, a first reaction composition and a second reaction composition. Some embodiments further comprise at least a third reaction composition. In certain embodiments, two primer sets per polynucleotide target are used in three or four amplification steps that occur in at least two different reaction compositions, including without limitation, a first reaction composition and a multiplicity of different second reaction compositions, and can but need not take place in the same reaction vessel. According to such methods, the amplification steps that occur in the first reaction compositions typically include: (i) generating a first product using the reverse primer of the first primer set, (ii) generating a first amplicon using the first product as the template and the corresponding forward primer of the first primer set, and optionally, (iii) generating additional first amplicons using additional forward and reverse primers of the corresponding first primer set. When the first stage is completed, a second reaction composition is typically formed by combining (i) all or part of the reacted first reaction composition, (ii) a second primer set, which can, but need not include universal primers, primers comprising unique hybridization tags, or both, (iii) a third extending enzyme, and optionally, (iv) a reporter probe. Under appropriate reaction conditions second amplicons are generated using the additional first amplicons as templates. In certain embodiments, the first stage reactions are performed in a multiplex first reaction composition. In certain embodiments, the second stage reaction is performed in multiplex, which can, but need not, include a multiplicity of parallel lower-plexy second reaction compositions. The second stage reaction can, but need not, include real-time detection.

Some of the disclosed methods comprise hybridizing a reverse primer of a first primer set with the second region of a corresponding polynucleotide target and extending the hybridized reverse primer using a first extending enzyme to generate a first product. When the target comprises RNA, for example but not limited to a small RNA molecule, the first product comprises a reverse-transcribed product and the first extending enzyme can, but need not be, a reverse transcriptase. The first product hybridizes with the corresponding forward primer from the first primer set and the hybridized forward primer is extended by a second extending enzyme to generate a first amplicon. The denatured strands of the first amplicon can hybridize with additional forward and reverse primers that can be extended to generate additional first amplicons. In certain embodiments, one strand of and/or double-stranded forms of a first amplicon, an additional first amplicon, or a first amplicon and an additional first amplicon is detected due to a reporter group in the first amplicon or the additional first amplicon, reporter probe binding, dye intercalation, or combinations thereof.

In certain embodiments, second amplicons are generated by hybridizing the primers of a second primer set with the corresponding strands of the additional first amplicons and extending the hybridized second primers with a third extending enzyme. In certain embodiments, the second extending enzyme and the third extending enzyme are the same enzyme, while in other embodiments, they are different enzymes. The amplification reaction comprising the second primer set can be cycled using the additional first amplicons, the second amplicons, or the additional first amplicons and the second amplicons, as templates in additional reaction cycles, to generate more second amplicons. The second amplicons, whether generated by a single amplification cycle or multiple amplification cycles, are detected and the corresponding polynucleotide target can be identified, quantitated, or identified and quantitated. In certain embodiments, a single- and/or double-stranded form of a second amplicon or its surrogate is detected due to a reporter group in the second amplicon, reporter probe binding, dye intercalation, or combinations thereof.

In certain embodiments, a second reaction composition is formed, comprising (i) the first amplicons, the additional first amplicons, or the first amplicons and the additional first amplicons, and (ii) a second primer set that comprises primers that can hybridize with the primer-binding portions of the first amplicons, the additional first amplicons, or the first amplicons and the additional first amplicons. In certain embodiments, the second reaction composition further comprises a reporter probe, an intercalating agent, a third extending enzyme (that may be the same or different from the a second extending enzyme), a reporter group-labeled dNTP, a dNTP comprising a linker arm, or combinations thereof. In certain embodiments of the disclosed methods, the target comprises a multiplicity of different target polynucleotides, including without limitation a multiplicity of polynucleotides comprising deoxyribonucleotides or polynucleotides comprising ribonucleotides, for example but not limited to a multiplicity of small RNA molecules. In certain embodiments, the second reaction composition comprises a multiplicity of second reaction compositions, wherein a subset of the multiplicity of polynucleotide targets are identified, and/or quantified.

Other embodiments of the disclosed methods for identifying a polynucleotide target or for quantitating a polynucleotide target comprise, forming a reaction composition comprising the polynucleotide target, a first primer set, a second primer set, and a first extending enzyme. In certain embodiments, the reaction composition further comprises a reporter probe, an intercalating agent, a reporter group-labeled dNTP, a dNTP comprising a linker arm, a second extending enzyme, a third extending enzyme, or combinations thereof. Under appropriate reaction conditions, a first product, a first amplicon, an additional first amplicon, and a second amplicon are generated. The second amplicons are detected and the corresponding polynucleotide target is identified, quantified, or identified and quantified. In certain embodiments, further amplification cycles generate additional first amplicons, more second amplicons, or both.

In certain embodiments of the disclosed methods, a multiplicity of different polynucleotide targets are identified, quantified, or identified and quantified, using a multiplicity of different first primer sets, a first extending enzyme, and a second extending enzyme, wherein the first extending enzyme and the second extending enzyme are the same enzyme or different enzymes. Some embodiments further comprise, a multiplicity of different second primer sets, wherein a second primer set can comprise a universal primer, a unique hybridization tag, or both; a third extending enzyme; a multiplicity of different reporter probes; a reporter group-labeled dNTP; a dNTP comprising a linker arm; or combinations thereof. In some embodiments, a reporter group-labeled dNTP, a dNTP comprising a linker arm, or both, are incorporated into an Amplicon. In certain embodiments, an affinity tag or a reporter group are bound to an Amplicon comprising a linker arm. In some embodiments, the second extending enzyme and the third extending enzyme are the same enzyme and the first extending enzyme and the second extending enzyme are different enzymes.

Certain embodiments of the current teachings include multiplex steps for identifying, detecting, or quantitating a multiplicity of different polynucleotides, including without limitation, small RNA molecules. Some embodiments comprise single-plex steps for identifying, detecting, or quantitating a single target polynucleotide. Certain embodiments of the current teachings comprise two or more multiplex steps. Certain embodiments contemplate multiplex methods comprising a single-plex reaction, a two-plex reaction, a three-plex reaction, a four-plex reaction, and so forth. In certain embodiments, only a subset of the multiplicity of different polynucleotide targets being evaluated are identified, detected, and/or quantitated in a given single-plex reaction, two-plex reaction, three-plex reaction, four-plex reaction, and so forth. Certain embodiments of the current teachings further comprise a multi-well reaction vessel, including without limitation, a multi-well plate or a multi-chambered microfluidic device, wherein a multiplicity of such subset analyses are performed, typically in parallel.

According to the present teachings, methods for identifying small RNA molecules are disclosed. In certain embodiments of such methods, a reverse primer of a first primer set is hybridized to the small RNA molecule. The reverse primer comprises: (a) a primer-binding portion that is upstream from (b) a RNA molecule-binding portion comprising no more than ten nucleotides that are complementary to a second region of the small RNA molecule (typically at or near the 3′-end). In certain embodiments, the small RNA molecule-binding portion of the reverse primer comprises six, seven, eight, or nine nucleotides that are complementary to the second region of the small RNA molecule. The hybridized reverse primer is extended along the small RNA molecule in a template-dependent manner by a first extending enzyme to generate a reverse-transcribed product. A forward primer of the corresponding first primer set, comprising: (a) a primer-binding portion that is upstream from (b) a small RNA molecule-binding portion comprising no more than ten nucleotides having the same sequence as a first region of the small RNA molecule (typically at or near the 5′-end), is hybridized to the corresponding reverse-transcribed product. The hybridized forward primer is extended by a second extending enzyme to generate a first amplicon. In certain embodiments, a first amplicon is denatured, the separated strands hybridize with either the forward primer or the reverse primer of the first primer set, as appropriate. The hybridized forward and reverse primers are extended by a second extending enzyme to generate an additional first amplicon. The cycle of (a) denaturing the first amplicon and/or additional first amplicon, (b) hybridizing the corresponding forward and reverse primers to the denatured strands of the first amplicon and/or the additional first amplicon, and (c) extending the hybridized primers using an extension enzyme can, but need not be, repeated to generate more additional first amplicons. Those in the art will understand that the first and the second target regions can, but need not, include the terminal nucleotide of the target polynucleotide; in some embodiments, they can stop at the penultimate nucleotide, the third nucleotide from the corresponding end, and so forth.

In certain embodiments, an additional first amplicon is combined with a second primer set and the first amplicon is amplified by a second extending enzyme or a third extending enzyme to generate a multiplicity of second amplicons. In certain embodiments, the first amplicon, the additional first amplicon, or the first amplicon and the additional first amplicon is combined with the second primer set and amplified by a second extending enzyme or a third extending enzyme to generate a multiplicity of second amplicons. The second amplicons can be detected by any of a variety of detection means and the small RNA molecule is identified and/or quantitated. In certain embodiments, the first extending enzyme and the second extending enzyme are the same enzyme or different enzymes. In certain embodiments, the second extending enzyme and the third extending enzyme are the same enzyme or different enzymes. In certain embodiments, the second extending enzyme and the third extending enzyme are the same enzyme and the first extending enzyme is a different enzyme. In certain embodiments, the detecting comprises a reporter probe, an intercalating agent, or both. In certain embodiments, the amplifying, the detecting, and the quantitating comprise quantitative PCR (Q-PCR) or another real time technique. Certain embodiments comprise an end-point detection technique.

Certain embodiments of the disclosed methods for identifying or for quantitating a target polynucleotide comprise: a step for generating a first product; a step for generating a first amplicon; a step for generating an additional first amplicon; a step for generating a second amplicon; a step for detecting the second amplicon or its surrogate; and a step for quantitating the polynucleotide target or a step for identifying the polynucleotide target. In certain embodiments, the polynucleotide target comprises a small RNA molecule, including without limitation, a miRNA.

The current teachings also provide reporter probes that are particularly useful in the disclosed methods. Those in the art will appreciate, however, that conventional reporter probes may also be used in the disclosed methods. Also provided are kits that can be used to perform the disclosed methods. In certain embodiments, kits comprise a first primer set and a first extending enzyme. In certain embodiments, the disclosed kits further comprise, a second extending enzyme, a third extending enzyme, a second primer set, a reporter probe, a reporter group, a reaction vessel, or combinations thereof. These and other features of the present teachings are set forth herein.

### BRIEF DESCRIPTION OF THE DRAWINGS

The skilled artisan will understand that the drawings, described below, are for illustration purposes only and are not intended to limit the scope of the present teachings in any way.

FIGS. 1A and 1B: provide a schematic overview of various aspects of certain embodiments of the current teachings. “F” represents a fluorescent reporter group and “Q” represents a quencher that form a fluorescent reporter group-quencher pair on an exemplary reporter probe.

FIG. 2: depicts a first primer (1; SEQ ID NO:215) of an illustrative first primer set that includes a target-binding portion (2) and, optionally, a second portion (3; a first primer-binding portion in this example) that is upstream from the target-binding portion (2); a polynucleotide target (4; SEQ ID NO:216) that includes a first target region (5) a second target region (6), and in this example, a stretch of gap sequences (7; shown underlined); and a corresponding reverse primer (8; SEQ ID NO:217) of the illustrative first primer set that includes a target-binding portion (9) and, optionally, a second portion (10; a second primer-binding portion in this example) that is upstream from target-binding portion (9).

FIG. 3: depicts aspects of certain embodiments of the current teachings that comprise a single reaction composition. Polynucleotide targets, first primer sets, extending enzymes, and a second primer set, are combined to form a first reaction composition. In certain embodiments, the first reaction composition further comprises corresponding reporter probes, including without limitation, when real-time instruments are employed. The first reaction composition is subjected to multiple cycles of denaturation, primer annealing, and extension, to generate second amplicons. In certain embodiments (shown below the uppermost left arrow), wherein the first reaction composition did not include reporter probes, the second amplicons are combined with reporter probes and, optionally, an extending enzyme (shown in parentheses). The reporter probes or their surrogates are detected and the corresponding target is identified and/or quantified. In the alternate embodiment depicted (right branch), the reporter probes were included in the first reaction composition and detection, identification and/or quantitation of the corresponding polynucleotide occurs during cycling, for example but not limited to, using real-time analysis techniques.

FIG. 4: depicts certain aspects of various embodiments of certain multiplex methods that employ a two stage, two reaction composition format. A multiplicity of polynucleotide targets, A-F for illustration purposes, is combined with corresponding first primer sets and an extending enzyme to form a first reaction composition. First amplicons and additional first amplicons are generated when the first reaction composition is subjected to a limited number of cycles of denaturation, forward and/or reverse primer annealing, and extension. The reacted first reaction composition is diluted in an appropriate diluent and divided into three aliquots, which are distributed into three different second reaction compositions. Each of the three exemplary second reaction compositions comprise an aliquot of the diluted reacted first reaction composition, appropriate second primer sets (depicted as SPS A/B, SPS C/D, and SPS E/F), two different reporter probes per second reaction composition (either A and B, C and D, or E and F, depicted as RPA, RPB, RPC, RPD, RPE, and RPF, respectively), and an extending enzyme (EE). These second reaction compositions are subjected to multiple cycles of denaturation, primer annealing, and extension and corresponding second amplicons are generated (depicted as SAA, SAB, SAC, SAD, SAE, and SAF). In this illustrative embodiment, real-time analysis is employed to detect, identify and/or quantitate the target polynucleotides while the different second reaction compositions are cycled.

### DESCRIPTION OF EXEMPLARY EMBODIMENTS

It is to be understood that both the foregoing general description and the following detailed description are exemplary and explanatory only and are not intended to limit the scope of the current teachings. In this application, the use of the singular includes the plural unless specifically stated otherwise. For example, “a primer” means that more than one primer can, but need not, be present; for example but without limitation, one or more copies of a particular first primer species, as well as one or more versions of a particular primer type, for example but not limited to, a multiplicity of different forward primers. Also, the use of “comprise”, “comprises”, “comprising”, “contain”, “contains”, “containing”, “include”, “includes”, and “including” are not intended to be limiting.

The section headings used herein are for organizational purposes only and are not to be construed as limiting the described subject matter in any way. All literature and similar materials cited in this application, including but not limited to, patents, patent applications, articles, books, treatises, and internet web pages are expressly incorporated by reference in their entirety for any purpose. In the event that one or more of the incorporated literature and similar materials contradicts this application, including but not limited to defined terms, term usage, described techniques, or the like, this application controls.

### I. Definitions

The term “affinity tag” as used herein refers to a component of a multi-component complex, wherein the components of the multi-component complex specifically interact with or bind to each other. Exemplary multiple-component affinity tag complexes include without limitation, ligands and their receptors, for example but not limited to, avidin-biotin, streptavidin-biotin, and derivatives of biotin, streptavidin or avidin, including without limitation, 2-iminobiotin, desthiobiotin, NeutrAvidin (Molecular Probes, Eugene, Oreg.), CaptAvidin (Molecular Probes), and the like; binding proteins/peptides and their binding partners, including without limitation, maltose-maltose binding protein (MBP), calcium-calcium binding protein/peptide (CBP); epitope tags, for example but not limited to c-MYC (e.g., EQKLISEEDL), HA (e.g., YPYDVPDYA), VSV-G (e.g., YTDIEMNRLGK), HSV (e.g., QPELAPEDPED), V5 (e.g., GKPIPNPLLGLDST), and FLAG Tag™ (e.g., DYKDDDDKG), and their corresponding anti-epitope antibodies; haptens, for example but not limited to dinitrophenol (“DNP”) and digoxigenin (“DIG”), and their corresponding antibodies; aptamers and their binding partners; poly-His tags (e.g., penta-His and hexa-His) and their binding partners, including without limitation, corresponding metal ion affinity chromatography (IMAC) materials and anti-poly-His antibodies; fluorophores and their corresponding anti-fluorophore antibodies; and the like. In certain embodiments, affinity tags are part of a separating means, part of a detecting means, or both.

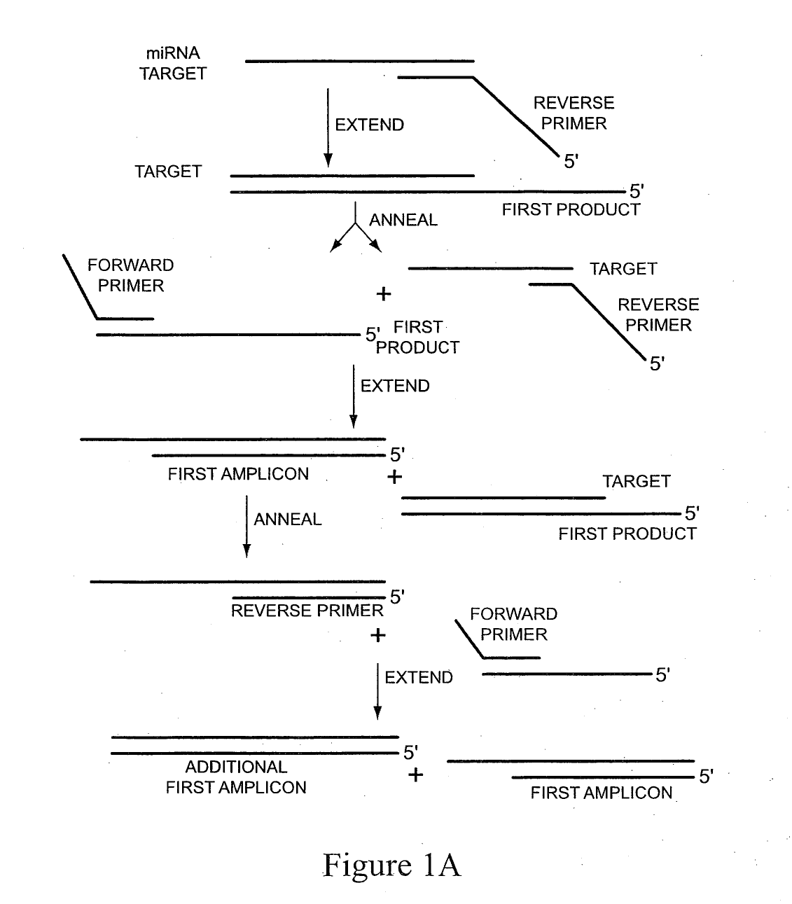

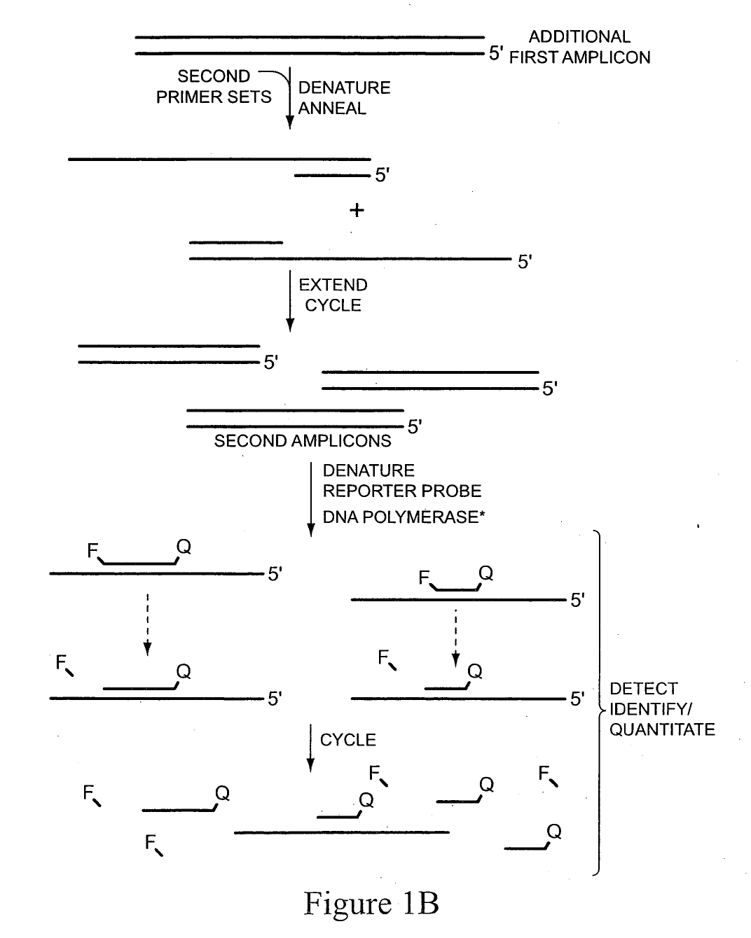

The term “Amplicons” is used in a broad sense herein and includes amplification products of the disclosed methods. First products (including but not limited to reverse transcribed products), first amplicons, additional first amplicons, second amplicons, or combinations thereof, fall within the intended scope of the term Amplicons (see, e.g., FIG. 1).

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AAB, BBC, AAABCCCC, CBBAAA, CABABB, and so forth. The skilled artisan will understand that typically there is no limit on the number of items or terms in any combination, unless otherwise apparent from the context.

The term “corresponding” as used herein refers to a specific relationship between the elements to which the term refers. For example, a forward primer of a particular first primer set corresponds to a reverse primer of the same first primer set, and vice versa. A second primer is designed to anneal with the primer-binding portion of a corresponding first product, a corresponding first amplicon, a corresponding second amplicon, or combinations thereof, depending on the context. The target-specific portion of a forward primer is designed to anneal with the complement of the first region of the corresponding polynucleotide target. The target-specific portion of a reverse primer is designed to anneal with the second region of the corresponding polynucleotide target. A particular affinity tag binds to the corresponding affinity tag, for example but not limited to, biotin binding to streptavidin. A particular hybridization tag anneals with its corresponding hybridization tag complement; and so forth.

The term “enzymatically active mutants or variants thereof” when used in reference to one or more enzyme, such as a DNA polymerase, including a reverse transcriptase, refers to one or more polypeptide derived from the corresponding enzyme that retains at least some of the desired enzymatic activity. Also within the scope of this term are: enzymatically active fragments, including but not limited to, cleavage products, for example but not limited to Klenow fragment, Stoffel fragment, or recombinantly expressed fragments and/or polypeptides that are smaller in size than the corresponding enzyme or that contains a sequence that is the same as part of, but not all of, the corresponding enzyme; mutant forms of the corresponding enzyme, including but not limited to, naturally-occurring mutants, such as those that vary from the “wild-type” or consensus amino acid sequence, mutants that are generated using physical and/or chemical mutagens, and genetically engineered mutants, for example but not limited to mutants generated using random and site-directed mutagenesis techniques; amino acid insertions and deletions, and changes due to nucleic acid nonsense mutations, missense mutations, and frameshift mutations; reversibly modified polymerases, for example but not limited to those described in U.S. Pat. No. 5,773,258; biologically active polypeptides obtained from gene shuffling techniques (see, e.g., U.S. Pat. Nos. 6,319,714 and 6,159,688), splice variants, both naturally occurring and genetically engineered, provided that they are derived, at least in part; from one or more corresponding enzymes; polypeptides corresponding at least in part to one or more such enzymes that comprise modifications to one or more amino acids of the native sequence, including without limitation, adding, removing or altering glycosylation, disulfide bonds, hydroxyl side chains, and phosphate side chains, or crosslinking, provided such modified polypeptides retain at least some of the desired catalytic activity; and the like. Expressly within the meaning of the term “enzymatically active mutants or variants thereof” when used in reference to a particular enzyme are enzymatically active mutants of that enzyme, enzymatically active variants of that enzyme, or enzymatically active mutants of that enzyme and enzymatically active variants of that enzyme.

The skilled artisan will readily be able to measure enzymatic activity using an appropriate assay known in the art. Thus, an appropriate assay for DNA polymerase catalytic activity might include, for example, measuring the ability of an enzyme variant to incorporate, under appropriate conditions, suitable deoxyribonucleotide triphosphates (dNTPs) into a nascent polynucleotide strand in a template-dependent manner. Protocols for such assays may be found in, among other places, Molecular Cloning, A Laboratory Manual, Cold Spring Harbor Press, 3d ed., 2001 (“Sambrook and Russell”); Sambrook, Fritsch, and Maniatis, Molecular Cloning, A Laboratory Manual, Cold Spring Harbor Press, 2d ed., 1989 (“Sambrook et al.”); Ausbel et al., Current Protocols in Molecular Biology, John Wiley & Sons, Inc. (including supplements through August 2004)(“Ausbel et al.”). It is to be understood that when a particular enzyme or type of enzyme (including without limitation, a first extending enzyme, a second extending enzyme, a third extending enzyme, a DNA polymerase, a reverse transcriptase, and so forth) is identified or claimed, the enzymatically active mutants or variants of that enzyme or type of enzyme are included, unless expressly stated otherwise.

The terms “groove binder” and “minor groove binder” are used interchangeably and refer to small molecules that fit into the minor groove of double-stranded DNA, typically in a sequence specific manner. Generally, minor groove binders are long, flat molecules that can adopt a crescent-like shape and thus, fit snugly into the minor groove of a double helix, often displacing water. Minor groove binding molecules typically comprise several aromatic rings connected by bonds with torsional freedom, such as but not limited to, furan, benzene, or pyrrole rings. Exemplary minor groove binders include without limitation, antibiotics such as netropsin, distamycin, berenil, pentamidine and other aromatic diamidines, Hoechst 33258, SN 6999, aureolic anti-tumor drugs such as chromomycin and mithramycin, CC-1065, dihydrocyclopyrroloindole tripeptide (DPI₃), 1,2-dihydro-(3H)-pyrrolo[3,2-e]indole-7-carboxylate (CDPI₃), and related compounds and analogues. In certain embodiments, a minor groove binder is a component of a primer, a reporter probe, a hybridization tag complement, or combinations thereof. Detailed descriptions of minor groove binders can be found in, among other places, Nucleic Acids in Chemistry and Biology, 2d ed., Blackburn and Gait, eds., Oxford University Press, 1996 (“Blackburn and Gait”), particularly in section 8.3; Kumar et al., Nucl. Acids Res. 26:831-38,1998; Kutyavin et al., Nucl. Acids Res. 28:655-61, 2000; Turner and Denny, Curr. Drug Targets 1:1-14, 2000; Kutyavin et al., Nucl. Acids Res. 25:3718-25, 1997; Lukhtanov et al., Bioconjug. Chem. 7:564-7, 1996; Lukhtanov et al., Bioconjug. Chem. 6: 418-26, 1995; U.S. Pat. No. 6,426,408; and PCT Published Application No. WO 03/078450. Those in the art understand that minor groove binders typically increase the Tₘ of the primer or the reporter probe to which they are attached, allowing such primers or reporter probes to effectively hybridize at higher temperatures. Primers and reporter probes comprising minor groove binders are commercially available from, among other sources, Applied Biosystems (Foster City, Calif.) and Epoch Biosciences (Bothell, Wash.).

The terms “hybridizing” and “annealing”, and variations of these terms such as annealed, hybridization, anneal, hybridizes, and so forth, are used interchangeably and mean the nucleotide base-pairing interaction of one nucleic acid with another nucleic acid that results in the formation of a duplex, triplex, or other higher-ordered structure. The primary interaction is typically nucleotide base specific, e.g., A:T, A:U and G:C, by Watson-Crick and Hoogsteen-type hydrogen bonding. In certain embodiments, base-stacking and hydrophobic interactions may also contribute to duplex stability. Conditions under which reporter probes and primers hybridize to complementary and substantially complementary target sequences are well known in the art, e.g., as described in Nucleic Acid Hybridization, A Practical Approach, B. Hames and S. Higgins, eds., IRL Press, Washington, D.C. (1985) and J. Wetmur and N. Davidson, Mol. Biol. 31:349 et seq. (1968). In general, whether such annealing takes place is influenced by, among other things, the length of the hybridizing region of the primers and reporter probes and their complementary sequences, the pH, the temperature, the presence of mono- and divalent cations, the proportion of G and C nucleotides in the hybridizing region, the viscosity of the medium, and the presence of denaturants. Such variables influence the time required for hybridization. The presence of certain nucleotide analogs or groove binders in the primer or reporter probe can also influence hybridization conditions. Thus, the preferred annealing conditions will depend upon the particular application. Such conditions, however, can be routinely determined by persons of ordinary skill in the art, without undue experimentation.

The term “hybridization tag” as used herein refers to an oligonucleotide sequence that can be used for: separating the element (e.g., first amplicons, additional first amplicons, second amplicons, surrogates of any of these, ZipChute™ reagents, etc.) of which it is a component or to which it is hybridized, including without limitation, bulk separation; tethering or attaching the element to which it is bound to a substrate, which may or may not include separating; annealing a corresponding hybridization tag complement; or combinations thereof. In certain embodiments, the same hybridization tag is used with a multiplicity of different elements to effect bulk separation, substrate attachment, or combinations thereof. In certain embodiments, a hybridization tag provides a unique “address” or identifier to the element containing the hybridization tag. In certain embodiments, this address can be used to identify the corresponding element, for example but not limited to, hybridizing to a particular address or position on an ordered array comprising a corresponding hybridization tag complement (sometimes referred to as a Zip Code and Zip Code complement). In certain embodiments, a primer comprising a unique hybridization tag is incorporated into an Amplicon so that the hybridization tag can be subsequently used to bind a reporter probe for detecting that Amplicon (see, e.g., U.S. Pat. No. 6,270,967). A “hybridization tag complement” typically refers to an oligonucleotide that comprises a nucleotide sequence that is complementary to at least part of the corresponding hybridization tag. In various embodiments, hybridization tag complements serve as capture moieties for attaching a hybridization tag:element complex to a substrate for identification, such as multiplex decoding on a microarray, or other purposes; serve as “pull-out” sequences for bulk separation procedures; or both as capture moieties and as pull-out sequences. In certain embodiments, a hybridization tag complement comprises a reporter group, a mobility modifier, a reporter probe-binding portion, or combinations thereof. In certain embodiments, a hybridization tag complement is annealed to a corresponding hybridization tag and, subsequently, at least part of that hybridization tag complement is released and detected. In certain embodiments, determining comprises detecting one or more reporter groups on or attached to a hybridization tag complement or at least part of a hybridization tag complement.

Typically, hybridization tags and their corresponding hybridization tag complements are selected to minimize: internal self-hybridization; and cross-hybridization with different hybridization tag species, nucleotide sequences in a reaction composition, including but not limited to target or background sequences, different species of hybridization tag complements, target-specific portions of primers, and the like; but should be amenable to facile hybridization between the hybridization tag and its corresponding hybridization tag complement. Hybridization tag sequences and hybridization tag complement sequences can be selected by any suitable method, for example but not limited to, computer algorithms such as described in PCT Publication Nos. WO 96/12014 and WO 96/41011 and in European Publication No. EP 799,897; and the algorithm and parameters of SantaLucia (Proc. Natl. Acad. Sci. 95:1460-65, 1998). Descriptions of hybridization tags can be found in, among other places, U.S. Pat. No. 6,309,829 (referred to as “tag segment” therein); U.S. Pat. No. 6,451,525 (referred to as “tag segment” therein); U.S. Pat. No. 6,309,829 (referred to as “tag segment” therein); U.S. Pat. No. 5,981,176 (referred to as “grid oligonucleotides” therein); U.S. Pat. No. 5,935,793 (referred to as “identifier tags” therein); and PCT Publication No. WO 01/92579 (referred to as “addressable support-specific sequences” therein); and Gerry et al., J. Mol. Biol. 292:251-262, 1999) (referred to as “zip-codes” and “zip-code complements” therein). Those in the art will appreciate that a hybridization tag and its corresponding hybridization tag complement are, by definition, complementary to each other and that the terms hybridization tag and hybridization tag complement are relative and can essentially be used interchangeably in most contexts.

Hybridization tags can be located at or near the end of a primer, an Amplicon, a reporter probe, or combinations thereof; or they can be located internally. In certain embodiments, a hybridization tag is attached to a primer, an Amplicon, a reporter probe, or combinations thereof, via a linker arm. In certain embodiments, the linker arm is cleavable.

In certain embodiments, hybridization tags are at least 12 bases in length, at least 15 bases in length, 12-60 bases in length, or 15-30 bases in length. In certain embodiments, a hybridization tag is 12, 15, 20, 21, 22, 23, 24, 25, 26, 27, 28, 29, 30, 45, or 60 bases in length. In certain embodiments, at least two hybridization tag:hybridization tag complement duplexes have melting temperatures that fall within a ΔTₘ range (Tₘₐₓ-Tₘᵢₙ) of no more than 10° C. of each other. In certain embodiments, at least two hybridization tag:hybridization tag complement duplexes have melting temperatures that fall within a ΔTₘ range of 5° C. or less of each other.

The term “mobility-dependent analytical technique” as used herein, refers to any means for separating different molecular species based on differential rates of migration of those different molecular species in one or more separation techniques. Exemplary mobility-dependent analytical techniques include electrophoresis, chromatography, mass spectrometry, sedimentation, e.g., gradient centrifugation, field-flow fractionation, multi-stage extraction techniques, and the like. Descriptions of mobility-dependent analytical techniques can be found in, among other places, U.S. Pat. Nos. 5,470,705, 5,514,543, 5,580,732, 5,624,800, and 5,807,682; PCT Publication No. WO 01/92579; D. R. Baker, Capillary Electrophoresis, Wiley-Interscience (1995); Biochromatography: Theory and Practice, M. A. Vijayalakshmi, ed., Taylor & Francis, London, U.K. (2003); Krylov and Dovichi, Anal. Chem. 72:111R-128R (2000); Swinney and Bornhop, Electrophoresis 21:1239-50 (2000); Crabtree et al., Electrophoresis 21:1329-35 (2000); and A. Pingoud et al., Biochemical Methods: A Concise Guide for Students and Researchers, Wiley-VCH Verlag GmbH, Weinheim, Germany (2002).

The term “mobility modifier” as used herein refers to a molecular entity, for example but not limited to, a polymer chain, that when added to an element (e.g., a reporter probe, a primer, an Amplicon, or combinations thereof) affects the mobility of the element to which it is hybridized or bound, covalently or non-covalently, in a mobility-dependent analytical technique.

Typically, a mobility modifier changes the charge/translational frictional drag when hybridized or bound to the element; or imparts a distinctive mobility, for example but not limited to, a distinctive elution characteristic in a chromatographic separation medium or a distinctive electrophoretic mobility in a sieving matrix or non-sieving matrix, when hybridized or bound to the corresponding element; or both (see, e.g., U.S. Pat. Nos. 5,470,705 and 5,514,543; Grossman et al., Nucl. Acids Res. 22:4527-34, 1994). In certain embodiments, a multiplicity of different Amplicons that do not comprise mobility modifiers have the same or substantially the same mobility in a mobility-dependent analytical technique. Typically, such Amplicons can be separated or substantially separated in a mobility-dependent analytical technique when each Amplicon species further comprises an appropriate mobility modifier.

As used herein, the terms “polynucleotide”, “oligonucleotide”, “nucleic acid”, and “nucleic acid sequence” are generally used interchangeably and include single-stranded and double-stranded polymers of nucleotide monomers, including 2′-deoxyribonucleotides (DNA) and ribonucleotides (RNA) linked by inter-nucleotide phosphodiester bond linkages, or inter-nucleotide analogs, and associated counter ions, e.g., H⁺, NH₄⁺, trialkylammonium, tetraalkylammonium, Mg²⁺, Na⁺, and the like. A nucleic acid may be composed entirely of deoxyribonucleotides, entirely of ribonucleotides, or chimeric mixtures thereof. The nucleotide monomer units may comprise any of the nucleotides described herein, including, but not limited to, naturally occurring nucleotides and nucleotide analogs. Nucleic acids typically range in size from a few monomeric units, e.g. 5-40, when they are sometimes referred to in the art as oligonucleotides, to several thousands of monomeric nucleotide units. Nucleic acid sequences are shown in the 5′ to 3′ orientation from left to right, unless otherwise apparent from the context or expressly indicated differently; and in such sequences, “A” denotes adenine, “C” denotes cytosine, “G” denotes guanine, “T” denotes thymine, and “U” denotes uracil, unless otherwise apparent from the context.

The term “nucleotide base”, as used herein, refers to a substituted or unsubstituted aromatic ring or rings. In certain embodiments, the aromatic ring or rings contain a nitrogen atom. In certain embodiments, the nucleotide base is capable of forming Watson-Crick or Hoogsteen-type hydrogen bonds with a complementary nucleotide base. Exemplary nucleotide bases and analogs thereof include, but are not limited to, naturally-occurring nucleotide bases adenine, guanine, cytosine, 5 methyl-cytosine, uracil, thymine, and analogs of the naturally occurring nucleotide bases, including without limitation, 7-deazaadenine, 7-deazaguanine, 7-deaza-8-azaguanine, 7-deaza-8-azaadenine, N6-Δ2-isopentenyladenine (6iA), N6-Δ2-isopentenyl-2-methylthioadenine (2ms6iA), N2-dimethylguanine (dmG), 7-methylguanine (7mG), inosine, nebularine, 2-aminopurine, 2-amino-6-chloropurine, 2,6-diaminopurine, hypoxanthine, pseudouridine, pseudocytosine, pseudoisocytosine, 5-propynylcytosine, isocytosine, isoguanine, 7-deazaguanine, 2-thiopyrimidine, 6-thioguanine, 4-thiothymine, 4-thiouracil, O⁶-methylguanine, N⁶-methyladenine, O⁴-methylthymine, 5,6-dihydrothymine, 5,6-dihydrouracil, pyrazolo[3,4-D]pyrimidines (see, e.g., U.S. Pat. Nos. 6,143,877 and 6,127,121 and PCT Published Application WO 01/38584), ethenoadenine, indoles such as nitroindole and 4-methylindole, and pyrroles such as nitropyrrole. Certain exemplary nucleotide bases can be found, e.g., in Fasman, 1989, Practical Handbook of Biochemistry and Molecular Biology, pp. 385-394, CRC Press, Boca Raton, Fla., and the references cited therein.

The term “nucleotide”, as used herein, refers to a compound comprising a nucleotide base linked to the C-1′ carbon of a sugar, such as ribose, arabinose, xylose, and pyranose, and sugar analogs thereof. The term nucleotide also encompasses nucleotide analogs. The sugar may be substituted or unsubstituted. Substituted ribose sugars include, but are not limited to, those riboses in which one or more of the carbon atoms, for example the 2′-carbon atom, is substituted with one or more of the same or different, , —R, —OR, —NR₂ azide, cyanide or halogen groups, where each R is independently H, C₁-C₆ alkyl, C₂-C₇ acyl, or C₅-C₁₄ aryl. Exemplary riboses include, but are not limited to, 2′-(C1-C6)alkoxyribose, 2′-(C5-C14)aryloxyribose, 2′,3′-didehydroribose, 2′-deoxy-3′-haloribose, 2′-deoxy-3′-fluororibose, 2′-deoxy-3′-chlororibose, 2′-deoxy-3′-aminoribose, 2′-deoxy-3′-(C1-C6)alkylribose, 2′-deoxy-3′-(C1-C6)alkoxyribose and 2′-deoxy-3′-(C5-C14)aryloxyribose, ribose, 2′-deoxyribose, 2′,3′-dideoxyribose, 2′-haloribose, 2′-fluororibose, 2′-chlororibose, and 2′-alkylribose, e.g., 2′-O-methyl, 4′-α-anomeric nucleotides, 1′-α-anomeric nucleotides, 2′-4′- and 3′-4′-linked and other “locked” or “LNA”, bicyclic sugar modifications (see, e.g., PCT Published Application Nos. WO 98/22489, WO 98/39352, and WO 99/14226; and Braasch and Corey, Chem. Biol. 8:1-7, 2001). “LNA” or “locked nucleic acid” is a DNA analogue that is conformationally locked such that the ribose ring is constrained by a methylene linkage between, for example but not limited to, the 2′-oxygen and the 3′- or 4′-carbon or a 3′-4′ LNA with a 2′-5′ backbone. The conformation restriction imposed by the linkage often increases binding affinity for complementary sequences and increases the thermal stability of such duplexes. Exemplary LNA sugar analogs within a polynucleotide include, but are not limited to, the structures: 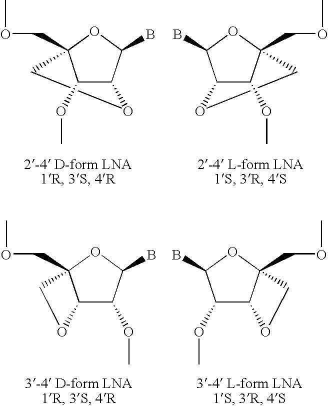
where B is any nucleotide base.

The 2′- or 3′-position of ribose can be modified to include, without limitation, hydrogen, hydroxy, methoxy, ethoxy, allyloxy, isopropoxy, butoxy, isobutoxy, methoxyethyl, alkoxy, phenoxy, azido, cyano, amido, imido, amino, alkylamino, fluoro, chloro and bromo. Nucleotides include, but are not limited to, the natural D optical isomer, as well as the L optical isomer forms (see, e.g., Garbesi Nucl. Acids Res. 21:4159-65 (1993); Fujimori (1990) J. Amer. Chem. Soc. 112:7435; Urata, (1993) Nucleic Acids Symposium Ser. No. 29:69-70). When the nucleotide base is purine, e.g., A or G, the ribose sugar is attached to the N⁹-position of the nucleotide base. When the nucleotide base is pyrimidine, e.g. C, T, or U, the pentose sugar is attached to the N¹-position of the nucleotide base, except for pseudouridines, in which the pentose sugar is attached to the C5 position of the uracil nucleotide base (see, e.g., Kornberg and Baker, (1992) DNA Replication, 2^{nd} Ed., Freeman, San Francisco, Calif.).

One or more of the pentose carbons of a nucleotide may be substituted with a phosphate ester having the formula: 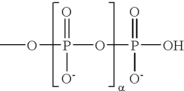
where α is an integer from 0 to 4. In certain embodiments, α is 2 and the phosphate ester is attached to the 3′- or 5′-carbon of the pentose. In certain embodiments, the nucleotides are those in which the nucleotide base is a purine, a 7-deazapurine, a pyrimidine, or an analog thereof. “Nucleotide 5′-triphosphate” refers to a nucleotide with a triphosphate ester group at the 5′ position, and is sometimes denoted as “NTP”, or “dNTP” and “ddNTP” to particularly point out the structural features of the ribose sugar. The triphosphate ester group may include sulfur substitutions for the various oxygens, e.g. α-thio-nucleotide 5′-triphosphates. Reviews of nucleotide chemistry can be found in, among other places, Shabarova, Z. and Bogdanov, A. Advanced Organic Chemistry of Nucleic Acids, VCH, New York, 1994; and Blackburn and Gait.

The term “nucleotide analog”, as used herein, refers to embodiments in which the pentose sugar or the nucleotide base or one or more of the phosphate esters of a nucleotide may be replaced with its respective analog. In certain embodiments, exemplary pentose sugar analogs are those described above. In certain embodiments, the nucleotide analogs have a nucleotide base analog as described above. In certain embodiments, exemplary phosphate ester analogs include, but are not limited to, alkylphosphonates, methylphosphonates, phosphoramidates, phosphotriesters, phosphorothioates, phosphorodithioates, phosphoroselenoates, phosphorodiselenoates, phosphoroanilothioates, phosphoroanilidates, phosphoroamidates, boronophosphates, etc., and may include associated counterions.

Also included within the definition of nucleotide analog are monomers that can be polymerized into polynucleotide analogs in which the DNA/RNA phosphate ester or sugar phosphate ester backbone is replaced at least in part by a different type of inter-nucleotide linkage. Exemplary polynucleotide analogs include, but are not limited to, peptide nucleic acids (PNAs), in which the sugar phosphate backbone of the polynucleotide is replaced by a peptide backbone comprising a amide bond. It is to be understood that the term “PNA” as used herein, includes pseudocomplementary PNAs (pcPNAs) unless otherwise apparent from the context. (See, e.g., Datar and Kim, Concepts in Applied Molecular Biology, Eaton Publishing, Westborough, Mass., 2003, particularly at pages 74-75; Verma and Eckstein, Ann. Rev. Biochem. 67:99-134, 1998; Goodchild, Bioconj. Chem., 1:165-187, 1990; Braasch and Corey, Methods 23:97-107, 2001; Demidov et al., Proc. Natl. Acad. Sci. 99:5953-58, 1999).

Nucleic acids include, but are not limited to, genomic DNA, cDNA, hnRNA, mRNA, rRNA, tRNA, small RNA molecules, including without limitation, miRNA and miRNA precursors, siRNA, stRNA, snoRNA, other non-coding RNAs (ncRNA), fragmented nucleic acid, nucleic acid obtained from the nucleus, the cytoplasm, subcellular organelles such as mitochondria or chloroplasts, and nucleic acid obtained from microorganisms or DNA or RNA viruses that may be present on or in a biological sample.

Nucleic acids may be composed of a single type of sugar moiety, e.g., as in the case of RNA and DNA, or mixtures of different sugar moieties, e.g., as in the case of RNA/DNA chimeras. In certain embodiments, nucleic acids are ribopolynucleotides and 2′-deoxyribopolynucleotides according to the structural formulae below: 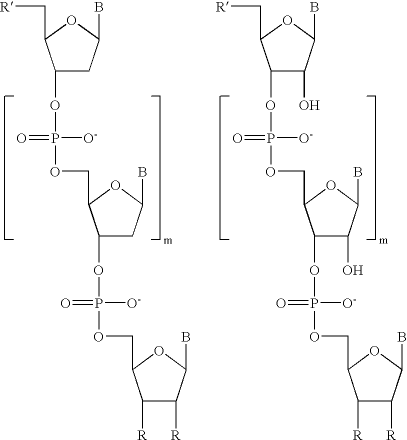
wherein each B is independently the base moiety of a nucleotide, e.g., a purine, a 7-deazapurine, a purine or purine analog substituted with one or more substituted hydrocarbons, a pyrimidine, a pyrimidine or pyrimidine analog substituted with one or more substituted hydrocarbons, or an analog nucleotide; each m defines the length of the respective nucleic acid and can range from zero to thousands, tens of thousands, or even more; each R is independently selected from the group comprising hydrogen, halogen, —R″, —OR″, and —NR″R″, where each R″ is independently (C1-C6) alkyl, (C2-C7) acyl or (C5-C14) aryl, cyanide, azide, or two adjacent Rs are taken together to form a bond such that the ribose sugar is 2′,3′-didehydroribose; and each R′ is independently hydroxyl or 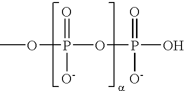
where α is zero, one or two.

In certain embodiments of the ribopolynucleotides and 2′-deoxyribopolynucleotides illustrated above, the nucleotide bases B are covalently attached to the C1′ carbon of the sugar moiety as previously described. The terms “nucleic acid”, “nucleic acid sequence”, “polynucleotide”, and “oligonucleotide” can also include nucleic acid analogs, polynucleotide analogs, and oligonucleotide analogs. The terms “nucleic acid analog”, “polynucleotide analog” and “oligonucleotide analog” are used interchangeably and, as used herein, refer to a nucleic acid that contains a nucleotide analog or a phosphate ester analog or a pentose sugar analog. Also included within the definition of nucleic acid analogs are nucleic acids in which the phosphate ester or sugar phosphate ester linkages are replaced with other types of linkages, such as N-(2-aminoethyl)-glycine amides and other amides (see, e.g., Nielsen et al., 1991, Science 254: 1497-1500; PCT Publication No. WO 92/20702; U.S. Pat. Nos. 5,719,262 and 5,698,685;); morpholinos (see, e.g., U.S. Pat. No. 5,698,685; U.S. Pat. No. 5,378,841; U.S. Pat. No. 5,185,144); carbamates (see, e.g., Stirchak & Summerton, J. Org. Chem. 52: 4202, 1987); methylene(methylimino) (see, e.g., Vasseur et al., J. Am. Chem. Soc. 114:4006, 1992); 3′-thioformacetals (see, e.g., Jones et al., 1993, J. Org. Chem. 58: 2983); sulfamates (see, e.g., U.S. Pat. No. 5,470,967); 2-aminoethylglycine, commonly referred to as PNA (see, e.g., PCT Publication No. WO 92/20702; Nielsen, Science 254:1497-1500, 1991); and others (see, e.g., U.S. Pat. No. 5,817,781; Frier & Altman, Nucl. Acids Res. 25:4429, 1997 and the references cited therein). Phosphate ester analogs include, but are not limited to, (i) C₁-C₄ alkylphosphonate, e.g. methylphosphonate; (ii) phosphoramidate; (iii) C₁-C₆ alkyl-phosphotriester; (iv) phosphorothioate; and (v) phosphorodithioate. See also, Scheit, Nucleotide Analogs, John Wiley, New York, (1980); Englisch, Agnew. Chem. Int. Ed. Engl. 30:613-29, 1991; Agarwal, Protocols for Polynucleotides and Analogs, Humana Press, 1994; and S. Verma and F. Eckstein, Ann. Rev. Biochem. 67:99-134, 1999.

The term “reporter group” is used in a broad sense herein and refers to any identifiable tag, label, or moiety. The skilled artisan will appreciate that many different species of reporter groups can be used in the present teachings, either individually or in combination with one or more different reporter group. The term reporter group also encompasses an element of multi-element indirect reporter systems, including without limitation, affinity tags; and multi-element interacting reporter groups or reporter group pairs, such as fluorescent reporter group-quencher pairs, including without limitation, pairs comprising fluorescent quenchers and dark quenchers, also known as non-fluorescent quenchers (NFQ).

The term “threshold cycle” or “CT” is used in reference to quantitative or real-time analysis methods and indicates the fractional cycle number at which the amount of analyte, for purposes of the current teachings, Amplicons or their surrogates and including without limitation, one or both strands of any of these, reaches a fixed threshold or limit. Thresholds can be manually set by the user or determined by the software of a real-time instrument. Exemplary real-time instruments include, the ABI PRISM® 7000 Sequence Detection System, the ABI PRISM® 7700 Sequence Detection System, the ABI PRISM® 7900HT Sequence Detection System, the ABI PRISM® 7300 Real-Time PCR System (Applied Biosystems), the Smart Cycler System (Cepheid, distributed by Fisher Scientific), the LightCycler™ System (Roche Molecular), and the Mx4000 (Stratagene, La Jolla, Calif.). Descriptions of threshold cycles and their use, including without limitation ΔC_{T} and ΔΔC_{T}, can be found in, among other places, the ABI PRISM® 7700 Sequence Detection System User Bulletin #2, 2001. In certain embodiments, such real-time quantitation comprises reporter probes, including without limitation, conventional reporter probes and the reporter probes of the present teachings, intercalating dyes, including without limitation, ethidium bromide and SYBR Green I or its equivalent, or such reporter probes and intercalating dyes. Descriptions of real-time analysis can be found in, among other places, Essentials of Real Time PCR, Applied Biosystems P/N 105622, 2002; PCR: The Basics from background to bench, McPherson and Moller, Bios Scientific Publishers Limited, Oxford UK, 2000 (“PCR: The Basics”), particularly at Section 3.3; Real-Time PCR: An Essential Guide, Edwards et al., eds., Horizon Bioscience, Norwich, UK; and Handbook of Fluorescent Probes and Research Products, 9^{th} ed., R. Haugland, Molecular Probes, Inc., 2002 (“Molecular Probes Handbook”), particularly at Section 8.3.

The term “first product” refers to the nucleotide sequence that results when the reverse primer of the first primer set, hybridized to the second region of the corresponding target nucleotide, is extended by an extending enzyme in a primer extension reaction (see, e.g., FIG. 1A). When the target polynucleotide is an RNA molecule, for example but not limited to, a small RNA molecule, the first product can be referred to as a reverse-transcribed product. Those in the art will appreciate that the generation of first products according to the current teachings are at least similar to generating reverse transcripts in conventional RT-PCR techniques.

The interchangeable terms “surrogate” and “Amplicon surrogate” are used in a broad sense herein and refer to any molecule or entity that serves in place of the corresponding Amplicon. In certain embodiments, a surrogate is detected, indicating that the corresponding Amplicon was generated and was or is present. For example but not limited to, a reporter group that was cleaved from a TaqMan® probe during a nuclease assay can be detected and thus indicates that the Amplicon to which the reporter probe hybridized is present. Exemplary Amplicon surrogates include a hybridized reporter probe producing fluorescence or enhanced/altered fluorescence (as appropriate) or at least a part of a reporter probe, for example but not limited to a fluorescent moiety that was cleaved from a nuclease probe or a nuclease probe fragment from which the quencher has been cleaved; a hybridization tag complement that was once annealed to an Amplicon, but was subsequently released by design, including without limitation a ZipChute™ reagent (see, e.g., Rosenblum et al., Published PCT Application WO 2004/046344); a biotinylated fluorophore that was released by design from an Amplicon comprising an CaptAvidin affinity tag; and a single strand of a double-stranded Amplicon, such as a second amplicon. It is to be appreciated that the terms “detecting an Amplicon”, “detecting the second amplicon”, and the like, encompass those situations where a surrogate of that amplicon is detected. For example but not limited to, when detection comprises real time detection of a reporter probe, including without limitation, a TaqMan® assay performed in an ABI PRISM 7700 Sequence Detection System, detection of fluorescence due to the cleavage of a particular TaqMan® probe that is specific for a particular second amplicon is, for purposes of this application, detecting the second amplicon that corresponds to that TaqMan® probe.

As used herein, the term “target-binding portion” refers to the sequence of a forward primer that is the same as the first region of the corresponding target or that sequence of a reverse primer that is complementary to the second region of the corresponding target. Those in the art will appreciate that when the target is a polynucleotide, the term “polynucleotide-binding portion” is interchangeable with the term target-binding portion and when the target is a small RNA molecule, the term “small RNA molecule-binding portion” is interchangeable with the term target-binding portion. Thus, the terms target-binding portion, polynucleotide binding portion, and small RNA molecule-binding portion are used in reference to target sequences in general, polynucleotide targets, and small RNA molecule targets, respectively. The term “primer-binding portion” refers to that sequence of the forward or reverse primers of a first primer set to which the corresponding primers of the second primer set specifically hybridize. Typically, the primers of the second primer set are employed to enable the first product, the first amplicon, the additional first amplicon, or combinations thereof, to be amplified, including without limitation techniques comprising multiple amplification cycles such as PCR. In certain embodiments, a primer of a second primer set is utilized to amplify the corresponding first product, a strand of a corresponding first amplicon, a strand of the corresponding additional first amplicon, a strand of a corresponding second amplicon, or combinations thereof.

The terms “universal base” or “universal nucleotide” are generally used interchangeably herein and refer to a nucleotide analog (including nucleoside analogs) that can substitute for more than one of the natural nucleotides or natural bases in oligonucleotides. Universal bases typically contain an aromatic ring moiety that may or may not contain nitrogen atoms and generally use aromatic ring stacking to stabilize a duplex. In certain embodiments, a universal base may be covalently attached to the C-1′ carbon of a pentose sugar to make a universal nucleotide. In certain embodiments, a universal base does not hydrogen bond specifically with another nucleotide base. In certain embodiments, a nucleotide base may interact with adjacent nucleotide bases on the same nucleic acid strand by hydrophobic stacking. Universal nucleotides and universal bases include, but are not limited to, deoxy-7-azaindole triphosphate (d7AITP), deoxyisocarbostyril triphosphate (dICSTP), deoxypropynylisocarbostyril triphosphate (dPICSTP), deoxymethyl-7-azaindole triphosphate (dM7AITP), deoxylmPy triphosphate (dImPyTP), deoxyPP triphosphate (dPPTP), deoxypropynyl-7-azaindole triphosphate (dP7AITP), 3-methyl isocarbostyril (MICS), 5-methyl isocarbyl (5MICS), imidazole4-carboxamide, 3-nitropyrrole, 5-nitroindole, hypoxanthine, inosine, deoxyinosine, 5-fluorodeoxyuridine, 4-nitrobenzimidizole, and PNA-bases, including pcPNA bases. Descriptions of universal bases can be found in, among other places, Loakes, Nucl. Acids Res. 29:2437-47, 2001; Berger et al., Nucl. Acids Res. 28:2911-14, 2000; Loakes et al., J. Mol. Biol. 270:426-35, 1997; Verma and Eckstein, Ann. Rev. Biochem. 67:99-134, 1998; Published. PCT Application No. US02/33619, and U.S. Pat. Nos. 6,433,134 and 6,433,134.

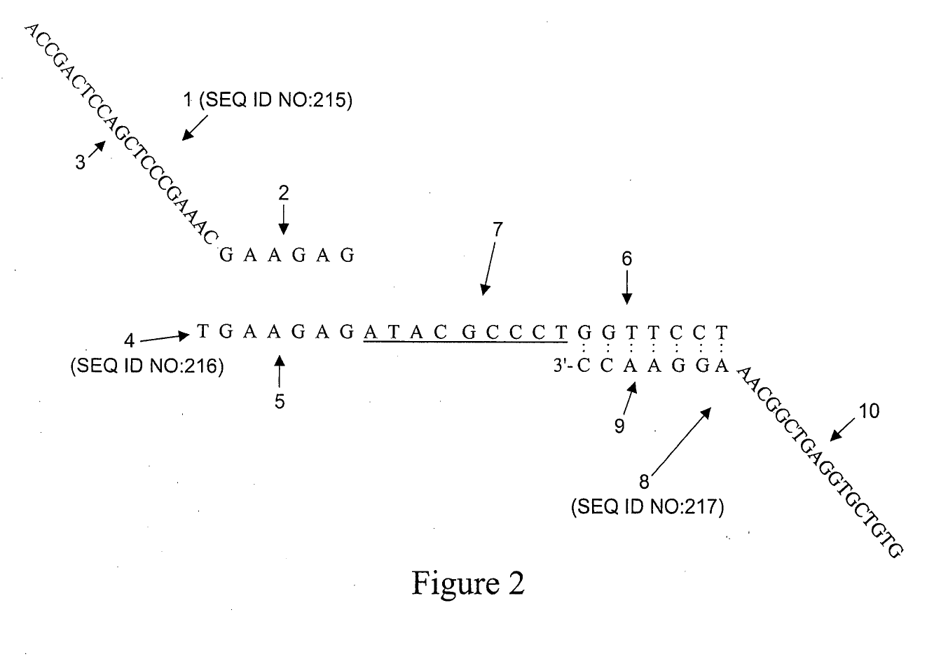

The terms “polynucleotide target”, “target polynucleotide”, or “target” refers to the nucleic acid sequence whose identity, presence, absence, and/or quantity is being evaluated using the methods and kits of the present teachings. In certain embodiments, the target sequence comprises a polynucleotide, which may or may not comprise a deoxyribonucleotide, or an RNA molecule such as a miRNA precursor, including without limitation, a pri-miRNA, a pre-miRNA, or a pri-miRNA and a pre-miRNA. In some embodiments, the polynucleotide target comprises a small RNA molecule, including without limitation, a miRNA, a siRNA, a stRNA, a snoRNA, other ncRNA, and the like. Those in the art will appreciate that when the target polynucleotide is 17-29 nucleotides long, for example but not limited to certain small RNA molecules, there may be, and typically are, nucleotides in the target that are not located within either the first target region or the second target region, that is, they are not the same as the target-binding portion of the corresponding forward primer nor are they complementary to the target-binding portion of the corresponding reverse primer. Such nucleotides may be located in the target sequence 5′ to the first region (see, e.g., FIG. 2); 3′ to the second region; or between the first region and the second region, in which case they are referred to as the “gap” or “gap sequences”. For illustration purposes but not as a limitation, given a miRNA target that is 23 nucleotides long, a corresponding forward primer with a target-binding portion that is seven nucleotides long, and a corresponding reverse primer with a target-binding portion that is eight nucleotides long, there are eight nucleotides in the gap between the first and second regions of the target. FIG. 2 depicts an exemplary gap sequence (7) of nine nucleotides (shown underlined) located between the first region (5; GMGAG) and the second region (6; GGTTCCT) of the target polynucleotide (4). Such gap sequences can be significant in the design of certain reporter probes of the current teachings, as described below, and also in minimizing and/or eliminating the effects of certain “primer dimer” artifacts.

### II. Reagents

The term “reporter probe” refers to a sequence of nucleotides, nucleotide analogs, or nucleotides and nucleotide analogs, that binds to or anneals with an Amplicon, an Amplicon surrogate, or combinations thereof, and when detected, including but not limited to a change in intensity or of emitted wavelength, is used to identify and/or quantify the corresponding target polynucleotide. Most reporter probes can be categorized based on their mode of action, for example but not limited to: nuclease probes, including without limitation TaqMan® probes (see, e.g., Livak, Genetic Analysis: Biomolecular Engineering 14:143-149, 1999; Yeung et al., BioTechniques 36:266-75, 2004); extension probes such as scorpion primers, Lux™ primers, Amplifluors, and the like; hybridization probes such as molecular beacons, Eclipse probes, light-up probes, pairs of singly-labeled reporter probes, hybridization probe pairs, and the like; or combinations thereof. In certain embodiments, reporter probes comprise an amide bond, an LNA, a universal base, or combinations thereof, and include stem-loop and stem-less reporter probe configurations. Certain reporter probes are singly-labeled, while other reporter probes are doubly-labeled. Dual probe systems that comprise FRET between adjacently hybridized probes are within the intended scope of the term reporter probe.

In certain embodiments, a reporter probe comprises a fluorescent reporter group, a quencher reporter group (including without limitation dark quenchers and fluorescent quenchers), an affinity tag, a hybridization tag, a hybridization tag complement, or combinations thereof. In certain embodiments, a reporter probe comprising a hybridization tag complement anneals with the corresponding hybridization tag, a member of a multi-component reporter group binds to a reporter probe comprising the corresponding member of the multi-component reporter group, or combinations thereof. Exemplary reporter probes include TaqMan® probes; Scorpion probes (also referred to as scorpion primers); Lux™ primers; FRET primers; Eclipse probes; molecular beacons, including but not limited to FRET-based molecular beacons, multicolor molecular beacons, aptamer beacons, PNA beacons, and antibody beacons; reporter group-labeled PNA clamps, reporter group-labeled PNA openers, reporter group-labeled LNA probes, and probes comprising nanocrystals, metallic nanoparticles and similar hybrid probes (see, e.g., Dubertret et al., Nature Biotech. 19:365-70, 2001; Zelphati et al., BioTechniques 28:304-15, 2000). In certain embodiments, reporter probes further comprise groove binders including but not limited to TaqMan®MGB probes and TaqMan®MGB-NFQ probes (both from Applied Biosystems). In certain embodiments, reporter probe detection comprises fluorescence polarization detection (see, e.g., Simeonov and Nikiforov, Nucl. Acids Res. 30:e91, 2002).

In addition to such conventional reporter probes, the reporter probes of the current teachings, can be used in the detection, identification, and quantitation of corresponding target polynucleotides. The reporter probes of the current teachings include gap probes, certain chimeric probes, and gap probes that comprise chimeric sequences. Gap probes are designed to specifically hybridize with sequences in Amplicons that are the counterpart of the gap sequences of small RNA molecules, i.e., that sequence in a small RNA molecule that is not the same sequence as the target-binding portion of the corresponding forward primer nor is it complementary to the target-binding portion of the corresponding reverse primer, but are located between these sequences. The reporter probes of the current teachings include: (i) homopolymer probes and also (ii) heteropolymer or chimeric probes. Exemplary homopolymer probes of the current teachings include without limitation, DNA probes, RNA probes, LNA probes, 2′ O-alkyl nucleotide probes, phosphoroamidite probes (for example but not limited to, N3′-P5′ phosphoroamidite probes and morpholino phosphoroamidite probes), 2′-fluoro-arabino nucleic acid (FANA) probes, cyclohexene nucleic acid (CeNA) probes, tricycle-DNA (tcDNA) probes, and PNA probes (see, e.g., Kurreck, Eur. J. Biochem., 270:1628-44, 2003). The chimeric probes of the current teachings, include without limitation, DNA-PNA chimeric probes, DNA-LNA chimeric probes, DNA-2′ O-alkyl chimeric probes, and so forth. In certain embodiments, such DNA chimeric probes comprise at least two deoxyribonucleotides that are usually located at the 5′-end of the probe, but not always.

The reporter probes of the current teachings further comprise a reporter group, and in certain embodiments, comprise a fluorescent reporter group-quencher pair. In certain embodiments, reporter probes are designed to hybridize only with the gap sequences or the complement of gap sequences found in Amplicons. Those in the art will appreciate that even in the presence of “primer dimer” artifacts, which sometimes accompany certain amplification techniques and which may contain some sequences in common with the target polynucleotide, only bona fide Amplicons will contain gap sequences or their complement and thus can stably hybridize with the disclosed reporter probes that hybridize only to the gap (assuming appropriate stringency conditions which those in the art understand can be calculated using various well-known algorithms or determined empirically). In certain embodiments, the Amplicon-binding portion of a reporter probe is designed to hybridize with the gap sequences or the gap sequence complements found in Amplicons and also to a few nucleotides adjacent to the gap sequences, typically one or two additional nucleotides on one or both sides of the Amplicon gap sequences.

In certain embodiments, chimeric reporter probes are disclosed that comprise a reporter group, two or more deoxyribonucleotides, and downstream, a multiplicity of nucleotide analogs. Typically such nucleotide analogs are selected because they do not readily serve as templates for DNA polymerases or reverse transcriptases and thus are not amplified during primer extension reactions. Exemplary non-extendable nucleotide analogs include without limitation, locked nucleic acids (LNAs), peptide nucleic acids (PNAs), and 2′ O-alkyl nucleotides, for example but not limited to, 2′ O-methyl nucleotides and 2′ O-ethyl nucleotides. In certain embodiments, chimeric reporter probes comprise a reporter group and at least two deoxyribonucleotides located upstream from at least four PNAs. In certain embodiments, a chimeric reporter probe comprises a fluorescent reporter group-quencher pair. In certain embodiments, a fluorescent reporter group is located upstream from at least two deoxyribonucleotides or is attached to at least one of the two deoxyribonucleotides, and the quencher is located downstream (or vice versa) to form a fluorescent reporter group-quencher pair, which may or may not comprise fluorescence resonance energy transfer (FRET). Those in the art will appreciate that such reporter probes can be particularly useful for certain detection techniques, such as nuclease assays, including without limitation, TaqMan® assays.

In certain embodiments, a reporter probe does not comprise a sequence that is the same as or is complementary to the target-binding portions of either forward primer or reverse primer of a first primer set. In certain embodiments, a reporter probe comprises: (a) a nucleotide or nucleotide analog that has the same nucleotide base as the 3′-end of the polynucleotide-binding portion of the forward primer or is complementary to the 3′-end of the target-binding portion of the forward primer; adjacent to (b) at least two nucleotides or nucleotide analogs that have the same nucleotide bases as or are complementary to at least two nucleotides of the target polynucleotide and that are not the same as or complementary to the polynucleotide-binding portion of the forward primer or the polynucleotide-binding portion of the reverse primer; adjacent to (c) a nucleotide or nucleotide analog that has the same nucleotide base as the 3′-end of the polynucleotide-binding portion of the reverse primer or is complementary to the 3′-end of the polynucleotide-binding portion of the reverse primer.

The disclosed first primer sets include forward primers and reverse primers, each comprising unusually short target-binding portions, i.e., forward primers with no more than ten nucleotides that have the same sequence as the first target region and reverse primers with no more than ten nucleotides that are complementary to the second target region. In certain embodiments, the target-binding portion of the forward primers contain six, seven, eight, or nine nucleotides that have the same sequence as the corresponding first region of the target. In certain embodiments, the target-binding portion of the reverse primers contain six, seven, eight, or nine nucleotides that are complementary to the corresponding second region of the target. In certain embodiments, the forward primers and the reverse primers further comprise an additional portion that is upstream from the target-binding portion and can, but need not be, a primer-binding portion. When present, such primer-binding portions are designed to selectively hybridize with the respective primers of the corresponding second primer set. Thus, when incorporated in Amplicons, additional amplification is possible using the corresponding second primer set and an appropriate extending enzyme.

For illustration purposes and not as a limitation, an illustrative first primer set is depicted in FIG. 2. The forward primer (1) comprises a target-binding portion (2) that has the same sequence as the first region (5; GAAGAG) of the corresponding target (4) and an optional upstream portion (3). The downstream primer (8) of the exemplary first primer set comprises a target-binding portion (9) that is complementary to and is shown hybridized with the second region (6) of the corresponding target (4) and an optional second portion (10) that is upstream from the target-binding portion (9). Either or both of the optional second portions (3, 10) of a first primer set can, but need not, serve as a hybridization tag; a primer-binding portion to which a primer of the second primer set can anneal; an attachment site for an affinity tag; cross-linker, such as but not limited to, a linker arm; and the like. In certain embodiments, the target-binding portion and the second portion of a forward primer, a reverse primer, or both may overlap, at least in part. In certain embodiments, the first region or the second region of the target polynucleotide does not comprise the terminal nucleotide of the target (see, e.g., the first target region (5) “GAAGAG” in FIG. 2).

The second primer sets of the current teachings comprise a first primer and a second primer that are designed to anneal to regions of Amplicons that correspond to the primer-binding portions of the forward and reverse primers, respectively, of the corresponding first primer set. In certain embodiments, a primer of a second primer set is a universal primer. In certain embodiments, a second primer set comprises a universal forward primer and a universal reverse primer. In certain embodiments, a primer of a second primer set further comprises a hybridization tag, an affinity tag, a reporter group, or combinations thereof. In certain embodiments, a hybridization tag allows the corresponding Amplicon to be identified. In certain embodiments, a first primer of the second primer set comprises a first universal priming sequence and the second primer of the corresponding second primer set comprises a second universal priming sequence. In certain embodiments, one primer of a second primer set comprises a universal priming sequence and the other primer of the corresponding second primer set comprises a hybridization tag, including without limitation, a unique hybridization tag that can be used to subsequently identify the corresponding Amplicon.

Certain of the disclosed methods comprise a multiplicity of different first primer sets for identifying and/or quantitating a multiplicity of different polynucleotide targets, including without limitation, polynucleotides comprising a deoxyribonucleotide, a miRNA precursor, a small RNA molecule, or combinations thereof. Certain embodiments comprise generating a multiplicity of different first amplicons and a multiplicity of different additional first amplicons. Certain embodiments comprise or further comprise a multiplex reaction wherein a multiplicity of different second amplicons are generated. In certain embodiments, one or more multiplex reactions for generating Amplicons are performed in the same reaction vessel, including without limitation, a multi-well plate, such as a 96-well, a 384-well, a 1536-well plate, and so forth; or a microfluidic device, for example but not limited to, a TaqMan® Low Density Array (Applied Biosystems). In certain embodiments, a multiplicity of different second amplicons are generated in different wells or chambers of the same reaction vessel, and a subset of the multiplicity of different second amplicons are generated and detected in at least two different wells or chambers. Typically this occurs in a series of parallel single-plex, two-plex, three-plex, or four-plex reactions, although higher levels of parallel multiplexing are also within the intended scope of the current teachings. In certain embodiments (i) the steps for generating a first product, a first amplicon, an additional first amplicon, or combinations thereof, (ii) the steps for generating second amplicons, or (iii) both, are automated or semi-automated, using an instrument, including without limitation, a thermocycler or a real-time instrument, software, robotics, or combinations thereof.

The binding portions of the first primer set primers, the second primer set primers, and the reporter probes of the current teachings are of sufficient length to permit specific annealing to complementary regions of corresponding target sequences, corresponding Amplicons, or combinations thereof, as appropriate. The criteria for designing sequence-specific nucleic acid primers and reporter probes are well known to those in the art. Detailed descriptions of nucleic acid primer and reporter probe design can be found in, among other places, Diffenbach and Dveksler, PCR Primer, A Laboratory Manual, Cold Spring Harbor Press (1995); R. Rapley, The Nucleic Acid Protocols Handbook (2000), Humana Press, Totowa, N.J. (“Rapley”); Schena; and Kwok et al., Nucl. Acid Res. 18:999-1005 (1990). Primer and reporter probe design software programs are also commercially available, including without limitation, Primer Express, Applied Biosystems; Primer Premier and Beacon Designer software, PREMIER Biosoft International, Palo Alto, Calif.; Primer Designer 4, Sci-Ed Software, Durham, N.C.; Primer Detective, ClonTech, Palo Alto, Calif.; Lasergene, DNASTAR, Inc., Madison, Wis.; Oligo software, National Biosciences, Inc., Plymouth, Minn.; iOligo, Caesar Software, Portsmouth, N.H.; and RTPrimerDB on the world wide web at realtimeprimerdatabase.ht.st or at medgen31.urgent.be/primerdatabase/index (see also, Pattyn et al., Nucl. Acid Res. 31:122-23, 2003).

Those in the art understand that primers and reporter probes suitable for use with the disclosed methods and kits can be identified empirically using the current teachings and routine methods known in the art, without undue experimentation. For example, suitable primers, primer sets, and reporter probes can be obtained by selecting candidate target polynucleotides from the relevant scientific literature, including but not limited to, appropriate databases and using computational algorithms (see, e.g., miRNA Registry, on the world-wide web at sanger-ac.uk/Software/Rfam/miRNA/index; MiRscan, available on the web at genes/mit.edu/mirscan; miRseeker; and Carter et al., Nucl. Acids Res. 29(19):3928-38, 2001). When polynucleotides of interest are identified, test primers and/or reporter probes can be synthesized using well known synthesis techniques and their suitability can be evaluated in the disclosed methods and kits (see, e.g., Current Protocols in Nucleic Acid Chemistry, Beaucage et al., eds., John Wiley & Sons, New York, N.Y., including updates through August 2004 (“Beaucage et al.”); Blackburn and Gait; Glen Research 2002 Catalog, Sterling, Va.; The Glen Report 16(2):5, 2003, Glen Research; Synthetic Medicinal Chemistry 2003/2004, Berry and Associates, Dexter, Mich.; and PNA Chemistry for the Expedite™ 8900 Nucleic Acid Synthesis System User's Guide, Applied Biosystem). Those in the art will appreciate that the melting temperature (Tₘ) of a primer or reporter probe can be increased by, among other things, incorporating a minor groove binder, substituting a an appropriate nucleotide analog for a nucleotide (i.e., a chimeric probe), or using a homopolymer probe comprising appropriate analogs, including without limitation, a PNA oligomer probe or an LNA oligomer probe, with or without a groove binder.

In certain embodiments, a multiplicity of primers, a multiplicity of Amplicons, a multiplicity of Amplicon surrogates, or combinations thereof, have substantially similar distinctive mobilities, for example but not limited to, when a multiplicity of elements comprising mobility modifiers have substantially similar distinctive mobilities so they can be bulk separated or they can be separated from other elements comprising mobility modifiers with different distinctive mobilities. In certain embodiments, a multiplicity of primers comprising mobility modifiers, a multiplicity of first products comprising mobility modifiers, a multiplicity of Amplicons comprising mobility modifiers, a multiplicity of Amplicon surrogates, or combinations thereof, have different distinctive mobilities.

In certain embodiments, a mobility modifier comprises a nucleotide polymer chain, including without limitation, an oligonucleotide polymer chain or a polynucleotide polymer chain. For example but not limited to, a series of additional non-target sequence-specific nucleotides or nucleotide spacers in a primer, hybridization tag complement, reporter probe, or the like (see e.g., Tong et al., Nat. Biotech. 19:756-759 (2001)). In certain embodiments, a mobility modifier comprises a non-nucleotide polymer chain. Exemplary non-nucleotide polymer chains include, without limitation, peptides, polypeptides, polyethylene oxide (PEO), or the like. In certain embodiments, a polymer chain comprises a substantially uncharged, water-soluble chain, such as a chain composed of a PEO unit; a polypeptide chain; or combinations thereof.

The polymer chain can comprise a homopolymer, a random copolymer, a block copolymer, or combinations thereof. Furthermore, the polymer chain can have a linear architecture, a comb architecture, a branched architecture, a dendritic architecture (e.g., polymers containing polyamidoamine branched polymers, Polysciences, Inc. Warrington, Pa.), or combinations thereof. In certain embodiments, a polymer chain is hydrophilic, or at least sufficiently hydrophilic when hybridized or bound to an element to ensure that the element-mobility modifier is readily soluble in aqueous medium. Where the mobility-dependent analytical technique is electrophoresis, in certain embodiments, the polymer chains are uncharged or have a charge/subunit density that is substantially less than that of its corresponding element.

The synthesis of polymer chains useful as mobility modifiers will depend, at least in part, on the nature of the polymer. Methods for preparing suitable polymers generally follow well-known polymer subunit synthesis methods. These methods, which involve coupling of defined-size, multi-subunit polymer units to one another, either directly or through charged or uncharged linking groups, are generally applicable to a wide variety of polymers, such as PEO, polyglycolic acid, polylactic acid, polyurethane polymers, polypeptides, oligosaccharides, and nucleotide polymers. Such methods of polymer unit coupling are also suitable for synthesizing selected-length copolymers, e.g., copolymers of PEO units alternating with polypropylene units. Polypeptides of selected lengths and amino acid composition, either homopolymer or mixed polymer, can be synthesized by standard solid-phase methods (see, e.g., Int. J. Peptide Protein Res., 35: 161-214, 1990).

One method for preparing PEO polymer chains having a selected number of hexaethylene oxide (HEO) units, an HEO unit is protected at one end with dimethoxytrityl (DMT), and activated at its other end with methane sulfonate. The activated HEO is then reacted with a second DMT-protected HEO group to form a DMT-protected HEO dimer. This unit-addition is then carried out successively until a desired PEO chain length is achieved (see, e.g., U.S. Pat. No. 4,914,210; see also, U.S. Pat. No. 5,777,096).

The term “extending enzyme” refers to a polypeptide that is able to catalyze the 5′-3′ extension of a hybridized primer in template-dependent manner under suitable reaction conditions including without limitation, appropriate nucleotide triphosphates, cofactors, buffer, and the like. Extending enzymes are typically DNA polymerases, for example but not limited to, RNA-dependent DNA polymerases, including without limitation reverse transcriptases, DNA-dependent DNA polymerases, and include DNA polymerases that, at least under certain conditions, share properties of both of these classes of DNA polymerases, including enzymatically active mutants or variants of each of these. In certain embodiments, an extending enzyme is a reverse transcriptase, including enzymatically active mutants or variants thereof, for example but not limited to, retroviral reverse transcriptases such as Avian Myeloblastosis Virus (AMV) reverse transcriptase and Moloney Murine Leukemia Virus (MMLV) reverse transcriptase. In certain embodiments, an extending enzyme is a DNA polymerase, including enzymatically active mutants or variants thereof. Certain DNA polymerases possess reverse transcriptase activity under some conditions, for example but not limited to, the DNA polymerase of Thermus thermophilus (Tth DNA polymerase, E.C. 2.7.7.7) which demonstrates reverse transcription in the presence of Mn²⁺, but not Mg²⁺ (see also, GeneAmp® AccuRT RNA PCR Kit and Hot Start RNA PCR Kit comprising a recombinant polymerase derived from Thermus specie Z05, both from Applied Biosystems). Likewise, certain reverse transcriptases possess DNA polymerase activity under certain reaction conditions, including without limitation, AMV reverse transcriptase and MMLV reverse transcriptase. Descriptions of appropriate DNA polymerases for use with the disclosed methods and kits can be found in, among other places, Lehninger Principles of Biochemistry, 3d ed., Nelson and Cox, Worth Publishing, New York, N.Y., 2000 (“Lehninger”), particularly Chapters 26 and 29; R. M. Twyman, Advanced Molecular Biology: A Concise Reference. Bios Scientific Publishers, New York, N.Y. (1999); and Enzymatic Resource Guide: Polymerases, Promega, Madison, Wis. (1998). Expressly within the intended scope of the term extending enzyme are enzymatically active mutants or variants thereof, as are enzymes modified to confer different temperature-sensitive properties (see, e.g., U.S. Pat. Nos. 5,773,258; 5,677,152; and 6,183,998).

In certain embodiments, a primer, an Amplicon, or a primer and an Amplicon comprise a reporter group. In certain embodiments, a primer comprising a reporter group is incorporated into an Amplicon by primer extension. In certain embodiments, an Amplicon comprises a reporter group that was incorporated into the Amplicon when a reporter group-labeled dNTP was incorporated during primer extension or other amplification technique. A reporter group can, under appropriate conditions, emit a fluorescent, a chemiluminescent, a bioluminescent, a phosphorescent, or an electrochemiluminescent signal. Exemplary reporter groups include, but are not limited to fluorophores, radioisotopes, chromogens, enzymes, antigens including but not limited to epitope tags, semiconductor nanocrystals such as quantum dots, heavy metals, dyes, phosphorescence groups, chemiluminescent groups, electrochemical detection moieties, affinity tags, binding proteins, phosphors, rare earth chelates, transition metal chelates, near-infrared dyes, including but not limited to, “Cy.7.SPh.NCS,” “Cy.7.OphEt.NCS,” “Cy7.OphEt.CO₂Su”, and IRD800 (see, e.g., J. Flanagan et al., Bioconjug. Chem. 8:751-56 (1997); and DNA Synthesis with IRD800 Phosphoramidite, LI-COR Bulletin #111, LI-COR, Inc., Lincoln, Nebr.), electrochemiluminescence labels, including but not limited to, tris(bipyridal) ruthenium (II), also known as Ru(bpy)₃²⁺, Os(1,10-phenanthroline)₂bis(diphenylphosphino)ethane²+, also known as Os(phen)₂(dppene)²⁺, luminol/hydrogen peroxide, Al(hydroxyquinoline-5-sulfonic acid), 9,10-diphenylanthracene-2-sulfonate, and tris(4-vinyl-4′-methyl-2,2′-bipyridal) ruthenium (II), also known as Ru(v-bpy₃²⁺), and the like.

The term reporter group also encompasses an element of multi-element indirect reporter systems, including without limitation, affinity tags such as biotin:avidin, antibody:antigen, ligand:receptor including but not limited to binding proteins and their ligands, and the like, in which one element interacts with one or more other elements of the system in order to effect the potential for a detectable signal. Exemplary multi-element reporter systems include an oligonucleotide comprising a biotin reporter group and a streptavidin-conjugated fluorophore, or vice versa; an oligonucleotide comprising a DNP reporter group and a fluorophore-labeled anti-DNP antibody; and the like. In certain embodiments, reporter groups, particularly multi-element reporter groups, are not necessarily used for detection, but serve as affinity tags for isolation/separation, for example but not limited to, a biotin reporter group and a streptavidin-coated Substrate, or vice versa; a digoxygenin reporter group and a substrate comprising an anti-digoxygenin antibody or a digoxygenin-binding aptamer; a DNP reporter group and a Substrate comprising an anti-DNP antibody or a DNP-binding aptamer; and the like. Detailed protocols for attaching reporter groups to oligonucleotides, polynucleotides, peptides, antibodies and other proteins, mono-, di- and oligosaccharides, organic molecules, and the like can be found in, among other places, G. T. Hermanson, Bioconjugate Techniques, Academic Press, San Diego, 1996; Beaucage et al.; Molecular Probes Handbook; and Pierce Applications Handbook and Catalog 2003-2004, Pierce Biotechnology, Rockford, Ill., 2003 (“Pierce Applications Handbook”).

Multi-element interacting reporter groups are also within the scope of the term reporter group, such as fluorophore-quencher pairs, including without limitation fluorescent quenchers and dark quenchers (also known as non-fluorescent quenchers). A fluorescent quencher can absorb the fluorescent signal emitted from a fluorophore and after absorbing enough fluorescent energy, the fluorescent quencher can emit fluorescence at a characteristic wavelength, e.g., fluorescent resonance energy transfer. For example without limitation, the FAM-TAMRA pair can be illuminated at 492 nm, the excitation peak for FAM, and emit fluorescence at 580 nm, the emission peak for TAMRA. A dark quencher, appropriately paired with a fluorescent reporter group, absorbs the fluorescent energy from the fluorophore, but does not itself fluoresce. Rather, the dark quencher dissipates the absorbed energy, typically as heat. Exemplary dark or nonfluorescent quenchers include Dabcyl, Black Hole Quenchers, Iowa Black, QSY-7, AbsoluteQuencher, Eclipse non-fluorescent quencher, metal clusters such as gold nanoparticles, and the like. Certain dual-labeled probes comprising fluorophore-quencher pairs can emit fluorescence when the members of the pair are physically separated, for example but without limitation, nuclease probes such as TaqMan® probes. Other dual-labeled probes comprising fluorophore-quencher pairs can emit fluorescence when the members of the pair are spatially separated, for example but not limited to hybridization probes, such as molecular beacons, or extension probes, such as Scorpion primers. Fluorophore-quencher pairs are well known in the art and used extensively for a variety of reporter probes (see, e.g., Yeung et al., BioTechniques 36:266-75, 2004; Dubertret et al., Nat. Biotech. 19:365-70, 2001; and Tyagi et al., Nat. Biotech. 18:1191-96, 2000).

In certain embodiments, a reporter group comprises an electrochemiluminescent moiety that can, under appropriate conditions, emit detectable electrogenerated chemiluminescence (ECL). In ECL, excitation of the electrochemiluminescent moiety is electrochemically driven and the chemiluminescent emission can be optically detected. Exemplary electrochemiluminescent reporter group species include: Ru(bpy)₃²⁺ and Ru(v-bpy)₃²⁺ with emission wavelengths of 620 nm; Os(phen)₂(dppene)²⁺ with an emission wavelength of 584 nm; luminol/hydrogen peroxide with an emission wavelength of 425 nm; AI(hydroxyquinoline-5-sulfonic acid) with an emission wavelength of 499 nm; and 9,10-diphenylanothracene-2-sulfonate with an emission wavelength of 428 nm; and the like. Forms of these three electrochemiluminescent reporter group species that are modified to be amenable to incorporation into probes are commercially available or can be synthesized without undue experimentation using techniques known in the art. For example, a Ru(bpy)₃²⁺ N-hydroxy succinimide ester for coupling to nucleic acid sequences through an amino linker group has been described (see, U.S. Pat. No. 6,048,687); and succinimide esters of Os(phen)₂(dppene)²⁺ and Al(HQS)₃³⁺ can be synthesized and attached to nucleic acid sequences using similar methods. The Ru(bpy)₃²⁺ electrochemiluminescent reporter group can be synthetically incorporated into nucleic acid sequences using commercially available ru-phosphoramidite (IGEN International, Inc., Gaithersburg, Md.) (see, e.g., Osiowy, J. Clin. Micro. 40:2566-71, 2002).

Additionally other polyaromatic compounds and chelates of ruthenium, osmium, platinum, palladium, and other transition metals have shown electrochemiluminescent properties. Detailed descriptions of ECL and electrochemiluminescent moieties can be found in, among other places, A. Bard and L. Faulkner, Electrochemical Methods, John Wiley & Sons (2001); M. Collinson and M. Wightman, Anal. Chem. 65:2576 (1993); D. Brunce and M. Richter, Anal. Chem. 74:3157 (2002); A. Knight, Trends in Anal. Chem. 18:47 (1999); B. Muegge et al., Anal. Chem. 75:1102 (2003); H. Abrunda et al., J. Amer. Chem. Soc. 104:2641 (1982); K. Maness et al., J. Amer. Chem. Soc. 118:10609 (1996); M. Collinson and R. Wightman, Science 268:1883 et seq. (1995); and U.S. Pat. No. 6,479,233 (see also, O'Sullivan et al., Nucl. Acids Res. 30:e114, 2002 for a discussion of phosphorescent lanthanide and transition metal reporter groups).

### III. Techniques

A polynucleotide target according to the present teachings may be derived from any living, or once living, organism, including but not limited to, prokaryotes, archaea, viruses, and eukaryotes. The polynucleotide target can also be synthetic. The polynucleotide target may originate from the nucleus, typically genomic DNA (gDNA) and RNA transcription products (including without limitation certain miRNA precursors and other small RNA molecules), or may be extranuclear, e.g., cytoplasmic, plasmid, mitochondrial, viral, etc. The skilled artisan appreciates that gDNA includes not only full length material, but also fragments generated by any number of means, for example but not limited to, enzyme digestion, sonication, shear force, and the like. In certain embodiments, the polynucleotide target may be present in a double-stranded or single-stranded form.

A variety of methods are available for obtaining a polynucleotide target for use with the methods and kits of the present teachings. When the target sequences are obtained from a biological matrix, certain isolation techniques are typically employed, including without limitation, (1) organic extraction followed by ethanol precipitation, e.g., using a phenol/chloroform organic reagent (see, e.g., Ausbel et al., particularly Volume 1, Chapter 2, Section I), in certain embodiments, using an automated extractor, e.g., the Model 341 DNA Extractor (Applied Biosystems); (2) stationary phase adsorption methods (see, e.g., U.S. Pat. No. 5,234,809; Walsh et al., BioTechniques 10(4): 506-513 (1991)); and (3) salt-induced DNA precipitation methods (see, e.g., Miller et al., Nucl. Acids Res. 16(3): 9-10, 1988), such precipitation methods being typically referred to as “salting-out” methods. In certain embodiments, the above isolation methods may be preceded by an enzyme digestion step to help eliminate unwanted protein from the sample, e.g., digestion with proteinase K, or other like proteases. See, e.g., U.S. patent application Ser. No. 09/724,613; see also, U.S. patent application Ser. Nos. 10/618,493 and 10/780,963; and U.S. Provisional Patent Application Ser. Nos. 60/499,082 and 60/523,056. A variety of commercially available kits and instruments can also be used to obtain target polynucleotides, including but not limited to small RNA molecules and their precursors, for example but not limited to, the ABI PRISM® TransPrep System, BloodPrep™ Chemistry, ABI PRISM® 6100 Nucleic Acid PrepStation, and ABI PRISM® 6700 Automated Nucleic Acid Workstation (all from Applied Biosystems); the SV96 Total RNA Isolation System and RNAgents® Total RNA Isolation System (Promega, Madison, Wis.); the mirVana miRNA Isolation Kit (Ambion, Austin, Tex.); and the Absolutely RNA™ Purification Kit and the Micro RNA Isolation Kit (Stratagene, La Jolla, Calif.).

In certain embodiments, nucleic acids in a sample may be subjected to restriction enzyme cleavage and the resulting restriction fragments may be employed as polynucleotide targets. Different polynucleotide targets may be different portions of a single contiguous nucleic acid or may be on different nucleic acids. Different target sequences of a single contiguous nucleic acid may or may not overlap. Certain polynucleotide targets may also be present within other target sequences, including without limitation, primary miRNA (pri-miRNA), precursor miRNA (pre-miRNA), miRNA, mRNA, and siRNA.

Certain embodiments of the disclosed methods comprise a step for generating a first product, a step for generating a first amplicon, a step for generating additional first amplicons, a step for generating second amplicons, a step for generating more second amplicons, or combinations thereof. In certain embodiments, at least some of these steps occur simultaneously or nearly simultaneously in a first reaction composition. In certain embodiments, some of these steps occur in a first reaction composition and other steps occur in a second reaction composition or a third reaction composition. Certain kits of the current teachings comprise an amplification means.

Amplification according to the present teachings encompasses any means by which at least a part of a target polynucleotide and/or an Amplicon is reproduced, typically in a template-dependent manner, including without limitation, a broad range of techniques for amplifying nucleic acid sequences, either linearly or exponentially. Exemplary techniques for performing an amplifying step include the polymerase chain reaction (PCR), primer extension (including but not limited to reverse transcription), strand displacement amplification (SDA), multiple displacement amplification (MDA), nucleic acid strand-based amplification (NASBA), rolling circle amplification (RCA), transcription-mediated amplification (TMA), transcription, and the like, including multiplex versions or combinations thereof. Descriptions of such techniques can be found in, among other places, Sambrook and Russell; Sambrook et al.; Ausbel et al.; PCR Primer: A Laboratory Manual, Diffenbach, Ed., Cold Spring Harbor Press (1995); The Electronic Protocol Book, Chang Bioscience (2002); Msuih et al., J. Clin. Micro. 34:501-07 (1996); Rapley; U.S. Pat. Nos. 6,027,998 and 6,511,810; PCT Publication Nos. WO 97/31256 and WO 01/92579; Ehrlich et al., Science 252:1643-50 (1991); Innis et al., PCR Protocols: A Guide to Methods and Applications, Academic Press (1990); Favis et al., Nature Biotechnology 18:561-64 (2000); and Rabenau et al., Infection 28:97-102 (2000).

In certain embodiments, amplification comprises a cycle of the sequential steps of: (i) hybridizing a primer with a target polynucleotide and/or an Amplicon comprising complementary or substantially complementary sequences; (ii) extending the hybridized primer, thereby synthesizing a strand of nucleotides in a template-dependent manner; and (iii) denaturing the newly-formed nucleic acid duplex to separate the strands. The cycle may or may not be repeated, as desired. Amplification can comprise thermocycling or can be performed isothermally. In certain embodiments, nascent nucleic acid duplexes are not initially denatured, but are used in their double-stranded form in one or more subsequent steps and either one or both strands can, but need be, detected. In certain embodiments, single-stranded Amplicons are generated, for example but not limited to, asymmetric PCR.

Primer extension is an amplifying technique that comprises elongating a primer that is annealed to a template in the 5′=>3′ direction using an amplifying means such as an extending enzyme, for example but not limited to, a DNA polymerase (including without limitation, a reverse transcriptase). According to certain embodiments, with appropriate buffers, salts, pH, temperature, and nucleotide triphosphates, including analogs thereof, an extending enzyme incorporates nucleotides complementary to the template strand starting at the 3′-end of an annealed primer, to generate a complementary strand. In certain embodiments, the extending enzyme used for primer extension lacks or substantially lacks 5′-exonuclease activity.

The skilled artisan will understand that a number of different enzymes, including without limitation, extending enzymes could be used in the disclosed methods and kits, for example but not limited to, those isolated from thermostable or hyperthermostable prokaryotic, eukaryotic, or archaeal organisms. The skilled artisan will also understand that enzymes such as polymerases, including but not limited to DNA-dependent DNA polymerases and RNA-dependent DNA polymerases, include not only naturally occurring enzymes, but also recombinant enzymes; and enzymatically active fragments, cleavage products, mutants, or variants of such enzymes, for example but not limited to Klenow fragment, Stoffel fragment, Taq FS (Applied Biosystems), 9°Nₘ™ DNA Polymerase (New England BioLabs, Beverly, Mass.), and mutant enzymes (including without limitation, naturally-occurring and man-made mutants), described in Luo and Barany, Nucl. Acids Res. 24:3079-3085 (1996), Eis et al., Nature Biotechnol. 19:673-76 (2001), and U.S. Pat. Nos. 6,265,193 and 6,576,453. Reversibly modified polymerases, for example but not limited to those described in U.S. Pat. No. 5,773,258, are also within the scope of the disclosed teachings. The present teachings also contemplate various uracil-based decontamination strategies, wherein for example uracil can be incorporated into an amplification reaction, and subsequent carry-over products removed with various glycosylase treatments (see, e.g., U.S. Pat. No. 5,536,649). Those in the art will understand that any protein with the desired enzymatic activity can be used in the disclosed methods and kits. Descriptions of DNA polymerases, including reverse transcriptases, uracil N-glycosylase, and the like, can be found in, among other places, Twyman, Advanced Molecular Biology, BIOS Scientific Publishers, 1999; Enzyme Resource Guide, rev. 092298, Promega, 1998; Sambrook and Russell; Sambrook et al.; Lehninger; PCR: The Basics; and Ausbel et al.

Certain embodiments of the disclosed methods and kits comprise separating (either as a separate step or as part of a step for detecting) or a separation means. Separating comprises any process that removes at least some unreacted components or at least some reagents from an Amplicon. In certain embodiments, Amplicons are separated from unreacted components and reagents, including without limitation, unreacted molecular species present in a reaction composition, extending enzymes, primers, co-factors, dNTPs, and the like. The skilled artisan will appreciate that a number of well-known separation means can be used in the methods and kits disclosed herein and thus the separation technique employed is not a limitation on the disclosed methods.

Exemplary means/techniques for performing a separation step include gel electrophoresis, for example but not limited to, isoelectric focusing and capillary electrophoresis; dielectrophoresis; flow cytometry, including but not limited to fluorescence-activated sorting techniques using beads, microspheres, or the like; liquid chromatography, including without limitation, HPLC, FPLC, size exclusion (gel filtration) chromatography, affinity chromatography, ion exchange chromatography, hydrophobic interaction chromatography, immunoaffinity chromatography, and reverse phase chromatography; affinity tag binding, such as biotin-avidin, biotin-streptavidin, maltose-maltose binding protein (MBP), and calcium-calcium binding peptide; aptamer-target binding; hybridization tag-hybridization tag complement annealing; mass spectrometry, including without limitation MALDI-TOF, MALDI-TOF-TOF, tandem mass spec (MS-MS), LC-MS, and LC-MS/MS; a microfluidic device; and the like. Discussion of separation techniques and separation-detection techniques, can be found in, among other places, Rapley; Sambrook et al.; Sambrook and Russell; Ausbel et al.; Molecular Probes Handbook; Pierce Applications Handbook; Capillary Electrophoresis: Theory and Practice, P. Grossman and J. Colburn, eds., Academic Press, 1992; The Expanding Role of Mass Spectrometry in Biotechnology, G. Siuzdak, MCC Press, 2003; PCT Publication No. WO 01/92579; and M. Ladisch, Bioseparations Engineering: Principles, Practice, and Economics, John Wiley & Sons, 2001.

In certain embodiments, a separating step comprises binding or annealing an Amplicon, an Amplicon surrogate, or both to a substrate, for example but not limited to binding a double-stranded second amplicon comprising a biotin affinity tag a streptavidin-coated substrate or binding a single-stranded Amplicon comprising a hybridization tag to a substrate comprising a hybridization tag complement at a unique address on the substrate. Suitable substrates include but are not limited to: microarrays, including fixed arrays and bead arrays; appropriately treated or coated reaction vessels and surfaces; beads, for example but not limited to magnetic beads, paramagnetic beads, latex beads, metallic beads, polymer beads, dye-impregnated beads, and coated beads; optically identifiable micro-cylinders; biosensors comprising transducers; and the like (see, e.g., Tong et al., Nat. Biotech. 19:756-59 (2001); Gerry et al., J. Mol. Biol. 292:251-62 (1999); Srisawat et al., Nucl. Acids Res. 29:e4 (2001); Han et al., Nat. Biotech. 19:631-35, 2001; and Stears et al., Nat. Med. 9:140-45, including supplements, 2003). Those in the art will appreciate that any number of substrates may be employed in the disclosed methods and kits and that the shape and composition of the substrate is generally not limiting.

In certain embodiments, an Amplicon or its surrogate is separated by liquid chromatography. Exemplary stationary phase chromatography media for use in the teachings herein include reversed-phase media (e.g., C-18 or C-8 solid phases), ion-exchange media (particularly anion-exchange media), and hydrophobic interaction media. In certain embodiments, an Amplicon or its surrogate is separated by micellar electrokinetic capillary chromatography (MECC).

Reversed-phase chromatography is carried out using an isocratic, or more typically, a linear, curved, or stepped solvent gradient, wherein the level of a nonpolar solvent such as acetonitrile or isopropanol in aqueous solvent is increased during a chromatographic run, causing analytes to elute sequentially according to affinity of each analyte for the solid phase. For separating polynucleotides, including Amplicons and at least some Amplicon surrogates, an ion-pairing agent (e.g., a tetra-alkylammonium) is typically included in the solvent to mask the charge of phosphate.

The mobility of Amplicons can be varied by using mobility modifiers comprising polymer chains that alter the affinity of the element to which it is attached for the solid, or stationary phase. Thus, with reversed phase chromatography, an increased affinity of the Amplicons and/or Amplicon surrogates for the stationary phase can be attained by adding a moderately hydrophobic tail (e.g., PEO-containing polymers, short polypeptides, and the like) to the mobility modifier. Longer tails impart greater affinity for the solid phase, and thus require higher non-polar solvent concentration for the ligation products or ligation product surrogates to be eluted (and a longer elution time).

In certain embodiments, an Amplicon, an Amplicon surrogate, or both, are resolved by electrophoresis in a sieving or non-sieving matrix. In certain embodiments, the electrophoretic separation is carried out in a capillary tube by capillary electrophoresis, including without limitation, microcapillaries and nanocapillaries (see, e.g., Capillary Electrophoresis: Theory and Practice, Grossman and Colburn eds., Academic Press, 1992). Exemplary sieving matrices for use in the disclosed teachings include covalently crosslinked matrices, such as polyacrylamide covalently crosslinked with bis-acrylamide; gel matrices formed with linear polymers (see, e.g., U.S. Pat. No. 5,552,028); and gel-free sieving media (see, e.g., U.S. Pat. No. 5,624,800; Hubert and Slater, Electrophoresis, 16: 2137-2142, 1995; Mayer et al., Analytical Chemistry, 66(10):1777-1780, 1994). The electrophoresis medium may contain a nucleic acid denaturant, such as 7M formamide, for maintaining polynucleotides in single stranded form. Suitable capillary electrophoresis instrumentation are commercially available, e.g., the ABI PRISM™ Genetic Analyzer series (Applied Biosystems).

In certain embodiments, a hybridization tag complement includes a hybridization enhancer, where, as used herein, the term “hybridization enhancer” means moieties that serve to enhance, stabilize, or otherwise positively influence hybridization between two polynucleotides, e.g. intercalators (see, e.g., U.S. Pat. No. 4,835,263), minor-groove binders (see, e.g., U.S. Pat. No. 5,801,155), and cross-linking functional groups. The hybridization enhancer may be attached to any portion of a mobility modifier, so long as it is attached to the mobility modifier is such a way as to allow interaction with the hybridization tag-hybridization tag complement duplex. In certain embodiments, a hybridization enhancer comprises a minor-groove binder, e.g., netropsin, distamycin, and the like.

The skilled artisan will appreciate that an Amplicon and/or an Amplicon surrogate can also be separated based on molecular weight and length or mobility by, for example, but without limitation, gel filtration, mass spectrometry, or HPLC, and detected using appropriate methods. In certain embodiments, an Amplicon and/or an Amplicon surrogate is separated using one or more of the following forces: gravity, electrical, centrifugal, hydraulic, pneumatic, or magnetism.

In certain embodiments, an affinity tag is used to separate the element to which it is bound, e.g., an Amplicon and/or an Amplicon surrogate, from a component of a reaction composition. In certain embodiments, an affinity tag is used to bind an Amplicon and/or an Amplicon surrogate to a substrate, for example but not limited to binding a digoxygenin-labeled second amplicon to a substrate comprising anti-digoxygenin antibody. In certain embodiments, an aptamer is used to bind an Amplicon and/or an Amplicon surrogate to a substrate (see, e.g., Srisawat and Engelke, RNA 7:632-641 (2001); Holeman et al., Fold Des. 3:423-31 (1998); Srisawat et al., Nucl. Acid Res. 29(2):e4, 2001). In certain embodiments, one strand of a double-stranded Amplicon and/or Amplicon surrogate comprises an affinity tag, including without limitation, biotin, and the Amplicon and/or Amplicon surrogate is bound to a substrate comprising the corresponding affinity tag, for example but not limited to, a streptavidin-coated substrate. Thus, when the affinity tag-labeled double-stranded or partially double-stranded Amplicon or surrogate is combined with the substrate, the Amplicon or surrogate will bind to the substrate via the affinity tags. In certain embodiments, the substrate-bound double-stranded Amplicon and/or an Amplicon surrogate is denatured and the Amplicon strand that does not comprise the bound affinity tag is released from the substrate. In certain embodiments, the released strand or its surrogate is subsequently detected.

In certain embodiments, a hybridization tag, a hybridization tag complement, or a hybridization tag and a hybridization tag complement, is used to separate the element to which it is bound from an Amplicon and/or an Amplicon surrogate. In certain embodiments, hybridization tags are used to attach an Amplicon and/or an Amplicon surrogate to a substrate. In certain embodiments, a multiplicity of Amplicons and/or an Amplicon surrogates comprise the same hybridization tag. For example but not limited to, separating a multiplicity of different element:hybridization tag species using the same hybridization tag complement by tethering a multiplicity of different element:hybridization tag species to a substrate comprising the same hybridization tag complement and removing all or substantially all of the unhybridized material.

In certain embodiments, separation comprises binding an Amplicon and/or an Amplicon surrogate to a substrate, either directly or indirectly; for example but not limited to, indirectly binding an Amplicon to a glass substrate, wherein the Amplicon and/or Amplicon surrogate comprises an affinity tag such as biotin, and the substrate comprises a corresponding affinity tag, such as a streptavidin, avidin, CaptAvidin, or NeutrAvidin; or vice versa. The skilled artisan will understand that certain methods comprise at least two different separations, for example a first bulk separation and a second separation wherein, for example, an Amplicon and/or an Amplicon surrogate comprising an affinity tag is attached to a substrate comprising a corresponding affinity tag. For example, but without limitation, separating an Amplicon comprising a DNP affinity tag by capillary electrophoresis and then tethering the DNP-Amplicon indirectly to a particular address on a substrate comprising anti-DNP antibody; separating an Amplicon and/or an Amplicon surrogate comprising an hybridization tag by RP-HPLC and then hybridizing the Amplicon and/or Amplicon surrogate to a glass, mica, or silicon substrate comprising the corresponding hybridization tag complement; or binding a biotinylated double-stranded Amplicon and/or an Amplicon surrogate to a streptavidin-coated Substrate to separate it from unbound components, denaturing the double-stranded Amplicon and/or an Amplicon surrogate to release the non-biotinylated strand of the bound Amplicon and/or Amplicon surrogate, then subjecting the released single strand to a mobility dependent analytical technique, including without limitation, capillary electrophoresis or mass spectrometry.

In certain embodiments, a substrate is derivatized or coated to enhance the binding of an affinity tag, an Amplicon and/or an Amplicon surrogate, a hybridization tag complement, or combinations thereof. Exemplary substrate treatments and coatings include poly-lysine coating; aldehyde treatment; amine treatment; epoxide treatment; sulphur-based treatment (e.g., isothiocyanate, mercapto, thiol); coating with avidin, streptavidin, biotin, or derivatives thereof; and the like. Descriptions of derivatization techniques and procedures to enhance capture moiety binding can be found in, among other places, Microarray Analysis; G. MacBeath and S. Schreiber, Science 289:1760-63 (2000); A, Talapatra, R. Rouse, and G. Hardiman, Proteogenomics 3:1-10 (2002); Microarray Methods and Applications-Nuts and Bolts, G. Hardiman, ed., DNA Press (2003); B. Houseman and M. Mrksich, Trends in Biochemistry 20:279-81 (2002); S. Carmichael et al., A Simple Test Method for Covalent Binding Microarray Surfaces, NoAb BioDiscoveries Microarray Technical Note #010516SC; P. Galvin, An introduction to analysis of differential gene expression using DNA microarrays, The European Working Group on CTFR Expression (4-02-2003); and Zhu et al., Curr. Opin. Chem. Biol. 7:55-63 (2003). Pretreated substrates and derivatization reagents and kits are commercially available from several sources, including CEL Associates, Pearland Tex.; Molecular Probes, Eugene Oreg.; Quantifoil MicroTools GmbH, Jena Germany; Xenopore Corp., Hawthorne, N.J.; NoAb BioDiscoveries, Mississauga, Ontario, Canada; TeleChem International, Sunnyvale, Calif.; CLONTECH Laboratories, Inc., Palo Alto Calif.; and Accelr8 Technology Corp., Denver, Colo. In certain embodiments, the substrate-bound capture moiety comprises an amino acid, for example but not limited to, antibodies, peptide aptamers, peptides, avidin, streptavidin, biotin, and the like. In certain embodiments, the substrate-bound capture moiety comprises a nucleotide, fore example but not limited to, hybridization tag complements, nucleic acid aptamers, and chimeric oligomers further comprising PNAs, pcPNAs, LNAs, 2′ O-alkyl nucleotides, and the like.

In certain embodiments, detecting step comprises separating and/or detecting an Amplicon and/or an Amplicon surrogate using an instrument, i.e., using an automated or semi-automated detection means that can, but need not, comprise a computer algorithm. In certain embodiments, the detection step is combined with or is a continuation of a separating step, for example but not limited to a capillary electrophoresis instrument comprising a fluorescent scanner and a graphing, recording, or readout component; a capillary electrophoresis instrument coupled with a mass spectrometer; a chromatography column coupled with an absorbance monitor or fluorescence scanner and a graph recorder, or with a mass spectrometer; or a microarray with a data recording device such as a scanner or CCD camera. In certain embodiments, the detecting step is combined with the amplifying step and the quantifying and/or identifying step, for example but not limited to, real-time analysis such as Q-PCR. Exemplary means for performing a detecting step include capillary electrophoresis instruments, for example but not limited to, the ABI PRISM® 3100 Genetic Analyzer, ABI PRISM® 3100-Avant Genetic Analyzer, ABI PRISM® 3700 DNA Analyzer, ABI PRISM® 3730 DNA Analyzer, ABI PRISM® 3730x/DNA Analyzer (all from Applied Biosystems); the ABI PRISM® 7300 Real-Time PCR System; the ABI PRISM® 7700 Sequence Detection System; mass spectrometers; and microarrays and related software such as the Applied Biosystems Array System with the Applied Biosystems 1700 Chemiluminescent Microarray Analyzer and other commercially available array systems available from Affymetrix, Agilent, Illumina, and Amersham Biosciences, among others (see also Gerry et al., J. Mol. Biol. 292:251-62, 1999; De Bellis et al., Minerva Biotec 14:247-52, 2002; and Stears et al., Nat. Med. 9:140-45, including supplements, 2003). Exemplary software for reporter group detection, data collection, and analysis includes GeneMapper™ Software, GeneScan® Analysis Software, and Genotyper® software (all from Applied Biosystems).

In certain embodiments, separating or detecting comprises flow cytometry methods, including without limitation fluorescence-activated sorting (see, e.g., Vignali, J. Immunol. Methods 243:243-55, 2000). In certain embodiments, detecting comprises: separating an Amplicon and/or an Amplicon surrogate using a mobility-dependent analytical technique, such as capillary electrophoresis; monitoring the eluate using, for example but without limitation, a fluorescent scanner, to detect the Amplicons and/or Amplicon surrogates as they elute; and evaluating the fluorescent profile of the Amplicons and/or Amplicon surrogates, typically using detection and analysis software, such as an ABI PRISM® Genetic Analyzer using GeneScan® Analysis Software (both from Applied Biosystems). In certain embodiments, determining comprises a plate reader and an appropriate illumination source.

In certain embodiments, the Amplicons and/or Amplicon surrogates do not comprise reporter groups, but are detected and quantified based on their corresponding mass-to-charge ratios (m/z). In certain embodiments, a multiplicity of Amplicons and/or Amplicon surrogates, are separated by liquid chromatography or capillary electrophoresis, subjected to ESI or to MALDI, and detected by mass spectrometry. Descriptions of mass spectrometry can be found in, among other places, The Expanding Role of Mass Spectrometry in Biotechnology, Gary Siuzdak, MCC Press, 2003. Exemplary mass spectrometers for use in the current teachings include the API 2000™ LC/MS/MS System, API 3000™ LC/MS/MS System, API 4000™ LC/MS/MS System, API 4000™ QTRAP™ System, QSTAR® System, QTRAP™ System, Applied Biosystems 4700 Proteomics Analyser, and Voyager™ Biospectrometry™ series instruments (all from Applied Biosystems); Premier and Q-TOF instruments, including associated software and appropriate front-end separation system(s) (Waters); and LTQ series, LCQ series, and Quantum instruments, including associated software and appropriate front-end separation system(s) (ThermoFinnegan).

In certain embodiments, Amplicons and/or Amplicon surrogates are hybridized or attached to a substrate, including without limitation, a microarray or a bead. In certain embodiments, a substrate-bound Amplicon and/or a substrate-bound Amplicon surrogate do not comprise a reporter group, but are detected due to the hybridization of a labeled entity to the bound Amplicon and/or bound Amplicon surrogate. Such labeled entity include without limitation, a labeled hybridization tag complement, a reporter probe such as a molecular beacon, a light-up probe, a labeled LNA probe, a labeled PNA probe, or a capture probe of the substrate. In certain embodiments, the labeled entity comprises a fluorescent reporter group and quencher.

In certain embodiments, detecting comprises detecting a reporter probe, the reporter group of a released hybridization tag complement, or a part of a hybridization tag complement. For example but not limited to, hybridizing an Amplicons and/or an Amplicon surrogate to a labeled reporter probe comprising a quencher, including without limitation, a molecular beacon, including stem-loop and stem-free beacons, a TaqMan® probe, a LightSpeed™ PNA probe, or a microarray capture probe. In certain embodiments, the hybridization occurs in solution such as hybridizing a molecular beacon to an Amplicon. In other embodiments, the Amplicon, Amplicon surrogate, or reporter probe is substrate-bound and upon hybridization of the corresponding reporter probe, Amplicon, or Amplicon surrogate, fluorescence is detected (see, e.g., EviArrays™ and EviProbes™, Evident Technologies). In certain embodiments, such hybridization events are simultaneously or near-simultaneously detected and quantified.

In certain embodiments, detecting comprises a single-stranded Amplicon or Amplicon surrogate, for example but not limited to, detecting a reporter group that is integral to the single-stranded molecule being detected, such as a fluorescent reporter group that is incorporated into an Amplicon or the reporter group of a released hybridization tag complement (an exemplary Amplicon surrogate); a reporter group on a molecule that hybridizes with the single-stranded Amplicon being detected, such as a reporter probe of the current teachings, a hybridization tag complement, or conventional reporter probes such as a molecular beacon, including without limitation, PNA beacons and LNA beacons, a TaqMan®) probe, a scorpion primer, or a light-up probe.

In certain embodiments, a double-stranded Amplicon or Amplicon surrogate is detected. Typically such double-stranded Amplicons or Amplicon surrogates are detected by triplex formation or by local opening of the double-stranded molecule, using for example but without limitation, a PNA opener, a PNA clamp, and triplex forming oligonucleotides (TFOs), either reporter group-labeled or used in conjunction with a labeled entity such as a molecular beacon (see, e.g., Drewe et al., Mol. Cell. Probes 14:269-83, 2000; Zelphati et al., BioTechniques 28:304-15, 2000; Kuhn et al., J. Amer. Chem. Soc. 124:1097-1103, 2002; Knauert and Glazer, Hum. Mol. Genet. 10:2243-2251, 2001; Lohse et al., Bioconj. Chem. 8:503-09, 1997). In certain embodiments, an Amplicon and/or an Amplicon surrogate comprises a stretch of homopurine sequences.

In certain embodiments, detecting comprises measuring or quantifying the detectable signal of a reporter group or the change in a detectable signal of a reporter group, typically due to the presence of an Amplicon and/or Amplicon surrogate. For example but not limited to, an unhybridized reporter probe may emit a low level, but detectable signal that quantitatively increases when hybridized, including without limitation, certain molecular beacons, LNA probes, PNA probes, and light-up probes (see, e.g., Svanik et al., Analyt. Biochem. 281:26-35, 2000; Nikiforov and Jeong, Analyt. Biochem. 275:248-53, 1999; and Simeonov and Nikiforov, Nucl. Acids Res. 30:e91, 2002). In certain embodiments, detecting comprises measuring fluorescence polarization. Those in the art understand that the separation or detecting means employed are generally not limiting. Rather, a wide variety of separation and detecting means are within the scope of the disclosed methods and kits.

It is to be appreciated that, according to the present teachings, a step for generating an Amplicon can be performed using an appropriate amplifying means and/or technique, for example but not limited to, the amplification techniques disclosed herein; a step for detecting, a step for identifying a polynucleotide target, a step for quantitating a polynucleotide target, or combinations thereof, can be preformed using appropriate techniques, including without limitation, an appropriate instrument, for example but not limited to, those techniques and exemplary instruments disclosed herein. In some embodiments, a step for generating a first product can be performed using, among other things, the disclosed reverse primers of the first primer sets and an extending enzyme; a step for generating a first amplicon can be performed using, among other things, the disclosed forward primers of the first primer sets and an extending enzyme; a step for generating an additional first amplicon can be performed using, among other things, the disclosed first primer sets and an extending enzyme; a step for generating a second amplicon can be performed using, among other things, the second primer sets and an extending enzyme; and a step for detecting the second amplicons or their surrogates can be performed using, among other things, the disclosed detecting means, which may or may not include the disclosed separating means; and a step for identifying or for quantitating a polynucleotide target can be performed using, among other things, the disclosed substrates, instruments, software, or combinations thereof.

### IV. Certain Exemplary Methods

Certain of the disclosed methods are directed to quantitating known polynucleotides of interest, particularly but not limited to, small RNA molecules such as miRNA, siRNA, stRNA, and other ncRNA. In such methods, the sequence of the target polynucleotide is known and first primer sets and reporter probes can be designed based on the known sequence. Second primer sets can be designed to serve as: (i) amplification primers for individual first amplicons and additional first amplicons and may or may not encode target-specific hybridization tags, useful for subsequent isolation and/or identification, (ii) universal primers, for example but not limited to, multiplexed amplification of a multiplicity of first amplicons and/or additional first amplicons, typically in a uniform manner, or (iii) a combination of a universal primer and a target-specific primer that encodes a target-specific hybridization tag.

Other disclosed methods are directed to identifying unknown target polynucleotides, particularly but not limited to, small RNA molecules such as miRNA, siRNA, stRNA, and other ncRNA. The sequence of interest is not known, although partially sequence information may be known or predicted. For illustration purposes but not as a limitation, several miRNA predictive algorithms are available (see, e.g., MiRscan, available on the web at genes/mit.edu/mirscan; miRseeker; and Carter et al., Nucl. Acids Res. 29(19):3928-38, 2001). The scientific literature and available databases (see, e.g., the miRNA Registry, on the world-wide web at sanger-ac.uk/Software/Rfam/miRNA/index) can be analyzed to identify possible regions of homology, at one or both ends of potential miRNA targets that can be further evaluated using routine experimentation. Bioinformatics searching of the gDNA for possible stem-loop structures can also indicate potential miRNA targets for evaluation according to the current teachings. Additionally, unknown sequences can be identified empirically using the disclosed methods and compositions. In some embodiments, one or both primers of a fist primer set for identifying a polynucleotide target, including without limitation, a small RNA molecule, comprise a target-binding portion including 6, 7, 8, 9, or 10 random or degenerate nucleotides, including without limitation, a universal base.

While the certain embodiments of these methods employ “RT-PCR-PCR like” amplification techniques, other amplification techniques are also contemplated. Further, certain embodiments of the disclosed methods comprise a single reaction composition in which Amplicons are generated. Other embodiments comprise two or more reaction compositions, including without limitation, a multiplex format comprising a first reaction composition in which first products, first amplicons and additional first amplicons are generated, and a multiplicity of different second reaction compositions in which second amplicons are generated.

An overview of some aspects of certain disclosed methods is depicted in FIGS. 1A and 1B for illustration purposes, but is not intended to limit the current teachings in any way. As shown at the top of FIG. 1A, an exemplary miRNA target hybridizes to a corresponding reverse primer of a first primer set and in the presence of an extending enzyme, the hybridized reverse primer is extended and a first product is formed. Under appropriate reaction conditions, the forward primer hybridizes with the first product and another reverse primer hybridizes to the target. Those in the art will appreciate that according to conventional methodology, the first product-target duplex is denatured before the forward and reverse primers can bind, often in a thermocycler. Surprisingly, the inventors have observed that when the target is an miRNA, both the forward and reverse primers can be incorporated isothermally, i.e., without a denaturation step. Without being limited to a particular theoretical basis, this may be due to the concentration of the miRNA-first target duplex (typically in the 10⁻¹⁵ (fM) to 10⁻¹² (pM) range) relative to the concentration of the first primer set (typically in the 10⁻⁸ (nM) to 10⁻⁶ (μM) range). Under these conditions, the forward primer might displace 5′-end of the miRNA target from the target-first product duplex and be extended by an extending enzyme, even at sub-optimum temperatures for enzyme activity. For example, in certain embodiments wherein the target is a small RNA molecule, the first reaction composition is incubated at about 20° C. for several minutes (for example, but not limited to 10-30 minutes) and then the temperature is raised to optimize or at least enhance the activity of the extending enzyme (typically a reverse transcriptase in such an embodiment). Thus, in certain embodiments, a denaturation step is included prior to the step of generating first amplicons, while in other embodiments, it is optional. The temperature of the reaction composition is raised to inactivate the reverse transcriptase (if any) and/or to activate a second extending enzyme, if appropriate (for example, a “hot start” polymerase). The reaction composition is then cycled between denaturation temperatures and annealing/extension temperatures (for example but not limited to, 95° C. or above for 10-20 second, then about 60° C. for approximately 1 minute) for a limited number of cycles (typically 12, 11, 10, 9, 8, 7, 6, 5, or 4 cycles) to generate first amplicons and additional first amplicons.

Returning to FIG. 1, in certain embodiments, after the first amplicons and the additional first amplicons are generated, a second primer set and optionally, an extending enzyme are added (see FIG. 1B top) to form a second reaction composition. In other embodiments, discussed below, the second primer set(s) are included in the first reaction composition. The reaction composition is heated to a temperature sufficient to denature the first amplicons and the additional first amplicons. The reaction composition is cooled to allow the primers of the second primer set to hybridize to the separated strands of the first amplicons or the additional first amplicons and the hybridize primers of the second primer set are extending by the extending enzyme to generate second amplicons and the cycle is repeated as necessary, as shown in the top half of FIG. 1B.

In certain embodiments, a reporter probe is added to the second reaction composition when the second primer set and optional extending enzyme are added. In other embodiments, reporter probes are added at a later step. Those in the art will appreciate that when detection comprises using reporter probes in a nuclease assay including but not limited to a TaqMan® assay, or a probe extension assay, such as with scorpion primers, an appropriate DNA polymerase (which may or may not be the same as the second extending enzyme) needs to be included in the reaction composition (shown as DNA polymerase* in FIG. 1B). The reaction is cycled, depending on the reporter probes and the nature of the detection assay employed, and the reporter probes or their surrogates (for example but not limited to cleaved reporter groups) are detected and the corresponding target is identified or quantitated, as shown at the bottom of FIG. 1B.

Those in the art will appreciate that detection can comprise a variety of reporter probes with different mechanisms of action and that detection can be performed either in real-time or at an end-point. It will also be appreciated that detection can comprise reporter groups that are incorporated into the Amplicons, either as part of labeled primers or due to the incorporation of labeled dNTPs during an amplification, or attached to Amplicons, for example but not limited to, via hybridization tag complements comprising reporter groups or via linker arms that are integral or attached to Amplicons. Detection of unlabeled Amplicons, for example using mass spectrometry is also within the scope of the current teachings.

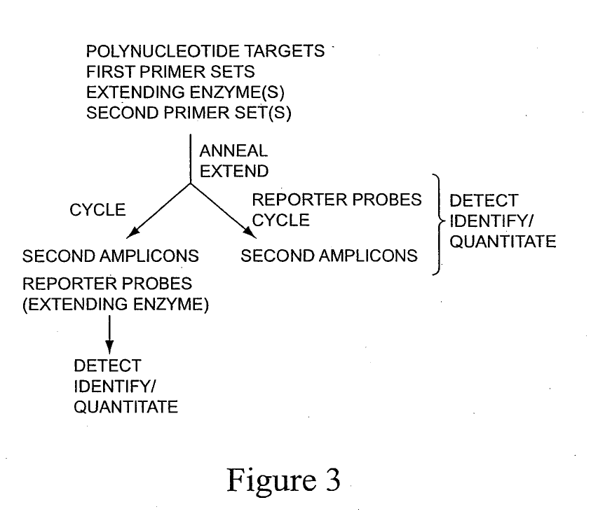

In certain embodiments of the disclosed methods, a single reaction composition is formed and two, three or four amplification steps (depending on the reaction format) occur in the same reaction composition and typically, the same reaction vessel (see, e.g., FIG. 3). According to certain embodiments of the disclosed methods, a first reaction composition comprises a polynucleotide target, a first primer set, and an extending enzyme; and a first product, a first amplicon, an additional first amplicon, or combinations thereof, are generated and detected; and the target polynucleotide is identified and/or quantitated.

In certain embodiments, the single reaction composition further comprises a second primer set. The first and second primers of the second primer set are used to amplify the first amplicon and/or additional first amplicon to generate a second amplicon. In certain embodiments, a primer of the second primer set is a universal primer. In certain embodiments, both primers of at the second primer set comprise universal primers. In certain embodiments, one of the second primers is a universal primer and the corresponding primer comprises a hybridization tag that typically encodes a target-specific sequence that can be subsequently used to correlate the second amplicon to its corresponding polynucleotide target. In certain embodiments, a primer of the second primer set comprises an affinity tag. In certain embodiments, the second amplicon is cycled with additional primers of the second primer set to generate more second amplicons. In certain embodiments, the second amplicons or their surrogates are detected and the corresponding polynucleotide target is identified and/or quantitated.

In certain embodiments, a polynucleotide target comprises a small RNA molecule, the extending enzyme comprises a reverse transcriptase or a DNA polymerase with reverse transcriptase activity, and the first product comprises a reverse-transcribed product. In certain embodiments, at least two different extending enzymes are used, including a reverse transcriptase and a DNA polymerase.

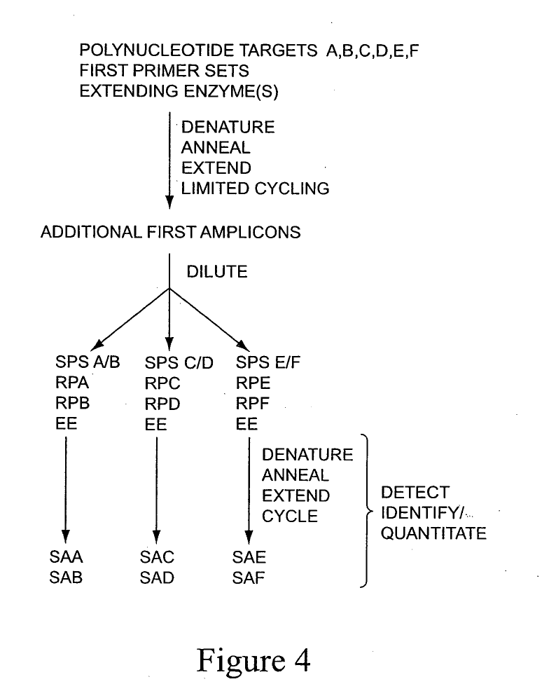

In certain embodiments, the disclosed methods comprise forming at least two different reaction compositions (see, e.g., FIG. 4). In essence, two primer sets per polynucleotide target are used in three or four amplification steps that occur in two different reaction compositions and can, but need not, take place in the same reaction vessel. The amplification steps that typically occur in the first reaction composition include: (i) generating a first product using the reverse primer of the first primer set, (ii) generating a first amplicon using the first product as the template and the corresponding forward primer of the first primer set, and optionally, (iii) generating additional first amplicons using forward and reverse primers of the corresponding first primer set. When the first stage is completed, the resulting reacted first reaction composition is combined with the corresponding first and second primers of the second primer set(s), which may, but need not include universal primers, primers comprising unique hybridization tags, or both, and (iv) second amplicons are generated using the first amplicons, and where appropriate, the additional first amplicons, as templates. In certain embodiments, the first stage reactions are performed in a multiplex first reaction composition. In certain embodiments, the second stage reaction is performed in multiplex, which may but need not include a multiplicity of parallel lower-plexy second reaction compositions. The second stage reaction can, but need not, include real-time detection.

In certain embodiments, a first reaction composition is formed comprising a polynucleotide target, a first extending enzyme, a second extending enzyme, and a first primer set, comprising a forward primer and a reverse primer. In certain embodiments, the first extending enzyme and the second extending enzyme are: (i) the same, for example but not limited to, a thermostable DNA polymerase that, under certain conditions possesses reverse transcriptase activity, such as Tth polymerase or a reverse transcriptase that, under certain conditions possesses DNA polymerase activity, such as AMV reverse transcriptase; or (ii) different, for example but not limited to, a first extending enzyme comprising a retrovirus reverse transcriptase, such as AMV reverse transcriptase (under conditions where only reverse transcription occurs) and a second extending enzyme, such as Thermus aquaticus (Taq) polymerase. Under suitable conditions, a first reaction product and a first amplicon are generated in the first reaction composition. In certain embodiments, an additional first amplicon is also generated in the first reaction composition using the first primer set.

A second reaction composition is formed comprising: (i) the first amplicon, the additional first amplicon, or the first amplicon and the additional first amplicon of the first reaction composition, (ii) a second primer set, and typically, (iii) a third extending enzyme. In certain embodiments, the first amplicon or the first amplicon and the additional first amplicon is diluted prior to or during the formation of the second reaction composition. Under suitable reaction conditions, the first amplicon, the additional first amplicon, or both, are amplified using the primers of the second primer set and the third extending enzyme and a second amplicon is generated. In certain embodiments, a second reaction composition further comprises a reporter probe, an intercalating agent, or both. In certain embodiments, a second amplicon comprises a hybridization tag, a mobility modifier, an affinity tag, a reporter group, or combinations thereof. The second amplicon or its surrogate is detected and the corresponding target polynucleotide is identified and/or quantitated.

In certain embodiments, a multiplicity of different second reaction compositions are formed. In certain embodiments, a diluted or undiluted reacted first reaction composition is placed into one or more different wells of a multi-well reaction vessel, including without limitation a multi-well plate or a multi-chambered microfluidic device such as a TaqMan® Low Density Array (Applied Biosystems, Foster City, Calif.). In certain embodiments, at least two of the different reaction wells, including without limitation, at least two different reaction chambers, comprise: (i) an extending enzyme, (ii) a second primer set, and (iii) a reporter probe, wherein a reporter probe in one well or chamber is different from a reporter probe in another well or chamber. In certain embodiments, only a subset of the total number of different polynucleotide target being evaluated are detected, identified, and/or quantitated in a single reaction well or reaction chamber.

In certain embodiments, the target polynucleotide comprises a small RNA molecule, an extending enzyme comprises a reverse transcriptase or a DNA polymerase with reverse transcriptase activity, and the first product comprises a reverse-transcribed product.

Certain embodiments of the disclosed methods comprise at least three reaction compositions. A first product is generated in a first reaction composition comprising a reverse primer of a first primer set, a polynucleotide target, and a first extending enzyme. A first amplicon and an additional first amplicon is generated in a second reaction composition that comprises the reacted first reaction composition or at least part of the reacted first reaction composition, a forward primer of the corresponding first primer set, and optionally, a second extending enzyme. Typically, the second reaction composition is thermocycled a limited number of times, for example 12, 10, 9, 8, 7, 6, 5, 4, or 3 cycles. Second amplicons are generated in a third reaction composition that comprises the reacted second reaction composition or at least part of the reacted second reaction composition, a second primer set, and optionally, a third extending enzyme. The second amplicons or their surrogates are detected and the corresponding target polynucleotides are identified and/or quantitated.

The reacted first reaction composition, the reacted second reaction composition, or the reacted first reaction composition and the reacted second reaction composition can, but need not be, diluted prior to or during the forming of the second or third reaction compositions, respectively. The first extending enzyme, the second extending enzyme, and the third extending enzyme can be the same or different; and the second and third extending enzymes can be the same while the first extending enzyme is different. In certain embodiments, a multiplex reaction occurs in the first reaction composition, the second reaction composition, the third reaction composition, or combinations thereof.

In certain embodiments of the disclosed methods, a multiplicity of different target polynucleotides are identified or quantitated and the third reaction composition comprises a multiplicity of different third reaction compositions, wherein a subset of the multiplicity of different target polynucleotides is analyzed in a different third reaction composition. In certain embodiments, a diluted or undiluted reacted second reaction composition is placed into one or more different wells of a multi-well reaction vessel, including without limitation a multi-well plate or a multi-chambered microfluidic device such as a TaqMan® Low Density Array (Applied Biosystems). In certain embodiments, at least two of the different reaction wells, including without limitation, at least two different reaction chambers, comprise: (i) an extending enzyme, (ii) a second primer set, and (iii) a reporter probe, wherein a reporter probe in one well or chamber is different from a reporter probe in another well or chamber. In certain embodiments, only a subset of the total number of different polynucleotide target being evaluated are detected, identified, and/or quantitated in a single reaction well or reaction chamber.

### V. Certain Kits

The instant teachings also provide kits designed to facilitate the subject methods. Kits serve to expedite the performance of the disclosed methods by assembling two or more components required for carrying out certain methods. Kits can contain components in pre-measured unit amounts to minimize the need for measurements by end-users and can also include instructions for performing one or more of the disclosed methods. Typically, kit components are optimized to operate in conjunction with one another.

The disclosed kits may be used to identify, detect, and/or quantitate target polynucleotides, including small RNA molecules and polynucleotides comprising deoxyribonucleotides. In certain embodiments, kits comprising a forward primer comprising a target-binding portion containing six, seven eight, nine or ten nucleotides, or a reverse primer comprising a target-binding portion containing six, seven, eight, nine, or ten nucleotides are disclosed. In certain embodiments, such kits comprise a first primer set that includes a forward and a corresponding reverse primer. In certain embodiments, the disclosed kits further comprise, a second primer set, including without limitation a universal forward primer, a universal reverse primer, or both; a reporter probe; a reporter group; a reaction vessel, including without limitation, a multi-well plate or a microfluidic device; a substrate; a buffer or buffer salt; a surfactant; or combinations thereof. In certain embodiments, the disclosed kits may further comprise a first extending enzyme, a second extending enzyme, and/or a third extending enzyme.

### IV. Exemplary Embodiments

The current teachings, having been described above, may be better understood by reference to examples. The following examples are intended for illustration purposes only, and should not be construed as limiting the scope of the disclosed teachings in any way.

### EXAMPLE 1

### Detection and Quantitation of a Small RNA Molecule Using Various Reporter Probe Constructs

A polynucleotide target comprising the sequence: gaagagauacgcccugguuccu (SEQ ID NO:1) was synthesized using conventional methodology. A corresponding first primer set was also synthesized, comprising (i) an forward primer with the sequence: [ACCGACTCCAGCTCCCGAAC]GAAGAGAT (SEQ ID NO:2) that includes a target-binding portion of eight nucleotides that are the same as the 5′-end of the synthetic small RNA molecule target (shown underlined) and upstream, a primer-binding portion (shown in brackets) for a first universal primer, and (ii) a reverse primer with the sequence [GTGTCGTGGAGTCGGCAA]AGGAACCA (SEQ ID NO:3) that includes a target-binding portion of eight nucleotides that are complementary to the 3′-end of the synthetic small RNA molecule target (shown underlined) and upstream, a second primer-binding portion (shown in brackets) for a second universal primer. Four different reporter probes, shown in Table 1, were also prepared, including: (i) a probe (“DNA” in Table 1) comprising deoxyribonucleotides, the fluorescent reporter group 6-carboxyfluorescein (“FAM”) and a minor groove binder (“MGB”); (ii) a chimeric probe (“2DNA-LNA” in Table 1) comprising two deoxyribonucleotides (shown underlined), ten LNAs (shown in parentheses), FAM, and a minor groove binder (MGB); (iii) a chimeric probe (“2DNA-OMC” in Table 1) comprising two deoxyribonucleotides (shown underlined), eighteen 2′-O-methyinucleotides (shown in parentheses), FAM, and an MGB; and (iv) a PNA probe (“PNA” in Table 1) comprising thirteen PNAs, a FAM fluorescent reporter group, and the quenching reporter group 4-(4′-dimethylaminophenylazo)benzoic acid (“Dabcyl” in Table 1).

TABLE 1
Reporter Probes

| | |
|---|---|
| Probe | Composition |

| | | |
|---|---|---|
| DNA | FAM-AAGAGATACGCCCTGGTTCCT- | (SEQ ID NO:4) |
| | MGB | |
| 2DNA-LNA | FAM-AG(ATACGCCCTG)-MGB | |
| 2DNA-OMC | FAM-AA(GAGATACGCCCTGGTTCC)- | |
| | MGB | |
| PNA | FAM-AGATACGCCCTGG-Dabcyl | |

A multiplicity of first reaction compositions were formed comprising 1 microliter (μL) serial ten-fold dilutions of the target in ddH₂O (100 femptomole (fM), 10 fM, 1 fM, 10 attamole (aM), or 1 aM), 1 uL of the first primer set (1 μM forward and reverse primers), 5 μL 2× RT-PCR Master Mix (comprising AmpliTaq Gold® DNA polymerase; Applied Biosystems), 3.75 μL ddH₂O, and 0.25 μL MultiScribe™ Reverse Transcriptase with 40× RNase inhibitor (Applied Biosystems), in final volumes of 10 μL. To generate the reverse-transcribed products, the first amplicons, and the additional first amplicons, these first reaction compositions were heated at 37° C. for 30 minutes, then 95° C. for 10 minutes, cycled ten times between 95° C. for 15 seconds and 60° C. for one minute, then cooled to 4° C. Each of the cycled first reaction compositions comprising reverse-transcribed product, first amplicons, and additional first amplicons was diluted 100-fold in ddH₂O.

A corresponding multiplicity of second reaction compositions were formed, each comprising 1 μL of the appropriate diluted reacted first reaction composition, 2.5 μL of the second primer set comprising the universal primers GTGTCGTGGAGTCGGCAA (SEQ ID NO:5) and ACCGACTCCAGCTCCCGAAC (SEQ ID NO:6)(10 μM of the first and second universal primers), 1 μL of the appropriate reporter probe (5 μM of either the DNA, 2DNA-LNA, or 2DNA-OMC; all shown in Table 1) 12.5 μL 2× TaqMan® Universal Master Mix No AmpErase® UNG (part no. 4324018, Applied Biosystems), and 9 μL ddH₂O, in final volumes of 25 μL. To generate second amplicons these second reaction compositions were transferred to an ABI 7700 Sequence Detection System (Applied Biosystems) where they were heated to 95° C. for 10 minutes, cycled 40 times between 95° C. for 15 seconds and 60° C. for one minute, then cooled to 4° C. The threshold cycle value (Cₜ) for each reaction was determined using the, as shown in Table 2.

TABLE 2
Cₜ Values for DNA, 2DNA-LNA, and 2DNA-OMC Probes.

| Target | | | |
|---|---|---|---|
| Concentration | DNA Probe | 2DNA-LNA | 2DNA-OMC |
| 100 fM | 19.43 | 20.85 | 19.87 |
| 10 fM | 22.37 | 23.86 | 22.24 |
| 1 fM | 27.19 | 29.05 | 26.01 |
| 100 aM | 29.3 | 40 | 40 |
| 10 aM | 31.43 | 40 | 40 |
| 1 aM | 37.57 | 40 | 40 |

### EXAMPLE 2

### Comparison of Cleavable DNA Reporter Probe with PNA Beacon Reporter Probe

To evaluate the PNA reporter probe (shown in Table 1), a similar reaction protocol was performed except that the second reaction composition was transferred to an ABI PRISM 7900HT Sequence Detection System (Applied Biosystems) cycled forty times between 95° C. for 15 seconds, 50° C. for 1 minute, and 60° C. for 1 minute, and Cₜ values obtained, as shown in Table 3.

TABLE 3
Ct Values for DNA and PNA Probes

| Target | | |
|---|---|---|
| Concentration | DNA Probe | PNA Probe |
| 100 fM | 16.31 | 20.11 |
| 10 fM | 19.37 | 23.27 |
| 1 fM | 23.36 | 27.22 |
| 100 aM | 25.84 | 30.29 |
| 10 aM | 27.01 | 33.45 |
| 1 aM | 31.00 | 40 |

### EXAMPLE 3

### Effect of “Background RNA” in First Reaction Composition

To evaluate the effect of total RNA concentration of the detection and quantitation of a target, an exemplary assay was performed in parallel triplicate sets of first reaction compositions comprising 100 fM, 10 fM, 1 fM, 100 aM, 10 aM, or 1 aM of the target, each in the presence and absence of “background RNA” (12 different reaction compositions, each in triplicate). Target quantitation was performed essentially as described in Example 1, except that to six triplicate sets of first reaction compositions 100 ng Universal Human Reference total RNA (“100 ng UHR” in Table 4; UHR TotalRNA, 1 mg/mL, Applied Biosystems Part No. 4345048 diluted into ddH₂O) was added and to the other six triplicate sets, buffer without UHR was added (“0 ng UHR” in Table 4). The second reaction compositions comprised the DNA reporter probe of Example 1, but not any other reporter probes. The reaction compositions were cycled and Ct values determined using the ABI PRISM® 7700 Sequence Detection System. The mean Ct values for all twelve triplicate sets and the standard deviation for each is shown in Table 4.

TABLE 4
Effect of “background RNA” on Detection and Quantitation.

| Target | Mean Ct | Std. Dev. | Mean Ct | Std. Dev. |
|---|---|---|---|---|
| Concentration | 0 ng UHR | 0 ng UHR | 100 ng UHR | 100 ng UHR |
| 100 Fm | 21.57 | 0.07 | 21.83 | 0.23 |
| 10 fM | 24.19 | 0.42 | 24.86 | 0.16 |
| 1 fM | 29.25 | 0.06 | 27.14 | 0.50 |
| 100 aM | 31.24 | 0.27 | 36.48 | 1.63 |
| 10 aM | 33.66 | 0.44 | 39.73 | 0.46 |
| 1 aM | 36.43 | 2.18 | 40.00 | 0.00 |

### EXAMPLE 4

### Evaluation of Target-Binding Portion Size

To evaluate the effect of the size of the polynucleotide-binding portions of the forward and reverse primers on the efficiency of detection, a series of primers comprising target-binding portions containing six, seven, eight, nine, or ten nucleotides were synthesized. The forward and reverse primers of five different first primer sets are shown in Table 5, with the target-binding portions shown underlined and the primer-binding portions shown in brackets.

TABLE 5
First Primer Sets with varying size target-binding portions.

| Primer | | |
|---|---|---|
| Set | Forward primer sequence | Reverse primer sequence |
| 10-mer | [ACCGACTCCAGCTCCCGAAC]GAAGAGATAC | [GTGTCGTGGAGTCGGCAA]AGGAACCAGG |
| | (SEQ ID NO:7) | (SEQ ID NO:8) |
| 9-mer | [ACCGACTCCAGCTCCCGAAC]GAAGAGATA | [GTGTCGTGGAGTCGGCAA]AGGAACCAG |
| | (SEQ ID NO:9) | (SEQ ID NO:10) |
| 8-mer | [ACCGACTCCAGCTCCCGAAC]GAAGAGAT | [GTGTCGTGGAGTCGGCAA]AGGAACCA |
| | (SEQ ID NO:2) | (SEQ ID NO:3) |
| 7-mer | [ACCGACTCCAGCTCCCGAAC]GAAGAGA | [GTGTCGTGGAGTCGGCAA]AGGAACC |
| | (SEQ ID NO:11) | (SEQ ID NO:12) |
| 6-mer | [ACCGACTCCAGCTCCCGAAC]GAAGAG | [GTGTCGTGGAGTCGGCAA]AGGAAC |
| | (SEQ ID NO:13) | (SEQ ID NO:14) |

A series of first reaction compositions were prepared as described in Example 1, with the 10-mer first primer set being combined with each of 100 fM, 10 fM, 1 fM, 100 aM, 10 aM, and 1 aM synthetic RNA target; the 9-mer first primer set being combined with each of 100 fM,10 fM, 1 fM, 100 aM,10 aM, and 1 aM synthetic RNA target; and so forth. The reporter probe in the second reaction composition was the DNA probe (SEQ ID NO:4). The method was otherwise performed as described in Example 1 and Ct values determined for each first primer set-target concentration combination, as shown in Table 6.

TABLE 6
Effect of target-binding portion length on
detection efficiency (C_{T} values).

| Target | both 10- | both 9- | both 8- | both 7- | both 6- |
|---|---|---|---|---|---|
| concentration | mers | mers | mers | mers | mers |
| 100 fM | 20.35 | 21.03 | 23.15 | 25.56 | 28.76 |
| 10 fM | 20.91 | 24.25 | 26.33 | 28.53 | 31.78 |
| 1 fM | 20.55 | 26.5 | 28.2 | 31.2 | 32.37 |
| 100 aM | 21 | 26.33 | 29.69 | 40 | 40 |
| 10 aM | 20.29 | 30.32 | 31.32 | 33.92 | 40 |
| 1 aM | 18.93 | 30.65 | 31.45 | 35.14 | 40 |

### EXAMPLE 5

### Evaluation of First Primer Set Concentration

To evaluate the effect of various concentrations of the first primer set on the detection and quantitation of small RNA molecules a series of first reaction compositions were prepared containing serial two-fold dilutions of the 8-mer first primer set (0-100 nM), the DNA reporter probe, and either 100 fM or 10 fM of the synthetic RNA target. The remainder of the method was performed and the Ct values (shown in Table 7) were obtained as described in Example 1.

TABLE 7
Effect of Primer Concentration on Detection and Quantitation.

| First Primer | Ct, 100 fM | Ct, 10 fM |
|---|---|---|
| Concentration | RNA target | RNA target |
| 100 nM | 19.56 | 23.79 |
| 50 nM | 20.53 | 23.23 |
| 25 nM | 21.16 | 25.57 |
| 12.5 nM | 22.01 | 26.44 |
| 0 nM | 40 | 40 |

### EXAMPLE 6

### Exemplary Multiplex Quantitation of miRNA Targets

To quantify a multiplicity of different target nucleotides in an exemplary multiplex quantitation assay, the 33 synthetic miRNA targets and corresponding first probe sets shown in Table 8 are synthesized or can be obtained from commercial vendors, including without limitation, Applied Biosystems. Each of the forward primers (“UF-FP”) include the same universal primer-binding portion (shown in brackets) located 5′ of the target-binding portions (shown underlined). Each of the reverse primers (“zip-RP”) include a different primer-binding portion (shown in parentheses) 5′ of the target-binding portions (shown underlined).

TABLE 8
Synthetic miRNA Targets and First Primer Sets.

| | | |
|---|---|---|
| miRNA | | |
| target | target sequence | First Primer Sets: (UF-FP/ zip-RP) |

| | | | |
|---|---|---|---|
| let-7a1 | ugagguaguagguuguauaguu | [ACCGACTCCAGCTCCCGAAC]AATGAGGTAG | (SEQ ID NO:16) |
| | (SEQ ID NO:15) | (TTGGCCTGTTCCCGCTCGCT)AAAACTATAC | (SEQ ID NO:17) |
| lin-4 | ucccugagaccucaaguguga | [ACCGACTCCAGCTCCCGAAC]AATCCCTGAG | (SEQ ID NO:19) |
| | (SEQ ID NO:18) | (TCGCGGACGGTGAGCACTGC)AATCACACTT | (SEQ ID NO:20) |
| mir-20 | uaaagugcuuauagugcaggua | [ACCGACTCCAGCTCCCGAAC]AATAAAGTGC | (SEQ ID NO:22) |
| | (SEQ ID NO:21) | (TTCCCGCTGGTGCCACTCCG)AATACCTGCA | (SEQ ID NO:23) |
| mir-30a | cuuucagucggauguuugcagc | [ACCGACTCCAGCTCCCGAAC]AACTTTCAGT | (SEQ ID NO:25) |
| | (SEQ ID NO:24) | (CGCTCGCTTTGGCCTGCTGC)AAGCTGCAAA | (SEQ ID NO:26) |
| mir-7 | uggaagacuagugauuuuguu | [ACCGACTCCAGCTCCCGAAC]AATGGAAGAC | (SEQ ID NO:28) |
| | (SEQ ID NO:27) | (CGCTGCGTGAGGGCTCGGAC)AAAACAAAAT | (SEQ ID NO:29) |
| mir-107 | agcagcauuguacagggcuauca | [ACCGACTCCAGCTCCCGAACA]AAGCAGCAT | (SEQ ID NO:31) |
| | (SEQ ID NO:30) | (TTCCACCCGCGTCGTCCTGC)AATGATAGCC | (SEQ ID NO:32) |
| miR159a | uuuggauugaagggagcucua | [ACCGACTCCAGCTCCCGAAC]AATTTGGATT | (SEQ ID NO:34) |
| | (SEQ ID NO:33) | (AGCACGCTTCCGGGACCCAC)AATAGAGCTC | (SEQ ID NO:35) |
| mir161 | uugaaagugacuacaucgggg | [ACCGACTCCAGCTCCCGAAC]AATTGAAAGT | (SEQ ID NO:37) |
| | (SEQ ID NO:36) | (AGGCGGTGTTGGCTCGAGGC)AACCCCGATG | (SEQ ID NO:38) |
| mir-124 | uaaggcacgcggugaaugccaag | [ACCGACTCCAGCTCCCGAAC]AATAAGGCAC | (SEQ ID NO:40) |
| | (SEQ ID NO:39) | (GAGGTTCCCGCTTCGCCCAC)AACTTGGCAT | (SEQ ID NO:41) |
| mir-210 | uugugcgugugacagcggcua | [ACCGACTCCAGCTCCCGAAC]AATTGTGCGT | (SEQ ID NO:43) |
| | (SEQ ID NO:42) | (GGTGCCACGTGATCGCTCCG)AATAGCCGCT | (SEQ ID NO:44) |
| mir-2 | uaucacagccagcuuugaugugc | [ACCGACTCCAGCTCCCGAAC]AATATCACAG | (SEQ ID NO:46) |
| | (SEQ ID NO:45) | (CTGCAGGCGCGTGAGGGAGG)AAGCACATCA | (SEQ ID NO:47) |
| mir-16 | uagcagcacguaaauauuggcg | [ACCGACTCCAGCTCCCGAAC]AATAGCAGCA | (SEQ ID NO:49) |
| | (SEQ ID NO:48) | (GCCATCCGCGCTCAGTCCAC)AACGCCAATA | (SEQ ID NO:50) |
| mir-21 | uagcuuaucagacugauguuga | [ACCGACTCCAGCTCCCGAAC]AATAGCTTAT | (SEQ ID NO:52) |
| | (SEQ ID NO:51) | (GGTGGGACGCGTCTGCCTCG)AATCAACATC | (SEQ ID NO:53) |
| mir-22 | aagcugccaguugaagaacugu | [ACCGACTCCAGCTCCCGAAC]AAAAGCTGCC | (SEQ ID NO:55) |
| | (SEQ ID NO:54) | (TCGCTTGGGTCTTCGCAGGC)AAACAGTTCT | (SEQ ID NO:56) |
| mir-26a | uucaaguaauccaggauaggcu | [ACCGACTCCAGCTCCCGAAC]AATTCAAGTA | (SEQ ID NO:58) |
| | (SEQ ID NO:57) | (TTGGGACCCTCGGGACTCGC)AAAGCCTATC | (SEQ ID NO:59) |
| mir-29 | cuagcaccaucugaaaucgguu | [ACCGACTCCAGCTCCCGAAC]AACTAGCACC | (SEQ ID NO:61) |
| | (SEQ ID NO:60) | (TCCGGACCAGCACTGCAGGC)AAAACCGATT | (SEQ ID NO:62) |
| mir-34 | uggcagugucuuagcugguugu | [ACCGACTCCAGCTCCCGAAC]AATGGCAGTG | (SEQ ID NO:64) |
| | (SEQ ID NO:63) | (TCGCGAGGGTCTTCCGGAGG)AAACAACCAG | (SEQ ID NO:65) |
| mir-181a | aacauucaacgcugucggugagu | [ACCGACTCCAGCTCCCGAAC]AAAACATTCA | (SEQ ID NO:67) |
| | (SEQ ID NO:66) | (GGTCCACCCTGCTGCTCGC)AAACTCACCG | (SEQ ID NO:68) |
| mir-200b | cucuaauacugccugguaaugaug | [ACCGACTCCAGCTCCCGAAC]AACTCTAATA | (SEQ ID NO:70) |
| | (SEQ ID NO:69) | (TCGCGACCGTCTGCTCAGGC)AACATCATTA | (SEA ID NO:71) |
| mir-223 | ugucaguuugucaaauacccc | [ACCGACTCCAGCTCCCGAAC]AATGTCAGTT | (SEQ ID NO:73) |
| | (SEQ ID NO:72) | (CGCTGGACCGCTAGGCACCC)AAGGGGTATT | (SEQ ID NO:74) |
| mir-224 | caagucacuagugguuccguuua | [ACCGACTCCAGCTCCCGAAC]AACAAGTCAC | (SEQ ID NO:76) |
| | (SEQ ID NO:75) | (TTGGGCGTTGGTGCGTCCTG)AATAAACGGA | (SEQ ID NO:77) |
| mir-323 | gcacauuacacggucgaccucu | [ACCGACTCCAGCTCCCGAAC]AAGCACATTA | (SEQ ID NO:79) |
| | (SEQ ID NO:78) | (GCTCCAGTTCCGGGTGCGCT)AAAGAGGTCG | (SEQ ID NO:80) |
| mir-324- | cgcauccccuagggcauuggugu | [ACCGACTCCAGCTCCCGAAC]AACGCATCCC | (SEQ ID NO:82) |
| 5 | (SEQ ID NO:81) | (GCGTGAGGTTGGGAGGGCGT)AAACACCAAT | (SEQ ID NO:83) |
| mir-328 | cuggcccucucugcccuuccgu | [ACCGACTCCAGCTCCCGAAC]AACTGGCCCT | (SEQ ID NO:85) |
| | (SEQ ID NO:84) | (GGTTGGGAGGCTGCGGTG)AAACGGAAGG | SEQ ID NO:86 |
| mir-10a | uacccuguagauccgaauuugug | [ACCGACTCCAGCTCCCGAAC]AATACCCTGT | (SEQ ID NO:88) |
| | (SEQ ID NO:87) | (CGTCGCTCTGGTTCGCTCGC)AACACAAATT | (SEQ ID NO:89) |
| mir-10b | uacccuguagaaccgaauuugu | [ACCGACTCCAGCTCCCGAAC]AATACCCTGT | (SEQ ID NO:91) |
| | (SEQ ID NO:90) | (GGTGACCCGTGACGCTGCCA)AAACAAATTC | (SEQ ID NO:92) |
| mir-23 | aucacauugccagggauuucc | [ACCGACTCCAGCTCCCGAAC]AAATCACATT | (SEQ ID NO:94) |
| | (SEQ ID NO:93) | (AGGCTTGGCGCTCGTCACCC)AAGGAAATCC | (SEQ ID NO:95) |
| mir-27 | uucacaguggcuaaguuccgcc | [ACCGACTCCAGCTCCCGAAC]AATTCACAGT | (SEQ ID NO:97) |
| | (SEQ ID NO:96) | (GTCTCCACCGCTGCCATCGC)AAGGCGGAAC | (SEQ ID NO:98) |
| mir-30c | uguaaacauccuacacucucagc | [ACCGACTCCAGCTCCCGAAC]AATGTAAACA | (SEQ ID NO:100) |
| | (SEQ ID NO:99) | (CGTCGTGAGCCATCGCCTGC)AAGCTGAGAG | (SEQ ID NO:101) |
| mir-143 | ugagaugaagcacuguagcuca | [ACCGACTCCAGCTCCCGAAC]AATGAGATGA | (SEQ ID NO:103) |
| | (SEQ ID NO:102) | (GGACGAGGAGGCCGCTTGGT)AATGAGCTAC | (SEQ ID NO:104) |
| mir-145 | guccaguuuucccaggaaucccuu | [ACCGACTCCAGCTCCCGAAC]AAGTCCAGTT | (SEQ ID NO:106) |
| | (SEQ ID NO:105) | (CCTGCTCGTCGCTCCGTTGG)AAAAGGGATT | (SEQ ID NO:107) |
| mir-196 | uagguaguuucauguuguugg | [ACCGACTCCAGCTCCCGAAC]AATAGGTAGT | (SEQ ID NO:109) |
| | (SEQ ID NO:108) | (GTGATCGCCGTCCCTGTCCG)AACCAACAAC | (SEQ ID NO:110) |
| mir-216 | uaaucucagcuggcaacugug | [ACCGACTCCAGCTCCCGAAC]AATAATCTCA | (SEQ ID NO:112) |
| | (SEQ ID NO:111) | TTCCGAGGCCTGACCCGACCAACACAGTTG | (SEQ ID NO:113) |

A series of corresponding TaqMan® probes and PNA reporter probes (shown in Table 9) are synthesized or can be obtained from commercial vendors, including without limitation, Applied Biosystems.

TABLE 9
Multiplex Probes and Second Primer Set Reverse primers.

| miRNA | | |
|---|---|---|
| target | TaqMan ® Reporter Probe | PNA Reporter Probe |
| let-7a1 | (FAM)AATGAGGTAGTAGGTTGTATAGTT-MGB | FAM-Glu-AGGTAGTAGGTT-Lys-Lys(Dabcyl) |
| | (SEQ ID NO:114) | (SEQ ID NO:115) |
| lin-4 | (FAM)AATCCCTGAGACCTCAAGTGTGA-MGB | FAM-Glu-TCCCTGAGACCTC-Lys- |
| | (SEQ ID NO:116) | Lys(Dabcyl) |
| | | (SEQ ID NO:117) |
| mir-20 | (FAM)AATAAAGTGCTTATAGTGCAGGTA(MGB) | FAM-Glu-TAAAGTGCTTATAGTG-Lys- |
| | (SEQ ID NO:118) | Lys(Dabcyl) |
| | | (SEQ ID NO:119) |
| mir-30a | (FAM)AACTTTCAGTCGGATGTTTGCAGC-MGB | FAM-Glu-CTTTCAGTCGGATG-Lys- |
| | (SEQ ID NO:120) | Lys(Dabcy) |
| | | (SEQ ID NO:121) |
| mir-7 | (FAM)AATGGAAGACTAGTGATTTTGTT(MGB | FAM-Glu-GGAAGACTAGTG-Lys-Lys(Dabcy) |
| | (SEQ ID NO:122) | (SEQ ID NO:123) |
| mir-107 | (FAM)AAAGCAGCATTGTACAGGGCTATCA-MGB | FAM-Glu-CAGCATTGTACAG-Lys- |
| | (SEQ ID NO:124) | Lys(Dabcyl) |
| | | (SEQ ID NO:125) |
| miR159a | (FAM)AATTTGGATTGAAGGGAGCTCTA-MGB | FAM-Glu-TTTGGATTGAAGG-Lys-Lys(Dabcy) |
| | (SEQ ID NO:126) | (SEQ ID NO:127) |
| mir161 | (FAM)AATTGAAAGTGACTACATCGGGG-MGB | FAM-Glu-TTGAAAGTGACTACA-Lys- |
| | (SEQ ID NO:128) | Lys(Dabcy) |
| | | (SEQ ID NO:129) |
| mir-124 | (FAM)AATAAGGCACGCGGTGAATGCCAAG- | FAM-Glu-CACGCGGTGA-Lys-Lys(Dabcyl) |
| | MGB | (SEQ ID NO:131) |
| | (SEQ ID NO:130) | |
| mir-210 | (FAM)AATTGTGCGTGTGACAGCGGCTA-MGB | FAM-Glu-TTGTGCGTGTGAC-Lys- |
| | (SEQ ID NO:132) | Lys(Dabcyl) |
| | | (SEQ ID NO:133) |
| mir-2 | (FAM)AATATCACAGCCAGCTTTGATGTGC-MGB | FAM-Glu-TCACAGCCAGCTT-Lys- |
| | (SEQ ID NO:134) | Lys(Dabcyl) |
| | | (SEQ ID NO:135) |
| mir-16 | (FAM)AATAGCAGCACGTAAATATTGGCG-MGB | FAM-Glu-AGCAGCACGTAAA-Lys- |
| | (SEQ ID NO:136) | Lys(Dabcyl) |
| | | (SEQ ID NO:137) |
| mir-21 | (FAM)AATAGCTTATCAGACTGATGTTGA-MGB | FAM-Glu-TAGCTTATCAGACTG-Lys- |
| | (SEQ ID NO:138) | Lys(Dabcyl) |
| | | (SEQ ID NO:139) |
| mir-22 | (FAM)AAAAGCTGCCAGTTGAAGAACTGT-MGB | FAM-Glu-AGCTGCCAGTTGA-Lys- |
| | (SEQ ID NO:140) | Lys(Dabcyl) |
| | | (SEQ ID NO:141) |
| mir-26a | (FAM)AATTCAAGTAATCCAGGATAGGCT-MGB | FAM-Glu-TTCAAGTAATCCAGG-Lys- |
| | (SEQ ID NO:142) | Lys(Dabcyl) |
| | | (SEQ ID NO:143) |
| mir-29 | (FAM)AACTAGCACCATCTGAAATCGGTT-MGB | FAM-Glu-CTAGCACCATCTGA-Lys- |
| | (SEQ ID NO:144) | Lys(Dabcyl) |
| | | (SEQ ID NO:145) |
| mir-34 | (FAM)AATGGCAGTGTCTTAGCTGGTTGT-MGB | FAM-Glu-TGGCAGTGTCTTAG-Lys- |
| | (SEQ ID NO:146) | Lys(Dabcyl) |
| | | (SEQ ID NO:147) |
| mir- | (FAM)AAAACATTCAACGCTGTCGGTGAGT-MGB | FAM-Glu-AACATTCAACGCTGTC-Lys- |
| 181a | (SEQ ID NO:148) | Lys(Dabcyl) |
| | | (SEQ ID NO:149) |
| mir- | (FAM)AACTCTAATACTGCCTGGTAATGATG- | FAM-Glu-CTCTAATACTGCCTGG-Lys- |
| 200b | MGB | Lys(Dabcyl) |
| | (SEQ ID NO:150) | (SEQ ID NO:151) |
| mir-223 | (FAM)AATGTCAGTTTGTCAAATACCCC-MGB | FAM-Glu-TGTCAGTTTGTCAAATA-Lys- |
| | (SEQ ID NO:152) | Lys(Dabcyl) |
| | | (SEQ ID NO:153) |
| mir-224 | (FAM)AACAAGTCACTAGTGGTTCCGTTTA-MGB | FAM-Glu-AGTCACTAGTGGT-Lys- |
| | (SEQ ID NO:154) | Lys(Dabcyl) |
| | | (SEQ ID NO:155) |
| mir-323 | (FAM)AAGCACATTACACGGTCGACCTCT-MGB | FAM-Glu-CACATTACACGGT-Lys-Lys(Dabcyl) |
| | (SEQ ID NO:156) | (SEQ ID NO:157) |
| mir- | (FAM)AACGCATCCCCTAGGGCATTGGTGT- | FAM-Glu-ATCCCCTAGGGC-Lys-Lys(Dabcyl) |
| 324-5 | MGB | (SEQ ID NO:159) |
| | (SEQ ID NO:158) | |
| mir-328 | (FAM)AACTGGCCCTCTCTGCCCTTCCGT-MGB | FAM-Glu-CTGGCCCTCTCTGC-Lys- |
| | (SEQ ID NO:160) | Lys(Dabcyl) |
| | | (SEQ ID NO:161) |
| mir-10a | (FAM)AATACCCTGTAGATCCGAATTTGTG-MGB | FAM-Glu-TACCCTGTAGATCC-Lys- |
| | (SEQ ID NO:162) | Lys(Dabcyl) |
| | | (SEQ ID NO:163) |
| mir-10b | (FAM)AATACCCTGTAGAACCGAATTTGT-MGB | FAM-Glu-TACCCTGTAGAACC-Lys- |
| | (SEQ ID NO:163) | Lys(Dabcyl) |
| | | (SEQ ID NO:164) |
| mir-23 | (FAM)AAATCACATTGCCAGGGATTTCC-MGB | FAM-Glu-ATCACATTGCCAGG-Lys- |
| | (SEQ ID NO:165) | Lys(Dabcyl) |
| | | (SEQ ID NO:166) |
| mir-27 | (FAM)AATTCACAGTGGCTAAGTTCCGCC-MGB | FAM-Glu-TTCACAGTGGCTAA-Lys- |
| | (SEQ ID NO:167) | Lys(Dabcyl) |
| | | (SEQ ID NO:168) |
| mir-30c | (FAM)AATGTAAACATCCTACACTCTCAGC-MGB | FAM-Glu-TGTAAACATCCTACACT-Lys- |
| | (SEQ ID NO:169) | Lys(Dabcyl) |
| | | (SEQ ID NO:170) |
| mir-143 | (FAM)AATGAGATGAAGCACTGTAGCTCA-MGB | FAM-Glu-AGATGAAGCACT-Lys-Lys(Dabcyl) |
| | (SEQ ID NO:171) | (SEQ ID NO:172) |
| mir-145 | (FAM)AAGTCCAGTTTTCCCAGGAATCCCTT- | FAM-Glu-CCAGTTTTCCCAGG-Lys- |
| | MGB | Lys(Dabcyl) |
| | (SEQ ID NO:173) | (SEQ ID NO:174) |
| mir-196 | (FAM)AATAGGTAGTTTCATGTTGTTGG-MGB | FAM-Glu-AGGTAGTTTCATGT-Lys- |
| | (SEQ ID NO:175) | Lys(Dabcyl) |
| | | (SEQ ID NO:176) |
| mir-216 | (FAM)AATAATCTCAGCTGGCAACTGTG-MGB | FAM-Glu-TAATCTCAGCTGGC-Lys- |
| | (SEQ ID NO:177) | Lys(Dabcyl) |
| | | (SEQ ID NO:178) |

The second primer sets, comprising the universal forward primer ACCGACTCCAGCTCCCGAAC (SEQ ID NO:179) and the corresponding reverse primers comprising unique hybridization tags (shown in Table 10), are also synthesized or obtained from commercial sources.

TABLE 10
Second Primer Set Reverse primers.

| | |
|---|---|
| miRNA | |
| Target | Second Primer Set Reverse primer |

| | | |
|---|---|---|
| let-7a1 | TTGGCCTGTTCCCGCTCGCTAA | (SEQ ID NO:180) |
| lin-4 | TCGCGGACGGTGAGCACTGCAA | (SEQ ID NO:181) |
| mir-20 | TTCCCGCTGGTGCCACTCCGAA | (SEQ ID NO:182) |
| mir-30a | CGCTCGCTTTGGCCTGCTGCAA | (SEQ ID NO:183) |
| mir-7 | CGCTGCGTGAGGGCTCGGACAA | (SEQ ID NO:184) |
| mir-107 | TTCCACCCGCGTCGTCCTGCAA | (SEQ ID NO:185) |
| miR159a | AGCACGCTTCCGGGACCCACAA | (SEQ ID NO:186) |
| mir161 | AGGCGGTGTTGGCTCGAGGCAA | (SEQ ID NO:187) |
| mir-124 | GAGGTTCCCGCTTCGCCCACAA | (SEQ ID NO:188) |
| mir-210 | GGTGCCACGTGATCGCTCCGAA | (SEQ ID NO:189) |
| mir-2 | CTGCAGGCGCGTGAGGGAGGAA | (SEQ ID NO:190) |
| mir-16 | GCCATCCGCGCTCAGTCCACAA | (SEQ ID NO:191) |
| mir-21 | GGTGGGACGCGTCTGCCTCGAA | (SEQ ID NO:192) |
| mir-22 | TCGCTTGGGTCTTCGCAGGCAA | (SEQ ID NO:193) |
| mir-26a | TTGGGACCCTCGGGACTCGCAA | (SEQ ID NO:194) |
| mir-29 | TCCGGACCAGCACTGCAGGCAA | (SEQ ID NO:195) |
| mir-34 | TCGCGAGGGTCTTCCGGAGGAA | (SEQ ID NO:196) |
| mir-181a | TGGTCCACCCTGCTGCTCGCAA | SEQ ID NO:197 |
| mir-200b | TCGCGACCGTCTGCTCAGGCAA | (SEQ ID NO:198) |
| mir-223 | CGCTGGACCGCTAGGCACCCAA | (SEQ ID NO:199) |
| mir-224 | TTGGGCGTTGGTGCGTCCTGAA | (SEQ ID NO:200) |
| mir-323 | GCTCCAGTTCCGGGTGCGCTAA | (SEQ ID NO:201) |
| mir-324-5 | GCGTGAGGTTGGGAGGGCGTAA | (SEQ ID NO:202) |
| mir-328 | TTGGTTGGGAGGCTGCGGTGAA | (SEQ ID NO:203) |
| mir-10a | CGTCGCTCTGGTTCGCTCGCAA | (SEQ ID NO:204) |
| mir-10b | GGTGACCCGTGACGCTGCCAAA | (SEQ ID NO:205) |
| mir-23 | AGGCTTGGCGCTCGTCACCCAA | (SEQ ID NO:206) |
| mir-27 | GTCTCCACCGCTGCCATCGCAA | (SEQ ID NO:207) |
| mir-30c | CGTCGTGAGCCATCGCCTGCAA | (SEQ ID NO:208) |
| mir-143 | GGACGAGGAGGCCGCTTGGTAA | (SEQ ID NO:209) |
| mir-145 | CCTGCTCGTCGCTCCGTTGGAA | (SEQ ID NO:210) |
| mir-196 | GTGATCGCCGTCCCTGTCCGAA | (SEQ ID NO:211) |
| mir-216 | TTCCGAGGCCTGACCCGACCAA | (SEQ ID NO:212) |

A TaqMan Low Density Array (Applied Biosystems) is prepared by pre-spotting individual chambers along the same fill port channel with reverse primers of the second primer set and corresponding reporter probe for each miRNA target. Typically, 1 μL of a solution comprising a reverse primer and corresponding reporter probe is spotted in the appropriate chamber of a Low Density Array card and dried, and then the card is sealed.

The two phase multiplex is performed as follows. A first reaction composition is formed comprising: 1 μL total RNA sample (typically 0.1-100 ng), 1 μL of a mixture of the 33 first primer sets (0.5 μM), 5 μL 2× RT-PCR Master Mix (Applied Biosystems), 2.75 μL ddH₂O, and 0.25 μL 40× MultiScribe™ Reverse Transcriptase with RNA inhibitor (Applied Biosystems). This first reaction composition is incubated at 20° C. for 20 minutes, 37° C. for 30 minutes, 95° C. for ten minutes, cycled ten times (95° C./15 seconds, 60° C. for 1 minute), then cooled to 4° C. Ten μL of this reacted first reaction composition is combined with 8 μL universal forward primer (10 μM), 40 μL 2× TaqMan® Universal Master Mix (Applied Biosystems), and 23 μL ddH₂O. 80 μL of this mixture is placed in the appropriate loading port of the pre-spotted TaqMan® Low Density Array which is then loaded into a ABI PRISM® 7900HT Sequence Detection System (Applied Biosystems). The remainder of the assay is performed according to the 7900HT users instructions. The fluorescence from each chamber is detected as the assay cycles, the threshold is determined by the integrated software, and C_{T} values are generated. Based on the corresponding C_{T} value, the initial concentration of each synthetic miRNA target can be quantitated using standard curves.

### EXAMPLE 7

### Exemplary Method

A first reaction composition comprising 1 μL (10 ng/μL) Mouse Lung Total RNA (Stratagene), 1 μL let-7a1-specific reverse primer (1 μM) with the sequence GTGTCGTGGAGTCGGCAAAACTATAC (SEQ ID NO:213) comprising a target-binding portion including eight nucleotides (shown underlined) and an upstream primer-binding portion, 5 μL 2× RT-PCR Master Mix No AmpErase® UNG (Applied Biosystems), 2.75 μL ddH₂O, and 0.25 μL MultiScribe™ Reverse Transcriptase with 40× RNase inhibitor (Applied Biosystems) was formed. The first reaction composition was heated to 20° C. for 20 minutes, 37° C. for thirty minutes, 85° C. for five minutes, and then cooled to 4° C.

A second reaction composition was formed by adding 1 μL of the corresponding let-7a1 forward primer (1 μM; SEQ ID NO:16 in Table 8) to the reacted first reaction composition. The second reaction composition was heated to 95° C. for ten minutes, cycled ten times at (95° C. for 15 seconds, 60° C. for one minute), then the reacted second reaction composition was cooled to 4° C.

A third reaction composition was formed comprising 2 μL of the reacted second reaction composition, a corresponding second primer set (1 μL first primer (10 μM) with the sequence ACCGACTCCAGCTCCCGAAC (SEQ ID NO:214) and 1 μL second primer (10 μM) with the sequence GTGTCGTGGAGTCGGCAA (SEQ ID NO:215), 1 μL reporter probe (5 μM; SEQ ID NO:114 in Table 9), and 5μL 2× TaqMan® Universal Master Mix (Applied Biosystems). The third reaction composition was manually pipetted into a well of a 384 well plate and loaded into an ABI PRISM® 7900HT Sequence Detection System (Applied Biosystems). The third reaction composition was heated to 95° C. for ten minutes then cycled 40 times at (95° C. for 15 seconds and 60° C. for one minute) and a Ct value of 31.79 was obtained for let-7a1.

### EXAMPLE 8

### Exemplary Method

A first reaction composition comprising 1 μL (10 ng/μL) Mouse Lung Total RNA (Stratagene), 1 μL SEQ ID NO:16 as the forward primer (1 μM), 1 μL SEQ ID NO:213 as the reverse primer (1 μM), 5 μL 2× RT-PCR Master Mix No AmpErase® UNG (Applied Biosystems), 1.75 μL ddH₂O, and 0.25 μL MultiScribe™ Reverse Transcriptase with 40× RNase inhibitor (Applied Biosystems) was formed. The first reaction composition was heated to 20° C. for twenty minutes, 37° C. for thirty minutes, 85° C. for five minutes, then the reacted first reaction composition was cooled to 4° C.

A second reaction composition was formed comprising 2 μL of the reacted first reaction composition, 1 μL forward primer and 1 μL reverse primer of the second primer set of Example 7, 1 μL reporter probe (5 μM; SEQ ID NO:114 in Table 9), and 5 μL 2× TaqMan® Universal Master Mix (Applied Biosystems). The second reaction composition was manually pipetted into a well of a 384 well plate and loaded into an ABI PRISM® 7900HT Sequence Detection System (Applied Biosystems). The second reaction composition was heated to 95° C. for ten minutes then cycled 40 times at (95° C. for 15 seconds and 60° C. for one minute) and a Ct value of 21.60 was obtained for let-7a1.

### EXAMPLE 9

### Exemplary Method

A first reaction composition was formed as described in Example 8 and reacted as follows: 20° C. for 20 minutes, 37° C. for 30 minutes, 95° C. for ten minutes, cycled three times at (95° C. for 15 seconds and 40° C. for one minute), then cooled to 4° C.

A second reaction composition was formed comprising 2 μL of the reacted first reaction composition, the second primer set of Example 7, 1 μL reporter probe (5 μM; SEQ ID NO:114 in Table 9), and 5 μL 2× TaqMan® Universal Master Mix (Applied Biosystems). The second reaction composition was manually pipetted into a well of a 384 well plate and loaded into an ABI PRISM® 7900HT Sequence Detection System (Applied Biosystems). The second reaction composition was heated to 95° C. for ten minutes then cycled 40 times at (95° C. for 15 seconds and 60° C. for one minute) and a Ct value of 17.39 was obtained for let-7a1.

### EXAMPLE 10

### Exemplary Method

A first reaction composition was formed as described and reacted as in Example 7. A second reaction composition was formed and reacted as described in Example 7, except that the cycling step was three cycles of (95° C. for 15 seconds, then 40° C. for one minute).

A third reaction composition was formed comprising 2 μL of the reacted second reaction composition, 1 μL of the first primer 1 μL of the second primer of the second primer set of Example 7, 1 μL of the reporter probe of Example 7, and 5 μL 2× TaqMan® Universal Master Mix (Applied Biosystems). The third reaction composition was manually pipetted into a well of a 384 well plate and loaded into an ABI PRISM® 7900HT Sequence Detection System (Applied Biosystems). The third reaction composition was heated to 95° C. for ten minutes then cycled 40 times at (95° C. for 15 seconds and 60° C. for one minute) and a Ct value of 17.20 was obtained for let-7a1.

### EXAMPLE 11

### Exemplary Method

A first reaction composition was formed and reacted as described in Example 7. A second reaction composition was formed and reacted as described in Example 7, except that the cycling step was three cycles of (95° C. for 15 seconds, then 40° C. for one minute), then seven cycles of (95° C. for 15 seconds and 60° C. for one minute.

A third reaction composition was formed comprising 2 μL of the reacted second reaction composition, the second primer set of Example 7, the reporter probe of Example 7, and 5 μL 2× TaqMan® Universal Master Mix (Applied Biosystems). The third reaction composition was manually pipetted into a well of a 384 well plate and loaded into an ABI PRISM® 7900HT Sequence Detection System (Applied Biosystems). The third reaction composition was heated to 95° C. for ten minutes then cycled 40 times at (95° C. for 15 seconds and 60° C. for one minute) and a Ct value of 11.47 was obtained for let-7a1.

Although the disclosed teachings has been described with reference to various applications, methods, and compositions, it will be appreciated that various changes and modifications may be made without departing from the teachings herein. The foregoing examples are provided to better illustrate the disclosed teachings and are not intended to limit the scope of the teachings herein.

We claim:

1. A primer comprising a target-binding portion and a second portion, wherein the second portion is upstream from the target-binding portion and the target-binding portion comprises no more than ten nucleotides that have the same sequence as a region of a corresponding target.

2. The primer of claim 1, wherein the target-binding portion comprises six, seven, eight, or nine nucleotides.

3. The primer of claim 2, wherein the second portion comprises a primer-binding portion.

4. The primer of claim 1, wherein the corresponding target is a polynucleotide.

5. The primer of claim 4, wherein the polynucleotide target is a small RNA molecule.

6. A primer comprising a target-binding portion and a second portion, wherein the second portion is upstream from the target-binding portion and the target-binding portion comprises no more than ten nucleotides that have a sequence that is complementary to a region of a corresponding target.

7. The primer of claim 6, wherein the target-binding portion comprises six, seven, eight, or nine nucleotides.

8. The primer of claim 7, wherein the second portion comprises a primer-binding portion.

9. The primer of claim 6, wherein the corresponding target is a polynucleotide.

10. The primer of claim 9, wherein the polynucleotide target is a small RNA molecule.

11. A primer set comprising:
a forward primer that comprises a primer-binding portion and a target-binding portion, wherein the primer-binding portion is upstream from the target-binding portion and the target-binding portion comprises no more than ten nucleotides that have the same sequence as a first region of a corresponding target; and
a reverse primer that comprises a primer-binding portion and a target-binding portion, wherein the primer-binding portion is upstream from the target-binding portion and the target-binding portion comprises no more than ten nucleotides that have a sequence that is complementary to a second region of the corresponding target.

12. The primer set of claim 11, wherein the target-binding portion of the forward primer comprises six, seven, eight, or nine nucleotides.

13. The primer set of claim 11, wherein the target-binding portion of the reverse primer comprises six, seven, eight, or nine nucleotides.

14. The primer set of 13, wherein the target-binding portion of the forward primer comprises six, seven, eight, or nine nucleotides.

15. A reporter probe comprising: at least two deoxyribonucleotides upstream from at least four peptide nucleic acids (PNAs); and a reporter group.

16. The reporter probe of claim 15, wherein the reporter group comprises a fluorescent reporter group and a quencher.

17. The reporter probe of claim 15, further comprising a minor groove binder.

18. A method for identifying a small RNA molecule comprising:
hybridizing a reverse primer of a first primer set to the small RNA molecule, wherein the reverse primer comprises: (a) a primer-binding portion that is upstream from (b) a small RNA molecule-binding portion comprising no more than ten nucleotides that are complementary to a second region of the small RNA molecule;
extending the hybridized reverse primer with a first extending enzyme to generate a reverse-transcribed product;
hybridizing a forward primer of the first primer set to the reverse-transcribed product, wherein the forward primer comprises: (a) a primer-binding portion that is upstream from (b) a small RNA molecule-binding portion comprising no more than ten nucleotides having the same sequence as a first region of the small RNA molecule;
extending the hybridized forward primer with a second extending enzyme to generate a first amplicon;
amplifying the first amplicon to generate an additional first amplicon;
combining the additional first amplicon with a second primer set;
amplifying the additional first amplicons to generate second amplicons;
detecting the second amplicons; and
identifying the small RNA molecule.

19. The method of claim 18, wherein the first extending enzyme and the second extending enzyme are the same enzyme.

20. The method of claim 18, wherein the first extending enzyme and the second extending enzyme are different enzymes.

21. The method of claim 18, wherein the combining further comprises a third extending enzyme.

22. The method of claim 21, wherein (a) the second extending enzyme and the third extending enzyme are the same enzyme and (b) the first extending enzyme and the second extending enzyme are different enzymes.

23. The method of claim 18, wherein the generating the second amplicons and the detecting comprise a real-time instrument.

24. The method of claim 18, wherein the small RNA molecule comprises a microRNA (miRNA), a small interfering RNA (siRNA), or a miRNA and a siRNA.

25. The method of claim 18, wherein the small RNA molecule-binding portion of the forward primer comprises six, seven, eight, or nine nucleotides having the same sequence as the first region of the small RNA molecule.

26. The method of claim 18, wherein the small RNA molecule-binding portion of the reverse primer comprises six, seven, eight, or nine nucleotides that are complementary to the second region of the small RNA molecule.

27. The method of claim 26, wherein the small RNA molecule-binding portion of the forward primer comprises six, seven, eight, or nine nucleotides having the same sequence as the first region of the small RNA molecule.

28. The method of claim 18, wherein a primer of the second primer set comprises a universal priming sequence, a hybridization tag, or a universal priming sequence and a hybridization tag.

29. The method of claim 28, wherein both the first primer and the second primer of the second primer set comprise a universal priming sequence, a hybridization tag or a universal priming sequence and a hybridization tag, wherein the universal priming sequences are the same or different and the hybridization tags are the same or different.

30. The method of claim 18, wherein a second amplicon comprises an affinity tag, a reporter group, a mobility modifier, a hybridization tag, or combinations thereof.

31. The method of claim 18, wherein the detecting comprises a mobility-dependent analytical technique.

32. The method of claim 18, wherein the detecting further comprises a reporter probe.

33. The method of claim 32, wherein the reporter probe comprises a fluorescent reporter group, a quencher, a minor groove binder, or combinations thereof.

34. The method of claim 33, wherein the reporter probe comprises: (a) a nucleotide or nucleotide analog that has the same nucleotide base as the 3′-end of the small RNA molecule-binding portion of the forward primer or is complementary to the 3′-end of the small RNA molecule-binding portion of the forward primer; adjacent to (b) at least two nucleotides or nucleotide analogs that have the same nucleotide bases as or are complementary to at least two nucleotides of the small RNA molecule and that are not the same as or complementary to the small RNA molecule-binding portion of the forward primer or the small RNA molecule-binding portion of the reverse primer; adjacent to (c) a nucleotide or nucleotide analog that has the same nucleotide base as the 3′-end of the small RNA molecule-binding portion of the reverse primer or is complementary to the 3′-end of the small RNA molecule-binding portion of the reverse primer.

35. The method of claim 33, wherein the reporter probe sequence is not the same as or complementary to either (a) the small RNA molecule-binding portion of the forward primer or (b) the small RNA molecule-binding portion of the reverse primer.

36. The method of claim 18, wherein: the small RNA molecule comprises a multiplicity of different small RNA molecules; the first primer set comprises a multiplicity of different first primer sets; and the detecting comprises detecting a multiplicity of different second amplicons.

37. The method of claim 18, wherein the small RNA molecule comprises 17 to 29 ribonucleotides.

38. The method of claim 36, wherein the small RNA molecule comprises a miRNA, a siRNA, or a miRNA and a siRNA.

39. The method of claim 18, wherein the small RNA molecule comprises less than 100 ribonucleotides.

40. The method of claim 39, wherein the small RNA molecule comprises a miRNA precursor.

41. A method for identifying a small RNA molecule comprising:
hybridizing a reverse primer of a first primer set to the small RNA molecule, wherein the reverse primer comprises a primer-binding portion and a small RNA molecule-binding portion comprising six, seven, eight, or nine nucleotides that are complementary to a second region of the small RNA molecule;
extending the hybridized reverse primer with a first extending enzyme to generate a reverse-transcribed product;
hybridizing a forward primer of the first primer set to the reverse-transcribed product, wherein the forward primer comprises a primer-binding portion and a small RNA molecule-binding portion comprising six, seven, eight, or nine nucleotides that are the same as a first region of the small RNA molecule;
extending the hybridized forward primer with a second extending enzyme to generate a first amplicon;
amplifying the first amplicons using the first primer set to generate additional first amplicons;
combining the additional first amplicons, a third extending enzyme, and a second primer set comprising a first primer and a second primer, wherein the first primer comprises a first universal priming sequence, the second primer comprises a second universal priming sequence, or one primer comprises a universal priming sequence and the other primer comprises a unique hybridization tag;
amplifying the additional first amplicons to generate second amplicons;
detecting the second amplicons using a reporter probe comprising a fluorescent reporter group, a quencher, a minor groove binder, or combinations thereof; and
identifying the small RNA molecule.

42. The method of claim 41, wherein the second extending enzyme and the third extending enzyme are the same enzyme; and the first extending enzyme and the second extending enzyme are different enzymes.

43. The method of claim 41, wherein the reporter probe comprises: (a) a nucleotide or nucleotide analog that has the same nucleotide base as the 3′-end of the small RNA molecule-binding portion of the forward primer or is complementary to the 3′-end of the small RNA molecule-binding portion of the forward primer; adjacent to (b) at least two nucleotides or nucleotide analogs that have the same nucleotide bases as or are complementary to at least two nucleotides of the small RNA molecule and that are not the same as or complementary to the small RNA molecule-binding portion of the forward primer or the small RNA molecule-binding portion of the reverse primer; adjacent to (c) a nucleotide or nucleotide analog that has the same nucleotide base as the 3′-end of the small RNA molecule-binding portion of the reverse primer or is complementary to the 3′-end of the small RNA molecule-binding portion of the reverse primer.

44. The method of claim 41, wherein the reporter probe sequence is not the same as or complementary to either (a) the small RNA molecule-binding portion of the forward primer or (b) the small RNA molecule-binding portion of the reverse primer.

45. The method of claim 41, wherein: the small RNA molecule comprises a multiplicity of different small RNA molecules; the first primer set comprises a multiplicity of different first primer sets; the reporter probe comprises a multiplicity of different reporter probes; and the detecting comprises detecting a multiplicity of different second amplicons.

46. The method of claim 41, wherein the small RNA molecule comprises 17 to 29 ribonucleotides.

47. The method of claim 46, wherein the small RNA molecule comprises a miRNA, a siRNA, or a miRNA and a siRNA.

48. A method for identifying a small RNA molecule comprising:
combining the small RNA molecule with a first primer set, a second primer set, first extending enzyme, and optionally, a second extending enzyme, wherein the first primer set comprises: (a) a forward primer comprising (i) a primer-binding portion that is upstream from (ii) a small RNA molecule-binding portion comprising no more than ten nucleotides having the same sequence as a first region of the small RNA molecule and (b) a reverse primer comprising (i) a primer-binding portion that is upstream from (ii) a small RNA molecule-binding portion comprising no more than ten nucleotides that are complementary to a second region of the small RNA molecule;
generating a reverse-transcribed product, a first amplicon, an additional first amplicon, and a second amplicon;
detecting the second amplicon; and
identifying the small RNA molecule.

49. The method of claim 48, wherein the small RNA molecule-binding portion of the forward primer comprises six, seven, eight, or nine nucleotides having the same sequence as the first region of the small RNA molecule.

50. The method of claim 48, wherein the small RNA molecule-binding portion of the reverse primer comprises six, seven, eight, or nine nucleotides that are complementary to the second region of the small RNA molecule.

51. The method of claim 50, wherein the small RNA molecule-binding portion of the forward primer comprises six, seven, eight, or nine nucleotides having the same sequence as the first region of the small RNA molecule.

52. The method of claim 48, wherein a primer of the second primer set comprises a universal priming sequence, a hybridization tag, or a universal priming sequence and a hybridization tag.

53. The method of claim 52, wherein both the first primer and the second primer of the second primer set comprise a universal priming sequence, a hybridization tag, or a universal priming sequence and a hybridization tag, wherein the universal priming sequences are the same or different and the hybridization tags are the same or different.

54. The method of claim 48, wherein a second amplicon comprises an affinity tag, a reporter group, a mobility modifier, a hybridization tag, or combinations thereof.

55. The method of claim 48, wherein the detecting comprises a mobility-dependent analytical technique.

56. The method of claim 48, wherein the combining further comprises a reporter probe.

57. The method of claim 56, wherein the reporter probe comprises a fluorescent reporter group, a quencher, a minor groove binder, or combinations thereof.

58. The method of claim 57, wherein the reporter probe comprises: (a) a nucleotide or nucleotide analog that has the same nucleotide base as the 3′-end of the small RNA molecule-binding portion of the forward primer or is complementary to the 3′-end of the small RNA molecule-binding portion of the forward primer; adjacent to (b) at least two nucleotides or nucleotide analogs that have the same nucleotide bases as or are complementary to at least two nucleotides of the small RNA molecule and that are not the same as or complementary to the small RNA molecule-binding portion of the forward primer or the small RNA molecule-binding portion of the reverse primer; adjacent to (c) a nucleotide or nucleotide analog that has the same nucleotide base as the 3′-end of the small RNA molecule-binding portion of the reverse primer or is complementary to the 3′-end of the small RNA molecule-binding portion of the reverse primer.

59. The method of claim 57, wherein the reporter probe sequence is not the same as or complementary to either (a) the small RNA molecule-binding portion of the forward primer or (b) the small RNA molecule-binding portion of the reverse primer.

60. The method of claim 48, wherein: the small RNA molecule comprises a multiplicity of different small RNA molecules; the first primer set comprises a multiplicity of different first primer sets; and the detecting comprises detecting a multiplicity of different second amplicons.

61. The method of claim 48, wherein the small RNA molecule comprises 17 to 29 ribonucleotides.

62. The method of claim 61, wherein the small RNA molecule comprises a miRNA, a siRNA, or a miRNA and a siRNA.

63. A method for identifying a small RNA molecule comprising:
combining the small RNA molecule with a first primer set, a second primer set, a reporter probe, a first extending enzyme, and optionally, a second extending enzyme, wherein (a) the first primer set comprises (1) a forward primer comprising (i) a primer-binding portion that is upstream from (ii) a small RNA molecule-binding portion comprising six, seven, eight, or nine nucleotides having the same sequence as a first region of the small RNA molecule and (2) a reverse primer comprising (i) a primer-binding portion that is upstream from (ii) a small RNA molecule-binding portion comprising six, seven, eight, or nine nucleotides that are complementary to a second region of the small RNA molecule; (b) the second primer set comprises a first primer and a second primer, wherein the first primer, the second primer, or the first primer and the second primer comprise a universal priming sequence, a hybridization tag, or a universal priming sequence and a hybridization tag; and (c) the reporter probe comprises a fluorescent reporter group, a quencher, a minor groove binder, or combinations thereof; and wherein the reporter probe sequence is not the same as or complementary to either (a) the small RNA molecule-binding portion of the forward primer or (b) the small RNA molecule-binding portion of the reverse primer;
generating a reverse-transcribed product, a first amplicon, an additional first amplicon, and a second amplicon;
detecting the second amplicons; and
identifying the small RNA molecule.

64. The method of claim 63, wherein the extending enzyme comprises a reverse transcriptase and a DNA polymerase.

65. The method of claim 63, wherein: the small RNA molecule comprises a multiplicity of different small RNA molecules; the first primer set comprises a multiplicity of different first primer sets; the reporter probe comprises a multiplicity of different reporter probes; and the detecting comprises detecting a multiplicity of different second amplicons.

66. The method of claim 63, wherein the small RNA molecule comprises 17 to 29 ribonucleotides.

67. The method of claim 66, wherein the small RNA molecule comprises a miRNA, a siRNA, or a miRNA and a siRNA.

68. The method of claim 63, wherein the small RNA molecule comprises less than 100 ribonucleotides.

69. The method of claim 68, wherein the small RNA molecule comprises a miRNA precursor.

70. A method for quantitating a small RNA molecule comprising:
hybridizing a reverse primer of a first primer set to the small RNA molecule, wherein the reverse primer comprises: (a) a primer-binding portion that is upstream from (b) a small RNA molecule-binding portion comprising no more than ten nucleotides that are complementary to a second region of the small RNA molecule;
extending the hybridized reverse primer with a first extending enzyme to generate a reverse-transcribed product;
hybridizing a forward primer of the first primer set to the reverse-transcribed product, wherein the forward primer comprises: (a) a primer-binding portion that is upstream from (b) a small RNA molecule-binding portion comprising no more than ten nucleotides having the same sequence as a first region of the small RNA molecule;
extending the hybridized forward primer with a second extending enzyme to generate a first amplicon;
amplifying the first amplicon to generate an additional first amplicon;
combining the additional first amplicon with a second primer set;
amplifying the additional first amplicon to generate a second amplicon;
detecting the second amplicon; and
quantitating the small RNA molecule.

71. The method of claim 70, wherein the first extending enzyme and the second extending enzyme are the same enzyme.

72. The method of claim 70, wherein the first extending enzyme and the second extending enzyme are different enzymes.

73. The method of claim 70, wherein the combining further comprises a third extending enzyme.

74. The method of claim 73, wherein (a) the second extending enzyme and the third extending enzyme are the same enzyme and (b) the first extending enzyme and the second extending enzyme are different enzymes.

75. The method of claim 70, wherein the generating the second amplicon, the detecting, and the quantitating comprise a real-time instrument.

76. The method of claim 70, wherein the small RNA molecule comprises a miRNA, a siRNA, or an miRNA and a siRNA.

77. The method of claim 70, wherein the small RNA molecule-binding portion of the forward primer comprises six, seven, eight, or nine nucleotides having the same sequence as the first region of the small RNA molecule.

78. The method of claim 70, wherein the small RNA molecule-binding portion of the reverse primer comprises six, seven, eight, or nine nucleotides that are complementary to the second region of the small RNA molecule.

79. The method of claim 78, wherein the small RNA molecule-binding portion of the forward primer comprises six, seven, eight, or nine nucleotides having the same sequence as the first region of the small RNA molecule.

80. The method of claim 70, wherein a primer of the second primer set comprises a universal priming sequence, a hybridization tag, or a universal priming sequence and a hybridization tag.

81. The method of claim 80, wherein both the first primer and the second primer of the second primer set comprise a universal priming sequence, a hybridization tag, or a universal priming sequence and a hybridization tag, wherein the universal priming sequences are the same or different and the hybridization tags are the same or different.

82. The method of claim 70, wherein a second amplicon comprises an affinity tag, a reporter group, a mobility modifier, a hybridization tag, or combinations thereof.

83. The method of claim 70, wherein the detecting comprises a mobility-dependent analytical technique.

84. The method of claim 70, wherein the detecting further comprises a reporter probe.

85. The method of claim 84, wherein the reporter probe further comprises: a deoxyribonucleotide, a ribonucleotide, a PNA, a locked nucleic acid (LNA), a 2′-O-alkyl nucleotide, a phosphoroamidate, a fluoro-arabino nucleic acid (FANA), a morpholino phosphoroamidate (MP), a cyclohexene nucleic acid (CeNA), a tricyclo DNA (tcDNA),or combinations thereof.

86. The method of claim 84, wherein the reporter probe further comprises a multiplicity of: deoxyribonucleotides, ribonucleotides, PNAs, LNAs, 2′-O-alkyl nucleotides, phosphoroamidates, FANAs, MPs, CeNAs, tcDNAs, but not combinations thereof.

87. The method of claim 84, wherein the reporter probe comprises a fluorescent reporter group, a quencher, a minor groove binder, or combinations thereof.

88. The method of claim 87, wherein the reporter probe comprises: (a) a nucleotide or nucleotide analog that has the same nucleotide base as the 3′-end of the small RNA molecule-binding portion of the forward primer or is complementary to the 3′-end of the small RNA molecule-binding portion of the forward primer; adjacent to (b) at least two nucleotides or nucleotide analogs that have the same nucleotide bases as or are complementary to at least two nucleotides of the small RNA molecule and that are not the same as or complementary to the small RNA molecule-binding portion of the forward primer or the small RNA molecule-binding portion of the reverse primer; adjacent to (c) a nucleotide or nucleotide analog that has the same nucleotide base as the 3′-end of the small RNA molecule-binding portion of the reverse primer or is complementary to the 3′-end of the small RNA molecule-binding portion of the reverse primer.

89. The method of claim 87, wherein the reporter probe sequence is not the same as or complementary to either (a) the small RNA molecule-binding portion of the forward primer or (b) the small RNA molecule-binding portion of the reverse primer.

90. The method of claim 70, wherein: the small RNA molecule comprises a multiplicity of different small RNA molecules; the first primer set comprises a multiplicity of different first primer sets; and the detecting comprises detecting a multiplicity of different second amplicons.

91. The method of claim 70, wherein the small RNA molecule comprises 17 to 29 ribonucleotides.

92. The method of claim 91, wherein the small RNA molecule comprises a miRNA, a siRNA, or a miRNA and a siRNA.

93. The method of claim 70, wherein the small RNA molecule comprises less than 100 ribonucleotides.

94. The method of claim 93, wherein the small RNA molecule comprises a miRNA precursor.

95. The method of claim 70, wherein the amplifying, the detecting, and the quantitating comprise quantitative PCR (Q-PCR); and the combining further comprises a reporter probe.

96. The method of claim 95, wherein the reporter probe comprises a fluorescent reporter group, a quencher, a minor groove binder, or combinations thereof.

97. The method of claim 95, wherein the reporter probe further comprises: a deoxyribonucleotide, a ribonucleotide, a PNA, a LNA, a 2′-O-alkyl nucleotide, a phosphoroamidate, a FANA, a MP, a CeNA, a tcDNA, or combinations thereof.

98. The method of claim 97, wherein the reporter probe comprises: at least two deoxyribonucleotides that are upstream from at least four PNAs; a fluorescent reporter group; and a quencher.

99. The method of claim 98, wherein the reporter probe further comprises a minor groove binder.

100. The method of claim 96, wherein the reporter probe further comprises a multiplicity of: deoxyribonucleotides, ribonucleotides, PNAs, LNAs, 2′-O-alkyl nucleotides, phosphoroamidates, FANAs, MPs, CeNAs, tcDNAs, but not combinations thereof.

101. The method of claim 96, wherein the reporter probe comprises: (a) a nucleotide or nucleotide analog that has the same nucleotide base as the 3′-end of the small RNA molecule-binding portion of the forward primer or is complementary to the 3′-end of the small RNA molecule-binding portion of the forward primer; adjacent to (b) at least two nucleotides or nucleotide analogs that have the same nucleotide bases as or are complementary to at least two nucleotides of the small RNA molecule and that are not the same as or complementary to the small RNA molecule-binding portion of the forward primer or the small RNA molecule-binding portion of the reverse primer; adjacent to (c) a nucleotide or nucleotide analog that has the same nucleotide base as the 3′-end of the small RNA molecule-binding portion of the reverse primer or is complementary to the 3′-end of the small RNA molecule-binding portion of the reverse primer.

102. The method of claim 96, wherein the reporter probe sequence is not the same as or complementary to either (a) the small RNA molecule-binding portion of the forward primer or (b) the small RNA molecule-binding portion of the reverse primer.

103. The method of claim 96, wherein: (a) the small RNA molecule comprises a multiplicity of different small RNA molecules; (b) the first primer set comprises a multiplicity of different first primer sets; (c) the detecting comprises detecting a multiplicity of different second amplicons; and (d) the Q-PCR comprises performing a multiplicity of different assays, wherein an assay comprises (i) a second primer set, (ii) a reporter probe, (iii) and an aliquot of the additional first amplicons; and wherein at least one of the multiplicity of different assays is designed to quantitate a subset of the multiplicity of different small RNA molecules.

104. The method of claim 70, wherein the small RNA molecule comprises a multiplicity of different small RNA molecules.

105. A method for quantitating a multiplicity of small RNA molecules comprising:
hybridizing a multiplicity of different reverse primers from a multiplicity of different first primer sets to a multiplicity of different RNA molecules, wherein the different reverse primers each comprise: (a) a primer-binding portion that is upstream from (b) a small RNA molecule-binding portion comprising six, seven, eight, or nine nucleotides that are complementary to a second region of corresponding small RNA molecules;
extending the multiplicity of different hybridized reverse primers with a first extending enzyme to generate a multiplicity of reverse-transcribed products;
hybridizing a multiplicity of different forward primers from the multiplicity of first primer sets to the multiplicity of reverse-transcribed products, wherein at least one of the different forward primers comprise: (a) a primer-binding portion that is upstream from (b) a small RNA molecule-binding portion comprising six, seven, eight, or nine nucleotides having the same sequence as a first region of corresponding small RNA molecules;
extending the multiplicity of hybridized forward primers with a second extending enzyme to generate a multiplicity of different first amplicons;
amplifying the multiplicity of different first amplicons using the first primer set to generate a multiplicity of different additional first amplicons;
performing a multiplicity of different assays, wherein an assay comprises (a) a second primer set, comprising a first primer and a second primer, wherein the first primer, the second primer, or the first primer and the second primer comprise a universal priming sequence, a hybridization tag, or a universal priming sequence and a hybridization tag; (b) a reporter probe, an intercalating agent, or a reporter probe and an intercalating agent, wherein the reporter probe comprises a fluorescent reporter group, a quencher, a minor groove binder, or combinations thereof; (c) an aliquot of the multiplicity of additional first amplicons; and (d) a third extending enzyme; and
quantitating the multiplicity of different small RNA molecules.

106. The method of claim 105, wherein (a) the second extending enzyme and the third extending enzyme are the same enzyme or different enzymes, and (b) the first extending enzyme and the second extending enzyme are the same enzyme or different enzymes.

107. The method of claim 105, wherein the reporter probe comprises: (a) a nucleotide or nucleotide analog that has the same nucleotide base as the 3′-end of the small RNA molecule-binding portion of the forward primer or is complementary to the 3′-end of the small RNA molecule-binding portion of the forward primer; adjacent to (b) at least two nucleotides or nucleotide analogs that have the same nucleotide bases as or are complementary to at least two nucleotides of the small RNA molecule and that are not the same as or complementary to the small RNA molecule-binding portion of the forward primer or the small RNA molecule-binding portion of the reverse primer; adjacent to (c) a nucleotide or nucleotide analog that has the same nucleotide base as the 3′-end of the small RNA molecule-binding portion of the reverse primer or is complementary to the 3′-end of the small RNA molecule-binding portion of the reverse primer.

108. The method of claim 105, wherein the reporter probe sequence is not the same as or complementary to either (a) the small RNA molecule-binding portion of the forward primer or (b) the small RNA molecule-binding portion of the reverse primer.

109. The method of claim 105, wherein the small RNA molecule comprises 17 to 29 ribonucleotides.

110. The method of claim 109, wherein the small RNA molecule comprises a miRNA, a siRNA, or a miRNA and a siRNA.

111. The method of claim 105, wherein the multiplicity of different assays comprise Q-PCR.

112. A method for quantitating a small RNA molecule comprising:
combining the small RNA molecule with a first primer set, a second primer set, a first extending enzyme, and optionally, a second extending enzyme, wherein the first primer set comprises: (a) a forward primer comprising (i) a primer-binding portion that is upstream from (ii) a small RNA molecule-binding portion that includes no more than ten nucleotides having the same sequence as a first region of the small RNA molecule and (b) a reverse primer comprising (i) a primer-binding portion that is upstream from (ii) a small RNA molecule-binding portion that includes no more than ten nucleotides that are complementary to a second region of the small RNA molecule;
generating a reverse-transcribed product, a first amplicon, an additional first amplicon, and a second amplicon;
detecting the second amplicons;
and quantitating the small RNA molecule.

113. The method of claim 112, wherein the extending enzyme comprises a reverse transcriptase and a DNA polymerase.

114. The method of claim 112, wherein the small RNA molecule-binding portion of the forward primer comprises six, seven, eight, or nine nucleotides having the same sequence as the first region of the small RNA molecule.

115. The method of claim 112, wherein the small RNA molecule-binding portion of the reverse primer comprises six, seven, eight, or nine nucleotides that are complementary to the second region of the small RNA molecule.

116. The method of claim 115, wherein the small RNA molecule-binding portion of the forward primer comprises six, seven, eight, or nine nucleotides having the same sequence as the first region of the small RNA molecule.

117. The method of claim 112, wherein a primer of the second primer set comprises a universal priming sequence, a hybridization tag, or a universal priming sequence and a hybridization tag.

118. The method of claim 112, wherein both the first primer and the second primer of the second primer set comprise a universal priming sequence, a hybridization tag, or a universal priming sequence and a hybridization tag, wherein the universal priming sequences are the same or different and the hybridization tags are the same or different.

119. The method of claim 112, wherein the detecting further comprises a reporter probe.

120. The method of claim 119, wherein the reporter probe comprises a fluorescent reporter group, a quencher, a minor groove binder, or combinations thereof.

121. The method of claim 120, wherein the reporter probe comprises: (a) a nucleotide or nucleotide analog that is the same as the 3′-end of the small RNA molecule-binding portion or is complementary to the 3′-end of the small RNA molecule-binding portion of the forward primer; adjacent to (b) at least two nucleotides or nucleotide analogs that are the same as or are complementary to at least two nucleotides of the small RNA molecule and that are not the same as or complementary to the small RNA molecule-binding portion of the forward primer or the small RNA molecule-binding portion of the reverse primer; adjacent to (c) a nucleotide or nucleotide analog that is the same nucleotide base as the 3′-end of the small RNA molecule-binding potion as or is complementary to the 3′-end of the small RNA molecule-binding portion of the reverse primer.

122. The method of claim 121, wherein the reporter probe sequence is not the same as or complementary to either (a) the small RNA molecule-binding portion of the forward primer or (b) the small RNA molecule-binding portion of the reverse primer.

123. The method of claim 112, wherein the small RNA molecule comprises a multiplicity of different small RNA molecules.

124. The method of claim 112, wherein the generating, the detecting, and the quantitating comprise Q-PCR; and the combining further comprises a reporter probe.

125. The method of claim 124, wherein the reporter probe comprises a fluorescent reporter group, a quencher, a minor groove binder, or combinations thereof.

126. The method of claim 125, wherein the reporter probe further comprises: a ribonucleotide, a PNA, an LNA, a 2′-O-alkyl nucleotide, a phosphoroamidate, a FANA, an MP, a CeNA, a tcDNA, or combinations thereof.

127. The method of claim 125, wherein the reporter probe comprises a fluorescent reporter group, at least two deoxyribonucleotides upstream from at least four PNAs, and a quencher.

128. The method of claim 125, wherein the reporter probe further comprises a multiplicity of: ribonucleotides, PNAs, LNAs, 2′-O-alkyl nucleotides, phosphoroamidates, FANAs, MPs, CeNAs, tcDNAs, but not combinations thereof.

129. The method of claim 125, wherein the reporter probe comprises: (a) a nucleotide or nucleotide analog that is the same as the 3′-end of the small RNA molecule-binding portion or is complementary to the 3′-end of the small RNA molecule-binding portion of the forward primer; adjacent to (b) at least two nucleotides or nucleotide analogs that are the same as or are complementary to at least two nucleotides of the small RNA molecule and that are not the same as or complementary to the small RNA molecule-binding portion of the forward primer or the small RNA molecule-binding portion of the reverse primer; adjacent to (c) a nucleotide or nucleotide analog that is the same nucleotide base as the 3′-end of the small RNA molecule-binding potion as or is complementary to the 3′-end of the small RNA molecule-binding portion of the reverse primer.

130. The method of claim 125, wherein the reporter probe sequence is not the same as or complementary to either (a) the small RNA molecule-binding portion of the forward primer or (b) the small RNA molecule-binding portion of the reverse primer.

131. The method of claim 112, wherein a small RNA molecule comprises 17 to 29 ribonucleotides.

132. The method of claim 131, wherein the small RNA molecules comprise a miRNA, a siRNA, or a miRNA and a siRNA.

133. A method for quantitating a small RNA molecule comprising:
combining the small RNA molecule, a first primer set, a second primer set, a reporter probe, a first extending enzyme, and optionally, a second extending enzyme, wherein (a) the first primer set comprises: (1) forward primer comprising (i) a primer-binding portion that is upstream from (ii) a small RNA molecule-binding portion that includes six, seven, eight, or nine nucleotides having the same sequence as a first region of the small RNA molecule and (2) a reverse primer comprising (i) a primer-binding portion that is upstream from (ii) a small RNA molecule-binding portion that includes six, seven, eight, or nine nucleotides that are complementary to a second region of the small RNA molecule; (b) the second primer set comprises a first primer and a second primer, wherein the first primer, the second primer, or the first primer and the second primer comprise a universal priming sequence, a hybridization tag, or a universal priming sequence and a hybridization tag; and (c) the reporter probe comprises a fluorescent reporter group, a quencher, a minor groove binder, or combinations thereof; and wherein the reporter probe sequence is not the same as or complementary to either (a) the small RNA molecule-binding portion of the forward primer or (b) the small RNA molecule-binding portion of the reverse primer;
generating a reverse-transcribed product, a first amplicon, an additional first amplicon, and a second amplicon;
detecting the second amplicons; and
quantitating the small RNA molecule.

134. The method of claim 133, wherein: the small RNA molecule comprises a multiplicity of different small RNA molecules; the first primer set comprises a multiplicity of different first primer sets; the reporter probe comprises a multiplicity of different reporter probes; and the detecting comprises detecting a multiplicity of different second amplicons.

135. The method of claim 133, wherein the small RNA molecule comprises 17 to 29 ribonucleotides.

136. The method of claim 135, wherein the small RNA molecule comprises a miRNA, a siRNA, or a miRNA and a siRNA.

137. A method for quantitating a polynucleotide comprising:
hybridizing a reverse primer of a first primer set to the polynucleotide, wherein the reverse primer comprises: (a) a primer-binding portion that is upstream from (b) a polynucleotide-binding portion comprising no more than ten nucleotides that are complementary to a second region of the polynucleotide;
extending the hybridized reverse primer with a first extending enzyme to generate a first product;
hybridizing a forward primer of the first primer set to the first product, wherein the forward primer comprises: (a) a primer-binding portion that is upstream from (b) a polynucleotide-binding portion comprising no more than ten nucleotides having the same sequence as a first region of the polynucleotide;
extending the hybridized forward primer with a second extending enzyme to generate a first amplicon;
amplifying the first amplicon to generate an additional first amplicon;
combining the additional first amplicon with a second primer set;
amplifying the additional first amplicon to generate a second amplicon;
detecting the second amplicon; and
quantitating the polynucleotide.

138. The method of claim 137, wherein the first extending enzyme and the second extending enzyme are the same enzyme.

139. The method of claim 137, wherein the first extending enzyme and the second extending enzyme are different enzymes.

140. The method of claim 137, wherein the combining further comprises a third extending enzyme.

141. The method of claim 140, wherein (a) the second extending enzyme and the third extending enzyme are the same enzymes and (b) the first extending enzyme and the second extending enzyme are different enzymes.

142. The method of claim 137, wherein the generating the second amplicon, the detecting, and the quantitating comprise a real-time instrument.

143. The method of polynucleotide comprise a miRNA, a siRNA, a miRNA precursor, or combinations thereof.

144. The method of claim 137, wherein the polynucleotide-binding portion of the forward primer comprises six, seven, eight, or nine nucleotides having the same sequence as the first region of the polynucleotide.

145. The method of claim 137, wherein the polynucleotide-binding portion of the reverse primer comprises six, seven, eight, or nine nucleotides that are complementary to the second region of the polynucleotide.

146. The method of claim 145, wherein the polynucleotide-binding portion of the forward primer comprises six, seven, eight, or nine nucleotides having the same sequence as the first region of the polynucleotide.

147. The method of claim 137, wherein a primer of the second primer set comprises a universal priming sequence, a hybridization tag, or a universal priming sequence and a hybridization tag.

148. The method of claim 147, wherein both the first primer and the second primer of the second primer set comprise a universal priming sequence, a hybridization tag, or a universal priming sequence and a hybridization tag, wherein the universal priming sequences are the same or different and the hybridization tags are the same or different.

149. The method of claim 137, wherein a second amplicon comprises an affinity tag, a reporter group, a mobility modifier, a hybridization tag, or combinations thereof.

150. The method of claim 137, wherein the detecting comprises a mobility-dependent analytical technique.

151. The method of claim 137, wherein the detecting further comprises a reporter probe.

152. The method of claim 151, wherein the reporter probe comprises a fluorescent reporter group, a quencher, a minor groove binder, or combinations thereof.

153. The method of claim 152, wherein the reporter probe comprises: (a) a nucleotide or nucleotide analog that has the same nucleotide base as the 3′-end of the polynucleotide-binding portion of the forward primer or is complementary to the 3′-end of the polynucleotide-binding portion of the forward primer; adjacent to (b) at least two nucleotides or nucleotide analogs that have the same nucleotide bases as or are complementary to at least two nucleotides of the polynucleotide and that are not the same as or complementary to the polynucleotide-binding portion of the forward primer or the polynucleotide-binding portion of the reverse primer; adjacent to (c) a nucleotide or nucleotide analog that has the same nucleotide base as the 3′-end of the polynucleotide-binding portion of the reverse primer or is complementary to the 3′-end of the polynucleotide-binding portion of the reverse primer.

154. The method of claim 152, wherein the reporter probe sequence is not the same as or complementary to either (a) the polynucleotide-binding portion of the forward primer or (b) the polynucleotide-binding portion of the reverse primer.

155. The method of claim 137, wherein: the polynucleotide comprises a multiplicity of different polynucleotides; the first primer set comprises a multiplicity of different first primer sets; and the detecting comprises detecting a multiplicity of different second amplicons.

156. The method of claim 137, wherein the polynucleotide comprises no more than 100 nucleotides.

157. The method of claim 156, wherein the polynucleotide comprises a miRNA precursor and wherein the first product comprises a reverse-transcribed product.

158. The method of claim 156, wherein the polynucleotide comprises a deoxyribonucleotide.

159. The method of claim 137, wherein the polynucleotide comprises a multiplicity of different polynucleotides.

160. The method of claim 137, wherein the amplifying, the detecting, and the quantitating comprise Q-PCR.

161. The method of claim 160, wherein: (a) the polynucleotide comprises a multiplicity of different polynucleotides; (b) the first primer set comprises a multiplicity of different first primer sets; (c) the detecting comprises a multiplicity of different second amplicons; and (d) the Q-PCR comprises performing a multiplicity of different assays, wherein an assay comprises (i) a second primer set, (ii) a reporter probe, (iii) and an aliquot of the additional first amplicons; and wherein at least one of the multiplicity of different assays is designed to quantitate a subset of the multiplicity of different polynucleotides.

162. The method of claim 161, wherein the reporter probe comprises a fluorescent reporter group, a quencher, a minor groove binder, or combinations thereof.

163. The method of claim 162, wherein the reporter probe comprises: (a) a nucleotide or nucleotide analog that has the same nucleotide base as the polynucleotide-binding portion of the forward primer or is complementary to the polynucleotide-binding portion of the forward primer; adjacent to (b) at least two nucleotides or nucleotide analogs that have the same nucleotide bases as or are complementary to at least two nucleotides of the polynucleotide and that are not the same as or complementary to the polynucleotide-binding portion of the forward primer or the polynucleotide-binding portion of the reverse primer; adjacent to (c) a nucleotide or nucleotide analog that has the same nucleotide base as the polynucleotide-binding portion of the reverse primer or is complementary to the polynucleotide-binding portion of the reverse primer.

164. The method of claim 162, wherein the reporter probe sequence is not the same as or complementary to either (a) the polynucleotide-binding portion of the forward primer or (b) the polynucleotide-binding portion of the reverse primer.

165. The method of claim 160, wherein the reporter probe further comprises: a deoxyribonucleotide, a ribonucleotide, a PNA, a LNA, a 2′-O-alkyl nucleotide, a phosphoroamidate, a FANA, a MP, a CeNA, a tcDNA, or combinations thereof.

166. The method of claim 165, wherein the reporter probe comprises a fluorescent reporter group, at least two deoxyribonucleotides upstream from at least four PNAs, and a quencher.

167. The method of claim 166, wherein the reporter probe further comprises a minor groove binder.

168. The method of claim 160, wherein the reporter probe further comprises a multiplicity of: deoxyribonucleotides, ribonucleotides, PNAs, LNAs, 2′-O-alkyl nucleotides, phosphoroamidates, FANAs, MPs, CeNAs, tcDNAs, but not combinations thereof.

169. A method for identifying a polynucleotide comprising:
combining the polynucleotide with a first primer set, a second primer set, a first extending enzyme, and optionally, a second extending enzyme, wherein: (a) the first primer set comprises: (1) a forward primer comprising (i) a primer-binding portion that is upstream from (ii) a polynucleotide-binding portion comprising no more than ten nucleotides having the same sequence as a first region of the polynucleotide and (2) a reverse primer comprising (i) a primer-binding portion that is upstream from (ii) a polynucleotide-binding portion comprising no more than ten nucleotides that are complementary to a second region of the polynucleotide; and (b) the second primer set comprises a primer that comprises a universal priming sequence, a hybridization tag, or a universal priming sequence and a hybridization tag;
generating a first amplicon, an additional first amplicon, and a second amplicon;
detecting the second amplicons; and
identifying the polynucleotide.

170. The method of claim 169, wherein the polynucleotide-binding portion of the forward primer comprises six, seven, eight, or nine nucleotides having the same sequence as the first region of the polynucleotide.

171. The method of claim 169, wherein the polynucleotide-binding portion of the reverse primer comprises six, seven, eight, or nine nucleotides that are complementary to the second region of the polynucleotide.

172. The method of claim 171, wherein the polynucleotide-binding portion of the forward primer comprises six, seven, eight, or nine nucleotides having the same sequence as the first region of the polynucleotide.

173. The method of claim 169, wherein both the first primer and the second primer of the second primer set comprise a universal priming sequence, a hybridization tag, or a universal priming sequence and a hybridization tag, wherein the universal priming sequences are the same or different and the hybridization tags are the same or different.

174. The method of claim 169, wherein a second amplicon comprises an affinity tag, a reporter group, a mobility modifier, a hybridization tag, or combinations thereof.

175. The method of claim 169, wherein the detecting comprises a mobility-dependent analytical technique.

176. The method of claim 169, wherein the detecting further comprises a reporter probe.

177. The method of claim 176, wherein the reporter probe comprises a fluorescent reporter group, a quencher, a minor groove binder, or combinations thereof.

178. The method of claim 163, wherein the reporter probe comprises: (a) a nucleotide or nucleotide analog that has the same nucleotide base as the 3′-end of the polynucleotide-binding portion of the forward primer or is complementary to the 3′-end of the polynucleotide-binding portion of the forward primer; adjacent to (b) at least two nucleotides or nucleotide analogs that have the same nucleotide bases as or are complementary to at least two nucleotides of the polynucleotide and that are not the same as or complementary to the polynucleotide-binding portion of the forward primer or the polynucleotide-binding portion of the reverse primer; adjacent to (c) a nucleotide or nucleotide analog that has the same nucleotide base as the 3′-end of the polynucleotide-binding portion of the reverse primer or is complementary to the 3′-end of the polynucleotide-binding portion of the reverse primer.

179. The method of claim 163, wherein the reporter probe sequence is not the same as or complementary to either (a) the polynucleotide-binding portion of the forward primer or (b) the polynucleotide-binding portion of the reverse primer.

180. The method of claim 176, wherein the reporter probe further comprises: a deoxyribonucleotide, a ribonucleotide, a PNA, an LNA, a 2′-O-alkyl nucleotide, a phosphoroamidate, a FANA, an MP, a CeNA, a tcDNA, or combinations thereof.

181. The method of claim 180, wherein the reporter probe comprises a fluorescent reporter group, at least two deoxyribonucleotides upstream from at least four PNAs, and a quencher.

182. The method of claim 176, wherein the reporter probe further comprises a multiplicity of: ribonucleotides, PNAs, LNAs, 2′-O-alkyl nucleotides, phosphoroamidates, FANAs, MPs, CeNAs, tcDNAs, but not combinations thereof.

183. The method of claim 169, wherein the polynucleotide comprises a multiplicity of different polynucleotides.

184. The method of claim 169, wherein the polynucleotide comprises no more than 100 nucleotides.

185. The method of claim 169, wherein the polynucleotide comprises a miRNA precursor.

186. The method of claim 169, wherein the polynucleotide comprises a deoxyribonucleotide.

187. A method for identifying a small RNA molecule comprising:
hybridizing a reverse primer of a first primer set to the small RNA molecule, wherein the reverse primer comprises a small RNA molecule-binding portion comprising no more than ten nucleotides that are complementary to a second region of the small RNA molecule;
extending the hybridized reverse primer with a first extending enzyme to generate a reverse-transcribed product;
hybridizing a forward primer of the first primer set to the reverse-transcribed product, wherein the forward primer comprises a small RNA molecule-binding portion comprising no more than ten nucleotides that are the same as a first region of the small RNA molecule;
extending the hybridized forward primer with a second extending enzyme to generate a first amplicon;
detecting the first amplicon; and
identifying the small RNA molecule.

188. The method of claim 187, wherein the first extending enzyme and the second extending enzyme are the same enzyme or different enzymes.

189. The method of claim 187, wherein the small RNA molecule-binding portion of the forward primer comprises six, seven, eight, or nine nucleotides having the same sequence as the first region of the small RNA molecule.

190. The method of claim 187, wherein the small RNA molecule-binding portion of the reverse primer comprises six, seven, eight, or nine nucleotides that are complementary to the second region of the small RNA molecule.

191. The method of claim 190, wherein the small RNA molecule-binding portion of the forward primer comprises six, seven, eight, or nine nucleotides having the same sequence as the first region of the small RNA molecule.

192. The method of claim 187, wherein the detecting comprises a reporter probe, an intercalating agent, or a reporter probe and an intercalating agent.

193. The method of claim 187, wherein the detecting comprises a real-time detection technique.

194. The method of claim 187, wherein the detecting comprises an end-point detection technique.

195. The method of claim 187, wherein the small RNA molecule comprises a siRNA, a miRNA, or a siRNA and a miRNA

196. A method for quantitating a small RNA molecule comprising:
hybridizing a reverse primer of a first primer set to the small RNA molecule, wherein the reverse primer comprises a small RNA molecule-binding portion comprising no more than ten nucleotides that are complementary to a second region of the small RNA molecule;
extending the hybridized reverse primer with a first extending enzyme to generate a reverse-transcribed product;
hybridizing a forward primer of the first primer set to the reverse-transcribed product, wherein the forward primer comprises a small RNA molecule-binding portion comprising no more than ten nucleotides that are the same as a first region of the small RNA molecule;
extending the hybridized forward primer with a second extending enzyme to generate a first amplicon;
detecting the first amplicon; and
quantitating the small RNA molecule.

197. The method of claim 196, wherein the first extending enzyme and the second extending enzyme are the same enzyme or different enzymes.

198. The method of claim 196, wherein the small RNA molecule-binding portion of the forward primer comprises six, seven, eight, or nine nucleotides having the same sequence as the first region of the small RNA molecule.

199. The method of claim 196, wherein the small RNA molecule-binding portion of the reverse primer comprises six, seven, eight, or nine nucleotides that are complementary to the second region of the small RNA molecule.

200. The method of claim 199, wherein the small RNA molecule-binding portion of the forward primer comprises six, seven, eight, or nine nucleotides having the same sequence as the first region of the small RNA molecule.

201. The method of claim 196, wherein the detecting comprises a reporter probe, an intercalating agent, or a reporter probe and an intercalating agent.

202. The method of claim 201, wherein the detecting comprises a real-time detection technique.

203. The method of claim 196, wherein the detecting comprises an end-point detection technique.

204. The method of claim 196, wherein the small RNA molecule comprises a siRNA, a miRNA, or a siRNA and a miRNA.

205. The method of claim 196, further comprising amplifying the first amplicon to generate an additional first amplicon; and wherein the detecting further comprises detecting the additional first amplicon.

206. The method of claim 205, wherein the detecting comprises a reporter probe, an intercalating agent, or a reporter probe and an intercalating agent.

207. A method for identifying a polynucleotide comprising:
hybridizing a reverse primer of a first primer set to the polynucleotide, wherein the reverse primer comprises: (a) a primer-binding portion that is upstream from (b) a polynucleotide-binding portion comprising no more than ten nucleotides that are complementary to a second region of the polynucleotide;
extending the hybridized reverse primer with a first extending enzyme to generate a first product;
hybridizing a forward primer of the first primer set to the first product, wherein the forward primer comprises: (a) a primer-binding portion that is upstream from (b) a polynucleotide-binding portion comprising no more than ten nucleotides that are the same as a first region of the polynucleotide;
extending the hybridized forward primer with a second extending enzyme to generate a first amplicon;
detecting the first amplicon; and
identifying the polynucleotide.

208. The method of claim 207, wherein the first extending enzyme and the second extending enzyme are the same enzyme or different enzymes.

209. The method of claim 207, wherein the polynucleotide-binding portion of the forward primer comprises six, seven, eight, or nine nucleotides having the same sequence as the first region of the polynucleotide.

210. The method of claim 207, wherein the polynucleotide-binding portion of the reverse primer comprises six, seven, eight, or nine nucleotides that are complementary to the second region of the polynucleotide.

211. The method of claim 210, wherein the polynucleotide-binding portion of the forward primer comprises six, seven, eight, or nine nucleotides having the same sequence as the first region of the polynucleotide.

212. The method of claim 207, wherein the detecting comprises a reporter probe, an intercalating agent, or a reporter probe and an intercalating agent.

213. The method of claim 212, wherein the detecting comprises a real-time detection technique.

214. The method of claim 207, wherein the detecting comprises an end-point detection technique.

215. The method of claim 207, wherein the polynucleotide comprises a deoxyribonucleotide.

216. The method of claim 207, further comprising amplifying the first amplicon to generate an additional first amplicon; and wherein the detecting further comprises detecting the additional first amplicon.

217. The method of claim 216, wherein the detecting comprises a reporter probe, an intercalating agent, or a reporter probe and an intercalating agent.

218. A method for quantitating a polynucleotide comprising:
hybridizing a reverse primer of a first primer set to the polynucleotide, wherein the reverse primer comprises: (a) a primer-binding portion that is upstream from (b) a polynucleotide-binding portion comprising no more than ten nucleotides that are complementary to a second region of the polynucleotide;
extending the hybridized reverse primer with a first extending enzyme to generate a first product;
hybridizing a forward primer of the first primer set to the first product, wherein the forward primer comprises: (a) a primer-binding portion that is upstream from (b) a polynucleotide-binding portion comprising no more than ten nucleotides that are the same as a first region of the polynucleotide;
extending the hybridized forward primer with a second extending enzyme to generate a first amplicon;
detecting the first amplicon; and
quantitating the polynucleotide.

219. The method of claim 218, wherein the first extending enzyme and the second extending enzyme are the same enzyme or different enzymes.

220. The method of claim 218, wherein the polynucleotide-binding portion of the forward primer comprises six, seven, eight, or nine nucleotides having the same sequence as the first region of the polynucleotide.

221. The method of claim 218, wherein the polynucleotide-binding portion of the reverse primer comprises six, seven, eight, or nine nucleotides that are complementary to the second region of the polynucleotide.

222. The method of claim 221, wherein the polynucleotide-binding portion of the forward primer comprises six, seven, eight, or nine nucleotides having the same sequence as the first region of the polynucleotide.

223. The method of claim 218, wherein the detecting comprises a reporter probe, an intercalating agent, or a reporter probe and an intercalating agent.

224. The method of claim 223, wherein the detecting comprises a real-time detection technique.

225. The method of claim 218, wherein the detecting comprises an end-point detection technique.

226. The method of claim 218, wherein the polynucleotide comprises a deoxyribonucleotide.

227. The method of claim 218, further comprising amplifying the first amplicon to generate an additional first amplicon, using a first primer set; and wherein the detecting further comprises detecting the additional first amplicons.

228. The method of claim 227, wherein the detecting comprises a reporter probe, an intercalating agent, or a reporter probe and an intercalating agent.

229. A method for identifying a small RNA molecule comprising:
a step for generating a first product;
a step for generating a first amplicon;
a step for generating an additional first amplicon;
a step for generating a second amplicon;
a step for detecting the second amplicon; and
a step for identifying the small RNA molecule.

230. The method of claim 229, wherein the small RNA molecule comprises a miRNA.

231. The method of claim 229, wherein the step for generating a first product comprises hybridizing a reverse primer of a first primer set with the small RNA molecule and extending the hybridized primer using a first extending enzyme.

232. The method of claim 229, wherein the step for generating a first amplicon comprises hybridizing a forward primer of a corresponding first primer set to the first product and extending the hybridized primer using a second extending enzyme.

233. The method of claim 229, wherein the step for generating a second amplicon comprises hybridizing the first and second primers of the second primer set to the primer-binding portions of the separated strands of the additional first amplicon and extending the hybridized primers using a third extending enzyme.

234. A method for quantitating a small RNA molecule comprising:
a step for generating a first product;
a step for generating a first amplicon;
a step for generating an additional first amplicon;
a step for generating a second amplicon;
a step for detecting the second amplicon; and
a step for quantitating the small RNA molecule.

235. The method of claim 234, wherein the small RNA molecule comprises a miRNA.

236. The method of claim 234, wherein the step for generating a first product comprises hybridizing a reverse primer of a first primer set with the small RNA molecule and extending the hybridized primer using a first extending enzyme.

237. The method of claim 234, wherein the step for generating a first amplicon comprises hybridizing a forward primer of a corresponding first primer set to the first product and extending the hybridized primer using a second extending enzyme.

238. The method of claim 234, wherein the step for generating a second amplicon comprises hybridizing the first and second primers of the second primer set to the primer-binding portions of the separated strands of the additional first amplicon and extending the hybridized primers using a third extending enzyme.

239. A kit comprising the primer of claim 1.

240. The kit of claim 239, wherein the target-binding portion of the primer comprises six, seven, eight, or nine nucleotides.

241. A kit comprising the primer of claim 6.

242. The kit of claim 241, wherein the target-binding portion of the primer comprises six, seven, eight, or nine nucleotides.

243. A kit comprising the primer set of claim 11.

244. The kit of claim 243, further comprising the reporter probe of claim 15.

245. The kit of claim 243, wherein the target-binding portion of the forward primer comprises six, seven, eight, or nine nucleotides.

246. The kit of claim 243, wherein the target-binding portion of the reverse primer comprises six, seven, eight, or nine nucleotides.

247. The kit of claim 246, wherein the target-binding portion of the forward primer comprises six, seven, eight, or nine nucleotides.


SEQUENCE LISTING (225 sequences)

SEQ ID NO: 1 - RNA, 22 nt, Artificial Sequence
  Description of Artificial Sequence Synthetic Oligonucleotide
gaagagauacgcccugguuccu

SEQ ID NO: 2 - DNA, 28 bp, Artificial Sequence
  Description of Artificial Sequence Synthetic Oligonucleotide
accgactccagctcccgaacgaagagat

SEQ ID NO: 3 - DNA, 26 bp, Artificial Sequence
  Description of Artificial Sequence Synthetic Oligonucleotide
gtgtcgtggagtcggcaaaggaacca

SEQ ID NO: 4 - DNA, 21 bp, Artificial Sequence
  Description of Artificial Sequence Synthetic Oligonucleotide
aagagatacgccctggttcct

SEQ ID NO: 5 - DNA, 18 bp, Artificial Sequence
  Description of Artificial Sequence Synthetic Oligonucleotide
gtgtcgtggagtcggcaa

SEQ ID NO: 6 - DNA, 20 bp, Artificial Sequence
  Description of Artificial Sequence Synthetic Oligonucleotide
accgactccagctcccgaac

SEQ ID NO: 7 - DNA, 30 bp, Artificial Sequence
  Description of Artificial Sequence Synthetic Oligonucleotide
accgactccagctcccgaacgaagagatac

SEQ ID NO: 8 - DNA, 28 bp, Artificial Sequence
  Description of Artificial Sequence Synthetic Oligonucleotide
gtgtcgtggagtcggcaaaggaaccagg

SEQ ID NO: 9 - DNA, 29 bp, Artificial Sequence
  Description of Artificial Sequence Synthetic Oligonucleotide
accgactccagctcccgaacgaagagata

SEQ ID NO: 10 - DNA, 27 bp, Artificial Sequence
  Description of Artificial Sequence Synthetic Oligonucleotide
gtgtcgtggagtcggcaaaggaaccag

SEQ ID NO: 11 - DNA, 27 bp, Artificial Sequence
  Description of Artificial Sequence Synthetic Oligonucleotide
accgactccagctcccgaacgaagaga

SEQ ID NO: 12 - DNA, 25 bp, Artificial Sequence
  Description of Artificial Sequence Synthetic Oligonucleotide
gtgtcgtggagtcggcaaaggaacc

SEQ ID NO: 13 - DNA, 26 bp, Artificial Sequence
  Description of Artificial Sequence Synthetic Oligonucleotide
accgactccagctcccgaacgaagag

SEQ ID NO: 14 - DNA, 24 bp, Artificial Sequence
  Description of Artificial Sequence Synthetic Oligonucleotide
gtgtcgtggagtcggcaaaggaac

SEQ ID NO: 15 - RNA, 22 nt, Artificial Sequence
  Description of Artificial Sequence Synthetic Oligonucleotide
ugagguaguagguuguauaguu

SEQ ID NO: 16 - DNA, 30 bp, Artificial Sequence
  Description of Artificial Sequence Synthetic Oligonucleotide
accgactccagctcccgaacaatgaggtag

SEQ ID NO: 17 - DNA, 30 bp, Artificial Sequence
  Description of Artificial Sequence Synthetic Oligonucleotide
ttggcctgttcccgctcgctaaaactatac

SEQ ID NO: 18 - RNA, 21 nt, Artificial Sequence
  Description of Artificial Sequence Synthetic Oligonucleotide
ucccugagaccucaaguguga

SEQ ID NO: 19 - DNA, 30 bp, Artificial Sequence
  Description of Artificial Sequence Synthetic Oligonucleotide
accgactccagctcccgaacaatccctgag

SEQ ID NO: 20 - DNA, 30 bp, Artificial Sequence
  Description of Artificial Sequence Synthetic Oligonucleotide
tcgcggacggtgagcactgcaatcacactt

SEQ ID NO: 21 - RNA, 22 nt, Artificial Sequence
  Description of Artificial Sequence Synthetic Oligonucleotide
uaaagugcuuauagugcaggua

SEQ ID NO: 22 - DNA, 30 bp, Artificial Sequence
  Description of Artificial Sequence Synthetic Oligonucleotide
accgactccagctcccgaacaataaagtgc

SEQ ID NO: 23 - DNA, 30 bp, Artificial Sequence
  Description of Artificial Sequence Synthetic Oligonucleotide
ttcccgctggtgccactccgaatacctgca

SEQ ID NO: 24 - RNA, 22 nt, Artificial Sequence
  Description of Artificial Sequence Synthetic Oligonucleotide
cuuucagucggauguuugcagc

SEQ ID NO: 25 - DNA, 30 bp, Artificial Sequence
  Description of Artificial Sequence Synthetic Oligonucleotide
accgactccagctcccgaacaactttcagt

SEQ ID NO: 26 - DNA, 30 bp, Artificial Sequence
  Description of Artificial Sequence Synthetic Oligonucleotide
cgctcgctttggcctgctgcaagctgcaaa

SEQ ID NO: 27 - RNA, 21 nt, Artificial Sequence
  Description of Artificial Sequence Synthetic Oligonucleotide
uggaagacuagugauuuuguu

SEQ ID NO: 28 - DNA, 30 bp, Artificial Sequence
  Description of Artificial Sequence Synthetic Oligonucleotide
accgactccagctcccgaacaatggaagac

SEQ ID NO: 29 - DNA, 30 bp, Artificial Sequence
  Description of Artificial Sequence Synthetic Oligonucleotide
cgctgcgtgagggctcggacaaaacaaaat

SEQ ID NO: 30 - RNA, 23 nt, Artificial Sequence
  Description of Artificial Sequence Synthetic Oligonucleotide
agcagcauuguacagggcuauca

SEQ ID NO: 31 - DNA, 30 bp, Artificial Sequence
  Description of Artificial Sequence Synthetic Oligonucleotide
accgactccagctcccgaacaaagcagcat

SEQ ID NO: 32 - DNA, 30 bp, Artificial Sequence
  Description of Artificial Sequence Synthetic Oligonucleotide
ttccacccgcgtcgtcctgcaatgatagcc

SEQ ID NO: 33 - RNA, 21 nt, Artificial Sequence
  Description of Artificial Sequence Synthetic Oligonucleotide
uuuggauugaagggagcucua

SEQ ID NO: 34 - DNA, 30 bp, Artificial Sequence
  Description of Artificial Sequence Synthetic Oligonucleotide
accgactccagctcccgaacaatttggatt

SEQ ID NO: 35 - DNA, 30 bp, Artificial Sequence
  Description of Artificial Sequence Synthetic Oligonucleotide
agcacgcttccgggacccacaatagagctc

SEQ ID NO: 36 - RNA, 21 nt, Artificial Sequence
  Description of Artificial Sequence Synthetic Oligonucleotide
uugaaagugacuacaucgggg

SEQ ID NO: 37 - DNA, 30 bp, Artificial Sequence
  Description of Artificial Sequence Synthetic Oligonucleotide
accgactccagctcccgaacaattgaaagt

SEQ ID NO: 38 - DNA, 30 bp, Artificial Sequence
  Description of Artificial Sequence Synthetic Oligonucleotide
aggcggtgttggctcgaggcaaccccgatg

SEQ ID NO: 39 - RNA, 23 nt, Artificial Sequence
  Description of Artificial Sequence Synthetic Oligonucleotide
uaaggcacgcggugaaugccaag

SEQ ID NO: 40 - DNA, 30 bp, Artificial Sequence
  Description of Artificial Sequence Synthetic Oligonucleotide
accgactccagctcccgaacaataaggcac

SEQ ID NO: 41 - DNA, 30 bp, Artificial Sequence
  Description of Artificial Sequence Synthetic Oligonucleotide
gaggttcccgcttcgcccacaacttggcat

SEQ ID NO: 42 - RNA, 21 nt, Artificial Sequence
  Description of Artificial Sequence Synthetic Oligonucleotide
uugugcgugugacagcggcua

SEQ ID NO: 43 - DNA, 30 bp, Artificial Sequence
  Description of Artificial Sequence Synthetic Oligonucleotide
accgactccagctcccgaacaattgtgcgt

SEQ ID NO: 44 - DNA, 30 bp, Artificial Sequence
  Description of Artificial Sequence Synthetic Oligonucleotide
ggtgccacgtgatcgctccgaatagccgct

SEQ ID NO: 45 - RNA, 23 nt, Artificial Sequence
  Description of Artificial Sequence Synthetic Oligonucleotide
uaucacagccagcuuugaugugc

SEQ ID NO: 46 - DNA, 30 bp, Artificial Sequence
  Description of Artificial Sequence Synthetic Oligonucleotide
accgactccagctcccgaacaatatcacag

SEQ ID NO: 47 - DNA, 30 bp, Artificial Sequence
  Description of Artificial Sequence Synthetic Oligonucleotide
ctgcaggcgcgtgagggaggaagcacatca

SEQ ID NO: 48 - RNA, 22 nt, Artificial Sequence
  Description of Artificial Sequence Synthetic Oligonucleotide
uagcagcacguaaauauuggcg

SEQ ID NO: 49 - DNA, 30 bp, Artificial Sequence
  Description of Artificial Sequence Synthetic Oligonucleotide
accgactccagctcccgaacaatagcagca

SEQ ID NO: 50 - DNA, 30 bp, Artificial Sequence
  Description of Artificial Sequence Synthetic Oligonucleotide
gccatccgcgctcagtccacaacgccaata

SEQ ID NO: 51 - RNA, 22 nt, Artificial Sequence
  Description of Artificial Sequence Synthetic Oligonucleotide
uagcuuaucagacugauguuga

SEQ ID NO: 52 - DNA, 30 bp, Artificial Sequence
  Description of Artificial Sequence Synthetic Oligonucleotide
accgactccagctcccgaacaatagcttat

SEQ ID NO: 53 - DNA, 30 bp, Artificial Sequence
  Description of Artificial Sequence Synthetic Oligonucleotide
ggtgggacgcgtctgcctcgaatcaacatc

SEQ ID NO: 54 - RNA, 22 nt, Artificial Sequence
  Description of Artificial Sequence Synthetic Oligonucleotide
aagcugccaguugaagaacugu

SEQ ID NO: 55 - DNA, 30 bp, Artificial Sequence
  Description of Artificial Sequence Synthetic Oligonucleotide
accgactccagctcccgaacaaaagctgcc

SEQ ID NO: 56 - DNA, 30 bp, Artificial Sequence
  Description of Artificial Sequence Synthetic Oligonucleotide
tcgcttgggtcttcgcaggcaaacagttct

SEQ ID NO: 57 - RNA, 22 nt, Artificial Sequence
  Description of Artificial Sequence Synthetic Oligonucleotide
uucaaguaauccaggauaggcu

SEQ ID NO: 58 - DNA, 30 bp, Artificial Sequence
  Description of Artificial Sequence Synthetic Oligonucleotide
accgactccagctcccgaacaattcaagta

SEQ ID NO: 59 - DNA, 30 bp, Artificial Sequence
  Description of Artificial Sequence Synthetic Oligonucleotide
ttgggaccctcgggactcgcaaagcctatc

SEQ ID NO: 60 - RNA, 22 nt, Artificial Sequence
  Description of Artificial Sequence Synthetic Oligonucleotide
cuagcaccaucugaaaucgguu

SEQ ID NO: 61 - DNA, 30 bp, Artificial Sequence
  Description of Artificial Sequence Synthetic Oligonucleotide
accgactccagctcccgaacaactagcacc

SEQ ID NO: 62 - DNA, 30 bp, Artificial Sequence
  Description of Artificial Sequence Synthetic Oligonucleotide
tccggaccagcactgcaggcaaaaccgatt

SEQ ID NO: 63 - RNA, 22 nt, Artificial Sequence
  Description of Artificial Sequence Synthetic Oligonucleotide
uggcagugucuuagcugguugu

SEQ ID NO: 64 - DNA, 30 bp, Artificial Sequence
  Description of Artificial Sequence Synthetic Oligonucleotide
accgactccagctcccgaacaatggcagtg

SEQ ID NO: 65 - DNA, 30 bp, Artificial Sequence
  Description of Artificial Sequence Synthetic Oligonucleotide
tcgcgagggtcttccggaggaaacaaccag

SEQ ID NO: 66 - RNA, 23 nt, Artificial Sequence
  Description of Artificial Sequence Synthetic Oligonucleotide
aacauucaacgcugucggugagu

SEQ ID NO: 67 - DNA, 30 bp, Artificial Sequence
  Description of Artificial Sequence Synthetic Oligonucleotide
accgactccagctcccgaacaaaacattca

SEQ ID NO: 68 - DNA, 30 bp, Artificial Sequence
  Description of Artificial Sequence Synthetic Oligonucleotide
tggtccaccctgctgctcgcaaactcaccg

SEQ ID NO: 69 - RNA, 24 nt, Artificial Sequence
  Description of Artificial Sequence Synthetic Oligonucleotide
cucuaauacugccugguaaugaug

SEQ ID NO: 70 - DNA, 30 bp, Artificial Sequence
  Description of Artificial Sequence Synthetic Oligonucleotide
accgactccagctcccgaacaactctaata

SEQ ID NO: 71 - DNA, 30 bp, Artificial Sequence
  Description of Artificial Sequence Synthetic Oligonucleotide
tcgcgaccgtctgctcaggcaacatcatta

SEQ ID NO: 72 - RNA, 21 nt, Artificial Sequence
  Description of Artificial Sequence Synthetic Oligonucleotide
ugucaguuugucaaauacccc

SEQ ID NO: 73 - DNA, 30 bp, Artificial Sequence
  Description of Artificial Sequence Synthetic Oligonucleotide
accgactccagctcccgaacaatgtcagtt

SEQ ID NO: 74 - DNA, 30 bp, Artificial Sequence
  Description of Artificial Sequence Synthetic Oligonucleotide
cgctggaccgctaggcacccaaggggtatt

SEQ ID NO: 75 - RNA, 23 nt, Artificial Sequence
  Description of Artificial Sequence Synthetic Oligonucleotide
caagucacuagugguuccguuua

SEQ ID NO: 76 - DNA, 30 bp, Artificial Sequence
  Description of Artificial Sequence Synthetic Oligonucleotide
accgactccagctcccgaacaacaagtcac

SEQ ID NO: 77 - DNA, 30 bp, Artificial Sequence
  Description of Artificial Sequence Synthetic Oligonucleotide
ttgggcgttggtgcgtcctgaataaacgga

SEQ ID NO: 78 - RNA, 22 nt, Artificial Sequence
  Description of Artificial Sequence Synthetic Oligonucleotide
gcacauuacacggucgaccucu

SEQ ID NO: 79 - DNA, 30 bp, Artificial Sequence
  Description of Artificial Sequence Synthetic Oligonucleotide
accgactccagctcccgaacaagcacatta

SEQ ID NO: 80 - DNA, 30 bp, Artificial Sequence
  Description of Artificial Sequence Synthetic Oligonucleotide
gctccagttccgggtgcgctaaagaggtcg

SEQ ID NO: 81 - RNA, 23 nt, Artificial Sequence
  Description of Artificial Sequence Synthetic Oligonucleotide
cgcauccccuagggcauuggugu

SEQ ID NO: 82 - DNA, 30 bp, Artificial Sequence
  Description of Artificial Sequence Synthetic Oligonucleotide
accgactccagctcccgaacaacgcatccc

SEQ ID NO: 83 - DNA, 30 bp, Artificial Sequence
  Description of Artificial Sequence Synthetic Oligonucleotide
gcgtgaggttgggagggcgtaaacaccaat

SEQ ID NO: 84 - RNA, 22 nt, Artificial Sequence
  Description of Artificial Sequence Synthetic Oligonucleotide
cuggcccucucugcccuuccgu

SEQ ID NO: 85 - DNA, 30 bp, Artificial Sequence
  Description of Artificial Sequence Synthetic Oligonucleotide
accgactccagctcccgaacaactggccct

SEQ ID NO: 86 - DNA, 30 bp, Artificial Sequence
  Description of Artificial Sequence Synthetic Oligonucleotide
ttggttgggaggctgcggtgaaacggaagg

SEQ ID NO: 87 - RNA, 23 nt, Artificial Sequence
  Description of Artificial Sequence Synthetic Oligonucleotide
uacccuguagauccgaauuugug

SEQ ID NO: 88 - DNA, 30 bp, Artificial Sequence
  Description of Artificial Sequence Synthetic Oligonucleotide
accgactccagctcccgaacaataccctgt

SEQ ID NO: 89 - DNA, 30 bp, Artificial Sequence
  Description of Artificial Sequence Synthetic Oligonucleotide
cgtcgctctggttcgctcgcaacacaaatt

SEQ ID NO: 90 - RNA, 22 nt, Artificial Sequence
  Description of Artificial Sequence Synthetic Oligonucleotide
uacccuguagaaccgaauuugu

SEQ ID NO: 91 - DNA, 30 bp, Artificial Sequence; identical to SEQ ID NO: 88
  Description of Artificial Sequence Synthetic Oligonucleotide

SEQ ID NO: 92 - DNA, 30 bp, Artificial Sequence
  Description of Artificial Sequence Synthetic Oligonucleotide
ggtgacccgtgacgctgccaaaacaaattc

SEQ ID NO: 93 - RNA, 21 nt, Artificial Sequence
  Description of Artificial Sequence Synthetic Oligonucleotide
aucacauugccagggauuucc

SEQ ID NO: 94 - DNA, 30 bp, Artificial Sequence
  Description of Artificial Sequence Synthetic Oligonucleotide
accgactccagctcccgaacaaatcacatt

SEQ ID NO: 95 - DNA, 30 bp, Artificial Sequence
  Description of Artificial Sequence Synthetic Oligonucleotide
aggcttggcgctcgtcacccaaggaaatcc

SEQ ID NO: 96 - RNA, 22 nt, Artificial Sequence
  Description of Artificial Sequence Synthetic Oligonucleotide
uucacaguggcuaaguuccgcc

SEQ ID NO: 97 - DNA, 30 bp, Artificial Sequence
  Description of Artificial Sequence Synthetic Oligonucleotide
accgactccagctcccgaacaattcacagt

SEQ ID NO: 98 - DNA, 30 bp, Artificial Sequence
  Description of Artificial Sequence Synthetic Oligonucleotide
gtctccaccgctgccatcgcaaggcggaac

SEQ ID NO: 99 - RNA, 23 nt, Artificial Sequence
  Description of Artificial Sequence Synthetic Oligonucleotide
uguaaacauccuacacucucagc

SEQ ID NO: 100 - DNA, 30 bp, Artificial Sequence
  Description of Artificial Sequence Synthetic Oligonucleotide
accgactccagctcccgaacaatgtaaaca

SEQ ID NO: 101 - DNA, 30 bp, Artificial Sequence
  Description of Artificial Sequence Synthetic Oligonucleotide
cgtcgtgagccatcgcctgcaagctgagag

SEQ ID NO: 102 - RNA, 22 nt, Artificial Sequence
  Description of Artificial Sequence Synthetic Oligonucleotide
ugagaugaagcacuguagcuca

SEQ ID NO: 103 - DNA, 30 bp, Artificial Sequence
  Description of Artificial Sequence Synthetic Oligonucleotide
accgactccagctcccgaacaatgagatga

SEQ ID NO: 104 - DNA, 30 bp, Artificial Sequence
  Description of Artificial Sequence Synthetic Oligonucleotide
ggacgaggaggccgcttggtaatgagctac

SEQ ID NO: 105 - RNA, 24 nt, Artificial Sequence
  Description of Artificial Sequence Synthetic Oligonucleotide
guccaguuuucccaggaaucccuu

SEQ ID NO: 106 - DNA, 30 bp, Artificial Sequence
  Description of Artificial Sequence Synthetic Oligonucleotide
accgactccagctcccgaacaagtccagtt

SEQ ID NO: 107 - DNA, 30 bp, Artificial Sequence
  Description of Artificial Sequence Synthetic Oligonucleotide
cctgctcgtcgctccgttggaaaagggatt

SEQ ID NO: 108 - RNA, 21 nt, Artificial Sequence
  Description of Artificial Sequence Synthetic Oligonucleotide
uagguaguuucauguuguugg

SEQ ID NO: 109 - DNA, 30 bp, Artificial Sequence
  Description of Artificial Sequence Synthetic Oligonucleotide
accgactccagctcccgaacaataggtagt

SEQ ID NO: 110 - DNA, 30 bp, Artificial Sequence
  Description of Artificial Sequence Synthetic Oligonucleotide
gtgatcgccgtccctgtccgaaccaacaac

SEQ ID NO: 111 - RNA, 21 nt, Artificial Sequence
  Description of Artificial Sequence Synthetic Oligonucleotide
uaaucucagcuggcaacugug

SEQ ID NO: 112 - DNA, 30 bp, Artificial Sequence
  Description of Artificial Sequence Synthetic Oligonucleotide
accgactccagctcccgaacaataatctca

SEQ ID NO: 113 - DNA, 30 bp, Artificial Sequence
  Description of Artificial Sequence Synthetic Oligonucleotide
ttccgaggcctgacccgaccaacacagttg

SEQ ID NO: 114 - DNA, 24 bp, Artificial Sequence
  Description of Artificial Sequence Synthetic Oligonucleotide
aatgaggtagtaggttgtatagtt

SEQ ID NO: 115 - DNA, 12 bp, Artificial Sequence
  Description of Artificial Sequence Synthetic Oligonucleotide
aggtagtaggtt

SEQ ID NO: 116 - DNA, 23 bp, Artificial Sequence
  Description of Artificial Sequence Synthetic Oligonucleotide
aatccctgagacctcaagtgtga

SEQ ID NO: 117 - DNA, 13 bp, Artificial Sequence
  Description of Artificial Sequence Synthetic Oligonucleotide
tccctgagacctc

SEQ ID NO: 118 - DNA, 24 bp, Artificial Sequence
  Description of Artificial Sequence Synthetic Oligonucleotide
aataaagtgcttatagtgcaggta

SEQ ID NO: 119 - DNA, 16 bp, Artificial Sequence
  Description of Artificial Sequence Synthetic Oligonucleotide
taaagtgcttatagtg

SEQ ID NO: 120 - DNA, 24 bp, Artificial Sequence
  Description of Artificial Sequence Synthetic Oligonucleotide
aactttcagtcggatgtttgcagc

SEQ ID NO: 121 - DNA, 14 bp, Artificial Sequence
  Description of Artificial Sequence Synthetic Oligonucleotide
ctttcagtcggatg

SEQ ID NO: 122 - DNA, 23 bp, Artificial Sequence
  Description of Artificial Sequence Synthetic Oligonucleotide
aatggaagactagtgattttgtt

SEQ ID NO: 123 - DNA, 12 bp, Artificial Sequence
  Description of Artificial Sequence Synthetic Oligonucleotide
ggaagactagtg

SEQ ID NO: 124 - DNA, 25 bp, Artificial Sequence
  Description of Artificial Sequence Synthetic Oligonucleotide
aaagcagcattgtacagggctatca

SEQ ID NO: 125 - DNA, 13 bp, Artificial Sequence
  Description of Artificial Sequence Synthetic Oligonucleotide
cagcattgtacag

SEQ ID NO: 126 - DNA, 23 bp, Artificial Sequence
  Description of Artificial Sequence Synthetic Oligonucleotide
aatttggattgaagggagctcta

SEQ ID NO: 127 - DNA, 13 bp, Artificial Sequence
  Description of Artificial Sequence Synthetic Oligonucleotide
tttggattgaagg

SEQ ID NO: 128 - DNA, 23 bp, Artificial Sequence
  Description of Artificial Sequence Synthetic Oligonucleotide
aattgaaagtgactacatcgggg

SEQ ID NO: 129 - DNA, 15 bp, Artificial Sequence
  Description of Artificial Sequence Synthetic Oligonucleotide
ttgaaagtgactaca

SEQ ID NO: 130 - DNA, 25 bp, Artificial Sequence
  Description of Artificial Sequence Synthetic Oligonucleotide
aataaggcacgcggtgaatgccaag

SEQ ID NO: 131 - DNA, 10 bp, Artificial Sequence
  Description of Artificial Sequence Synthetic Oligonucleotide
cacgcggtga

SEQ ID NO: 132 - DNA, 23 bp, Artificial Sequence
  Description of Artificial Sequence Synthetic Oligonucleotide
aattgtgcgtgtgacagcggcta

SEQ ID NO: 133 - DNA, 13 bp, Artificial Sequence
  Description of Artificial Sequence Synthetic Oligonucleotide
ttgtgcgtgtgac

SEQ ID NO: 134 - DNA, 25 bp, Artificial Sequence
  Description of Artificial Sequence Synthetic Oligonucleotide
aatatcacagccagctttgatgtgc

SEQ ID NO: 135 - DNA, 13 bp, Artificial Sequence
  Description of Artificial Sequence Synthetic Oligonucleotide
tcacagccagctt

SEQ ID NO: 136 - DNA, 24 bp, Artificial Sequence
  Description of Artificial Sequence Synthetic Oligonucleotide
aatagcagcacgtaaatattggcg

SEQ ID NO: 137 - DNA, 13 bp, Artificial Sequence
  Description of Artificial Sequence Synthetic Oligonucleotide
agcagcacgtaaa

SEQ ID NO: 138 - DNA, 24 bp, Artificial Sequence
  Description of Artificial Sequence Synthetic Oligonucleotide
aatagcttatcagactgatgttga

SEQ ID NO: 139 - DNA, 15 bp, Artificial Sequence
  Description of Artificial Sequence Synthetic Oligonucleotide
tagcttatcagactg

SEQ ID NO: 140 - DNA, 24 bp, Artificial Sequence
  Description of Artificial Sequence Synthetic Oligonucleotide
aaaagctgccagttgaagaactgt

SEQ ID NO: 141 - DNA, 13 bp, Artificial Sequence
  Description of Artificial Sequence Synthetic Oligonucleotide
agctgccagttga

SEQ ID NO: 142 - DNA, 24 bp, Artificial Sequence
  Description of Artificial Sequence Synthetic Oligonucleotide
aattcaagtaatccaggataggct

SEQ ID NO: 143 - DNA, 15 bp, Artificial Sequence
  Description of Artificial Sequence Synthetic Oligonucleotide
ttcaagtaatccagg

SEQ ID NO: 144 - DNA, 24 bp, Artificial Sequence
  Description of Artificial Sequence Synthetic Oligonucleotide
aactagcaccatctgaaatcggtt

SEQ ID NO: 145 - DNA, 14 bp, Artificial Sequence
  Description of Artificial Sequence Synthetic Oligonucleotide
ctagcaccatctga

SEQ ID NO: 146 - DNA, 24 bp, Artificial Sequence
  Description of Artificial Sequence Synthetic Oligonucleotide
aatggcagtgtcttagctggttgt

SEQ ID NO: 147 - DNA, 14 bp, Artificial Sequence
  Description of Artificial Sequence Synthetic Oligonucleotide
tggcagtgtcttag

SEQ ID NO: 148 - DNA, 25 bp, Artificial Sequence
  Description of Artificial Sequence Synthetic Oligonucleotide
aaaacattcaacgctgtcggtgagt

SEQ ID NO: 149 - DNA, 16 bp, Artificial Sequence
  Description of Artificial Sequence Synthetic Oligonucleotide
aacattcaacgctgtc

SEQ ID NO: 150 - DNA, 26 bp, Artificial Sequence
  Description of Artificial Sequence Synthetic Oligonucleotide
aactctaatactgcctggtaatgatg

SEQ ID NO: 151 - DNA, 16 bp, Artificial Sequence
  Description of Artificial Sequence Synthetic Oligonucleotide
ctctaatactgcctgg

SEQ ID NO: 152 - DNA, 23 bp, Artificial Sequence
  Description of Artificial Sequence Synthetic Oligonucleotide
aatgtcagtttgtcaaatacccc

SEQ ID NO: 153 - DNA, 17 bp, Artificial Sequence
  Description of Artificial Sequence Synthetic Oligonucleotide
tgtcagtttgtcaaata

SEQ ID NO: 154 - DNA, 25 bp, Artificial Sequence
  Description of Artificial Sequence Synthetic Oligonucleotide
aacaagtcactagtggttccgttta

SEQ ID NO: 155 - DNA, 13 bp, Artificial Sequence
  Description of Artificial Sequence Synthetic Oligonucleotide
agtcactagtggt

SEQ ID NO: 156 - DNA, 24 bp, Artificial Sequence
  Description of Artificial Sequence Synthetic Oligonucleotide
aagcacattacacggtcgacctct

SEQ ID NO: 157 - DNA, 13 bp, Artificial Sequence
  Description of Artificial Sequence Synthetic Oligonucleotide
cacattacacggt

SEQ ID NO: 158 - DNA, 25 bp, Artificial Sequence
  Description of Artificial Sequence Synthetic Oligonucleotide
aacgcatcccctagggcattggtgt

SEQ ID NO: 159 - DNA, 12 bp, Artificial Sequence
  Description of Artificial Sequence Synthetic Oligonucleotide
atcccctagggc

SEQ ID NO: 160 - DNA, 24 bp, Artificial Sequence
  Description of Artificial Sequence Synthetic Oligonucleotide
aactggccctctctgcccttccgt

SEQ ID NO: 161 - DNA, 14 bp, Artificial Sequence
  Description of Artificial Sequence Synthetic Oligonucleotide
ctggccctctctgc

SEQ ID NO: 162 - DNA, 25 bp, Artificial Sequence
  Description of Artificial Sequence Synthetic Oligonucleotide
aataccctgtagatccgaatttgtg

SEQ ID NO: 163 - DNA, 14 bp, Artificial Sequence
  Description of Artificial Sequence Synthetic Oligonucleotide
taccctgtagatcc

SEQ ID NO: 164 - DNA, 14 bp, Artificial Sequence
  Description of Artificial Sequence Synthetic Oligonucleotide
taccctgtagaacc

SEQ ID NO: 165 - DNA, 23 bp, Artificial Sequence
  Description of Artificial Sequence Synthetic Oligonucleotide
aaatcacattgccagggatttcc

SEQ ID NO: 166 - DNA, 14 bp, Artificial Sequence
  Description of Artificial Sequence Synthetic Oligonucleotide
atcacattgccagg

SEQ ID NO: 167 - DNA, 24 bp, Artificial Sequence
  Description of Artificial Sequence Synthetic Oligonucleotide
aattcacagtggctaagttccgcc

SEQ ID NO: 168 - DNA, 14 bp, Artificial Sequence
  Description of Artificial Sequence Synthetic Oligonucleotide
ttcacagtggctaa

SEQ ID NO: 169 - DNA, 25 bp, Artificial Sequence
  Description of Artificial Sequence Synthetic Oligonucleotide
aatgtaaacatcctacactctcagc

SEQ ID NO: 170 - DNA, 17 bp, Artificial Sequence
  Description of Artificial Sequence Synthetic Oligonucleotide
tgtaaacatcctacact

SEQ ID NO: 171 - DNA, 24 bp, Artificial Sequence
  Description of Artificial Sequence Synthetic Oligonucleotide
aatgagatgaagcactgtagctca

SEQ ID NO: 172 - DNA, 12 bp, Artificial Sequence
  Description of Artificial Sequence Synthetic Oligonucleotide
agatgaagcact

SEQ ID NO: 173 - DNA, 26 bp, Artificial Sequence
  Description of Artificial Sequence Synthetic Oligonucleotide
aagtccagttttcccaggaatccctt

SEQ ID NO: 174 - DNA, 14 bp, Artificial Sequence
  Description of Artificial Sequence Synthetic Oligonucleotide
ccagttttcccagg

SEQ ID NO: 175 - DNA, 23 bp, Artificial Sequence
  Description of Artificial Sequence Synthetic Oligonucleotide
aataggtagtttcatgttgttgg

SEQ ID NO: 176 - DNA, 14 bp, Artificial Sequence
  Description of Artificial Sequence Synthetic Oligonucleotide
aggtagtttcatgt

SEQ ID NO: 177 - DNA, 23 bp, Artificial Sequence
  Description of Artificial Sequence Synthetic Oligonucleotide
aataatctcagctggcaactgtg

SEQ ID NO: 178 - DNA, 14 bp, Artificial Sequence
  Description of Artificial Sequence Synthetic Oligonucleotide
taatctcagctggc

SEQ ID NO: 179 - DNA, 20 bp, Artificial Sequence
  Description of Artificial Sequence Synthetic Oligonucleotide
accgactccagctcccgaac

SEQ ID NO: 180 - DNA, 22 bp, Artificial Sequence
  Description of Artificial Sequence Synthetic Oligonucleotide
ttggcctgttcccgctcgctaa

SEQ ID NO: 181 - DNA, 22 bp, Artificial Sequence
  Description of Artificial Sequence Synthetic Oligonucleotide
tcgcggacggtgagcactgcaa

SEQ ID NO: 182 - DNA, 22 bp, Artificial Sequence
  Description of Artificial Sequence Synthetic Oligonucleotide
ttcccgctggtgccactccgaa

SEQ ID NO: 183 - DNA, 22 bp, Artificial Sequence
  Description of Artificial Sequence Synthetic Oligonucleotide
cgctcgctttggcctgctgcaa

SEQ ID NO: 184 - DNA, 22 bp, Artificial Sequence
  Description of Artificial Sequence Synthetic Oligonucleotide
cgctgcgtgagggctcggacaa

SEQ ID NO: 185 - DNA, 22 bp, Artificial Sequence
  Description of Artificial Sequence Synthetic Oligonucleotide
ttccacccgcgtcgtcctgcaa

SEQ ID NO: 186 - DNA, 22 bp, Artificial Sequence
  Description of Artificial Sequence Synthetic Oligonucleotide
agcacgcttccgggacccacaa

SEQ ID NO: 187 - DNA, 22 bp, Artificial Sequence
  Description of Artificial Sequence Synthetic Oligonucleotide
aggcggtgttggctcgaggcaa

SEQ ID NO: 188 - DNA, 22 bp, Artificial Sequence
  Description of Artificial Sequence Synthetic Oligonucleotide
gaggttcccgcttcgcccacaa

SEQ ID NO: 189 - DNA, 22 bp, Artificial Sequence
  Description of Artificial Sequence Synthetic Oligonucleotide
ggtgccacgtgatcgctccgaa

SEQ ID NO: 190 - DNA, 22 bp, Artificial Sequence
  Description of Artificial Sequence Synthetic Oligonucleotide
ctgcaggcgcgtgagggaggaa

SEQ ID NO: 191 - DNA, 22 bp, Artificial Sequence
  Description of Artificial Sequence Synthetic Oligonucleotide
gccatccgcgctcagtccacaa

SEQ ID NO: 192 - DNA, 22 bp, Artificial Sequence
  Description of Artificial Sequence Synthetic Oligonucleotide
ggtgggacgcgtctgcctcgaa

SEQ ID NO: 193 - DNA, 22 bp, Artificial Sequence
  Description of Artificial Sequence Synthetic Oligonucleotide
tcgcttgggtcttcgcaggcaa

SEQ ID NO: 194 - DNA, 22 bp, Artificial Sequence
  Description of Artificial Sequence Synthetic Oligonucleotide
ttgggaccctcgggactcgcaa

SEQ ID NO: 195 - DNA, 22 bp, Artificial Sequence
  Description of Artificial Sequence Synthetic Oligonucleotide
tccggaccagcactgcaggcaa

SEQ ID NO: 196 - DNA, 22 bp, Artificial Sequence
  Description of Artificial Sequence Synthetic Oligonucleotide
tcgcgagggtcttccggaggaa

SEQ ID NO: 197 - DNA, 22 bp, Artificial Sequence
  Description of Artificial Sequence Synthetic Oligonucleotide
tggtccaccctgctgctcgcaa

SEQ ID NO: 198 - DNA, 22 bp, Artificial Sequence
  Description of Artificial Sequence Synthetic Oligonucleotide
tcgcgaccgtctgctcaggcaa

SEQ ID NO: 199 - DNA, 22 bp, Artificial Sequence
  Description of Artificial Sequence Synthetic Oligonucleotide
cgctggaccgctaggcacccaa

SEQ ID NO: 200 - DNA, 22 bp, Artificial Sequence
  Description of Artificial Sequence Synthetic Oligonucleotide
ttgggcgttggtgcgtcctgaa

SEQ ID NO: 201 - DNA, 22 bp, Artificial Sequence
  Description of Artificial Sequence Synthetic Oligonucleotide
gctccagttccgggtgcgctaa

SEQ ID NO: 202 - DNA, 22 bp, Artificial Sequence
  Description of Artificial Sequence Synthetic Oligonucleotide
gcgtgaggttgggagggcgtaa

SEQ ID NO: 203 - DNA, 22 bp, Artificial Sequence
  Description of Artificial Sequence Synthetic Oligonucleotide
ttggttgggaggctgcggtgaa

SEQ ID NO: 204 - DNA, 22 bp, Artificial Sequence
  Description of Artificial Sequence Synthetic Oligonucleotide
cgtcgctctggttcgctcgcaa

SEQ ID NO: 205 - DNA, 22 bp, Artificial Sequence
  Description of Artificial Sequence Synthetic Oligonucleotide
ggtgacccgtgacgctgccaaa

SEQ ID NO: 206 - DNA, 22 bp, Artificial Sequence
  Description of Artificial Sequence Synthetic Oligonucleotide
aggcttggcgctcgtcacccaa

SEQ ID NO: 207 - DNA, 22 bp, Artificial Sequence
  Description of Artificial Sequence Synthetic Oligonucleotide
gtctccaccgctgccatcgcaa

SEQ ID NO: 208 - DNA, 22 bp, Artificial Sequence
  Description of Artificial Sequence Synthetic Oligonucleotide
cgtcgtgagccatcgcctgcaa

SEQ ID NO: 209 - DNA, 22 bp, Artificial Sequence
  Description of Artificial Sequence Synthetic Oligonucleotide
ggacgaggaggccgcttggtaa

SEQ ID NO: 210 - DNA, 22 bp, Artificial Sequence
  Description of Artificial Sequence Synthetic Oligonucleotide
cctgctcgtcgctccgttggaa

SEQ ID NO: 211 - DNA, 22 bp, Artificial Sequence
  Description of Artificial Sequence Synthetic Oligonucleotide
gtgatcgccgtccctgtccgaa

SEQ ID NO: 212 - DNA, 22 bp, Artificial Sequence
  Description of Artificial Sequence Synthetic Oligonucleotide
ttccgaggcctgacccgaccaa

SEQ ID NO: 213 - DNA, 26 bp, Artificial Sequence
  Description of Artificial Sequence Synthetic Oligonucleotide
gtgtcgtggagtcggcaaaactatac

SEQ ID NO: 214 - DNA, 20 bp, Artificial Sequence
  Description of Artificial Sequence Synthetic Oligonucleotide
accgactccagctcccgaac

SEQ ID NO: 215 - DNA, 18 bp, Artificial Sequence
  Description of Artificial Sequence Synthetic Oligonucleotide
gtgtcgtggagtcggcaa

SEQ ID NO: 216 - DNA, 23 bp, Artificial Sequence
  Description of Artificial Sequence Synthetic Oligonucleotide
tgaagagatacgccctggttcct

SEQ ID NO: 217 - DNA, 25 bp, Artificial Sequence
  Description of Artificial Sequence Synthetic Oligonucleotide
gtgtcgtggagtcggcaaaggaacc

SEQ ID NO: 218 - protein, 10 aa, Artificial Sequence
  Description of Artificial Sequence Synthetic Peptide
EQKLISEEDL

SEQ ID NO: 219 - protein, 9 aa, Artificial Sequence
  Description of Artificial Sequence Synthetic Peptide
YPYDVPDYA

SEQ ID NO: 220 - protein, 11 aa, Artificial Sequence
  Description of Artificial Sequence Synthetic Peptide
YTDIEMNRLGK

SEQ ID NO: 221 - protein, 11 aa, Artificial Sequence
  Description of Artificial Sequence Synthetic Peptide
QPELAPEDPED

SEQ ID NO: 222 - protein, 14 aa, Artificial Sequence
  Description of Artificial Sequence Synthetic Peptide
GKPIPNPLLGLDST

SEQ ID NO: 223 - protein, 9 aa, Artificial Sequence
  Description of Artificial Sequence Synthetic Peptide
DYKDDDDKG

SEQ ID NO: 224 - DNA, 27 bp, Artificial Sequence
  Description of Artificial Sequence Synthetic Oligonucleotide
accgactccagctcccgaaacgaagag

SEQ ID NO: 225 - DNA, 24 bp, Artificial Sequence
  Description of Artificial Sequence Synthetic Oligonucleotide
aataccctgtagaaccgaatttgt